(19) United States
(12) Patent Application Publication
(10) Pub. No.: US 2026/0176288 A1
(43) Pub. Date: Jun. 25, 2026

(54) Anti-Inflammatory Compounds

(71) Applicant: Enanta Pharmaceuticals, Inc., Watertown, MA (US)

(72) Inventors: Sourav GHORAI, Waltham, MA (US); Robert LEON, Sharon, MA (US); Sean RAFFERTY, Watertown, MA (US); Jonathan THIELMAN, Cambridge, MA (US); William CASSELS, Watertown, MA (US); Samuel BARTLETT, Brighton, MA (US); Jiajun ZHANG, Cambridge, MA (US); Peilin XU, Watertown, MA (US); Yuk Ming SIU, Watertown, MA (US); Scott A. MITCHELL, Woburn, MA (US); Yat Sun OR, Waltham, MA (US)

(21) Appl. No.: 19/422,190

(22) Filed: Dec. 16, 2025

Related U.S. Application Data
(60) Provisional application No. 63/735,132, filed on Dec. 17, 2024.
(60) Provisional application No. 63/886,532, filed on Sep. 23, 2025.

(51) Int. Cl. C07D 498/04; A61K 31/404; A61K 31/4178; A61K 31/4184; A61K 31/4196; A61K 31/437; A61K 31/444; A61K 31/4545; A61K 31/4985; A61K 31/501; A61K 31/513; A61K 31/5383; C07D 403/10; C07D 405/14; C07D 471/04; C07D 487/04; C07D 519/00

(52) U.S. Cl. CPC C07D 498/04; A61K 31/404; A61K 31/4178; A61K 31/4184; A61K 31/4196; A61K 31/437; A61K 31/444; A61K 31/4545; A61K 31/4985; A61K 31/501; A61K 31/513; A61K 31/5383; C07D 403/10; C07D 405/14; C07D 471/04; C07D 487/04; C07D 519/00

(57) ABSTRACT

The invention provides compounds of Formula (I),

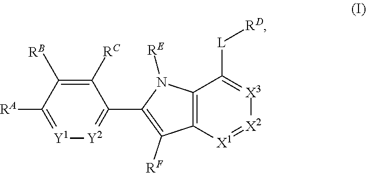

and pharmaceutically acceptable salts and pharmaceutical compositions thereof,
which are useful as inhibitors of STAT family members, and methods for using such compounds to treat, ameliorate or prevent an inflammatory disease or allergic disease, including asthma and atopic dermatitis associated with STAT proteins, especially associated with IL-4 signaling.

### RELATED APPLICATIONS

This application claims the benefit of U.S. Provisional Application No. 63/735,132, filed Dec. 17, 2024, and U.S. Provisional Application No. 63/886,532, filed Sep. 23, 2025. The entire teachings of the above applications are incorporated herein by reference.

### TECHNICAL FIELD

The present invention relates generally to compounds and pharmaceutical compositions useful as inhibitors of STAT family members. Specifically, the present invention relates to compounds that are useful in treating diseases mediated by STAT proteins.

### BACKGROUND OF THE INVENTION

Members of the Signal Transducer and Activator of Transcription (STAT) protein family are transcription factors that play central roles in cell processes including immunity, metabolism, proliferation, differentiation, apoptosis and angiogenesis. The human genome encodes seven STAT proteins: STAT1, STAT2, STAT3, STAT4, STAT5A, STAT5B, and STAT6. STAT3 and STAT6 have prominent roles in processes relevant to inflammatory and autoimmune diseases.

STAT3 is a pleotropic cytokine involved in host defense, development of hematopoietic stem cells (HSCs) and non-HSCs, metabolism, and autoimmunity. STAT3 plays a central role in the development of T helper 17 (Tₕ17) and T follicular helper (T_{fh}) cells and is a critical component of signaling pathways of inflammatory cytokines, including IL-6, IL-23, and IL-31. Therapeutic targeting of these cytokines with monoclonal antibodies has been used to treat autoimmune and autoinflammatory diseases. Inhibitors of JAK tyrosine kinases involved in promulgating inflammatory signaling upstream of STAT3 have been used as treatments for autoimmune disorders as well as in oncology for myeloproliferative neoplasms and solid tumors.

STAT6 is a necessary and critical node of the JAK/STAT signaling pathway that is activated upon interaction of the cytokines IL-4 and IL-13 with their receptor containing the IL-4Rα subunit. This process has been found to play a central role in the development of type 2 inflammatory diseases including asthma and atopic dermatitis. Antibodies targeting IL-4Rα can achieve therapeutic blockade of both IL-4 and IL-13 mediated signaling, while antibodies that selectively target the IL-13 cytokine have also been used in the clinic. Furthermore, the importance of this pathway is highlighted by missense variants of STAT6 that have recently been shown to protect against asthma and dampen the type 2 inflammatory response. WO2014182928, WO2023192960, WO2023164680, WO2023133336, WO2024071439, and WO2025236004, disclose small molecule STAT6 inhibitors, but an oral inhibitor of STAT6 has not been approved, and there remains a need in the art for novel therapeutic agents that treat or ameliorate inflammatory diseases associated with IL-4 and IL-13 signaling.

### SUMMARY OF THE INVENTION

The present invention relates to novel anti-inflammatory compounds, pharmaceutical compositions comprising such compounds, as well as methods to treat a subject in need of therapy with said compounds. Compounds of the present invention bind STAT family members, thereby inhibiting the transcription of genes mediated by STAT proteins, particularly STAT3 and STAT6.

The present invention provides compounds represented by Formula (I)

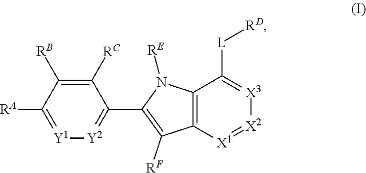

and pharmaceutically acceptable salts, N-oxides, esters and prodrugs thereof, wherein;
R^{A} is selected from the group consisting of:
1) Optionally substituted 3- to 12-membered heterocycloalkyl;
2) Optionally substituted aryl;
3) Optionally substituted arylalkyl;
4) Optionally substituted heteroaryl; and
5) Optionally substituted heteroarylalkyl;
R^{B} is selected from the group consisting of hydrogen, halogen, cyano, hydroxy, optionally substituted —C₁-C₈ alkyl, optionally substituted —C₂-C₈ alkenyl, optionally substituted —C₂-C₈ alkynyl, optionally substituted —C₁-C₈ alkoxy, optionally substituted —C₃-C₈ cycloalkyl, optionally substituted 3- to 8-membered heterocycloalkyl, optionally substituted aryl, optionally substituted heteroaryl, —C(O)N(R₁)(R₂), —NR₁R₂, —SR₁, —SO₂R₁, —N(R₁)C(O)(R₂), —N(R₁)C(O)O(R₂), and —N(R₁)S(O)₂(R₂);
R^{C} is hydrogen, optionally substituted —C₁-C₈ alkyl, or R^{C1};
R^{C1} is selected from the group consisting of halogen, cyano, hydroxy, optionally substituted —C₂-C₈ alkenyl, optionally substituted —C₂-C₈ alkynyl, optionally substituted —C₁-C₈ alkoxy, optionally substituted —C₃-C₈ cycloalkyl, optionally substituted 3- to 8-membered heterocycloalkyl, optionally substituted aryl, optionally substituted heteroaryl, —C(O)N(R₁)(R₂), —NR₁R₂, —SR₁, —SO₂R₁, —N(R₁)C(O)(R₂), —N(R₁)C(O)O(R₂), and —N(R₁)S(O)₂(R₂);
alternatively, R^{B} and R^{C} are taken together with the carbon atoms to which they are attached to form an optionally substituted —C₃-C₈ cycloalkyl or 3- to 8-membered heterocyclic ring system containing 1, 2, or 3 double bonds;
R^{D} is selected from the group consisting of hydrogen, optionally substituted —C₁-C₈ alkyl, optionally substituted —C₂-C₈ alkenyl, optionally substituted —C₃-C₁₂ cycloalkyl, optionally substituted 3- to 12-membered heterocycloalkyl, optionally substituted aryl, optionally substituted arylalkyl, optionally substituted heteroaryl, and optionally substituted heteroarylalkyl;
R^{E} is selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted arylalkyl, optionally substituted heteroarylalkyl, optionally substituted aryl, and optionally substituted heteroaryl;
R^{F} is hydrogen, halogen, or R^{F1};
R^{F1} is selected from the group consisting of:
1) Cyano;
2) Optionally substituted —C₁-C₈ alkyl;
3) Optionally substituted —C₃-C₈ cycloalkyl;
4) Optionally substituted 3- to 8-membered heterocycloalkyl;
5) Optionally substituted aryl;
6) Optionally substituted heteroaryl;
7) Optionally substituted —C₁-C₈ alkoxy; and
8) —C(O)N(R₁)(R₂);
Y¹ and Y² are independently selected from N or CR₄;
alternatively, both Y¹ and Y² are CR₄ and the two R₄ groups are taken together with the carbon atoms to which they are attached to form an optionally substituted —C₃-C₈ cycloalkyl or 3-to 8-membered heterocyclic ring system containing 1, 2, or 3 double bonds;
X¹, X², and X³ are each independently selected from N and CR₅;
L is selected from the group consisting of —C(R₆)₂N(R₇)—, —N(R₇)C(R₆)₂—, —C(R₆)₂O—, —OC(R₆)₂—, —SO₂N(R₇)—, —N(R₇)SO₂—, —C(O)N(R₇)—, —N(R₇)C(O)—, —C(O)₂—, —OC(O)—, —N(R₇)—, —S(O)₂—, —S(O)₂N(R₇)C(O)—, and —C(O)N(R₇)S(O)₂—;
Each R₁ and R₂ is independently selected from hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl, alternatively, R₁ and R₂ are taken together with the atom to which they are attached to form an optionally substituted 3-8 membered heterocyclic containing 0, 1, 2, or 3 double bonds;
Each R₄ is independently selected from the group consisting of hydrogen, halogen, cyano, hydroxy, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted —C₁-C₈ alkoxy, optionally substituted aryl, optionally substituted heteroaryl, —NR₁R₂, —SR₁, —SO₂R₁, —C(O)N(R₁)(R₂), —N(R₁)C(O)(R₂), —N(R₁)C(O)O(R₂), and —N(R₁)S(O)₂(R₂);
Each R₅ is independently selected from the group consisting of:
1) Hydrogen;
2) Halogen;
3) Cyano;
4) Hydroxy;
5) Optionally substituted alkyl;
6) Optionally substituted cycloalkyl;
7) Optionally substituted heterocycloalkyl;
8) Optionally substituted aryl;
9) Optionally substituted heteroaryl;
10) Optionally substituted —C₁-C₈ alkoxy;
11) —C(O)N(R₁)(R₂);
12) —N(R₁)C(O)(R₂);
13) —N(R₁)(R₂);
14) —SR₁;
15) —S(O)₂R₁;
16) —N(R₁)C(O)O(R₂);
17) —N(R₁)S(O)₂(R₂); and
18) —P(O)R₁R₂;
Each R₆ is independently hydrogen, halogen, optionally substituted —C₁-C₈ alkyl, optionally substituted —C₂-C₈ alkenyl, or optionally substituted —C₂-C₈ alkynyl; alternatively, two R₆ groups are taken together with the carbon atom to which they are attached to form an optionally substituted —C₃-C₈ cycloalkyl or 3- to 8 membered heterocycloalkyl ring system;
R₇ is selected from the group consisting of hydrogen, optionally substituted —C₁-C₈ alkyl, optionally substituted —C₃-C₈ cycloalkyl, optionally substituted 3- to 8 membered heterocycloalkyl, optionally substituted aryl and optionally substituted heteroaryl; and
provided that when R^{C} is hydrogen or optionally substituted —C₁-C₈ alkyl and R^{F} is hydrogen or halogen, then (1) L is not —C(O)₂—, —C(O)NH—, —CH₂O—, or —CH₂NH— and/or (2) R^{D} is not optionally substituted phenyl or optionally substituted 5- to 6-membered heteroaryl.

### DETAILED DESCRIPTION OF THE INVENTION

In one embodiment, the present invention provides a compound of Formula (I) as described above or a pharmaceutically acceptable salt thereof.

In certain embodiments of the compounds of Formula (I), R^{A} is optionally substituted aryl, optionally substituted heteroaryl, or optionally substituted 3- to 8-membered heterocycloalkyl.

In certain embodiments of the compounds of Formula (I), R^{A} is selected from one of the following by removing a hydrogen atom and is optionally substituted:

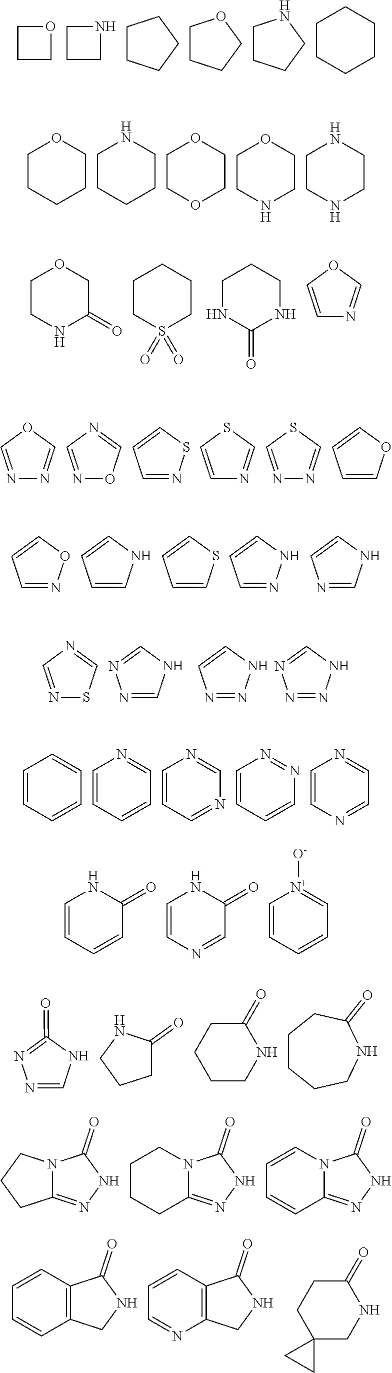

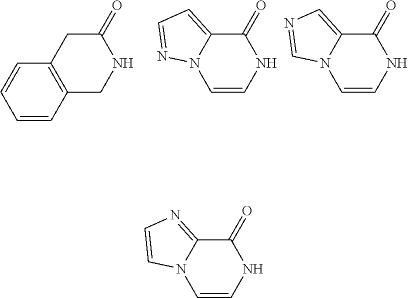

certain embodiments of the compounds of Formula (I), R^{A} is

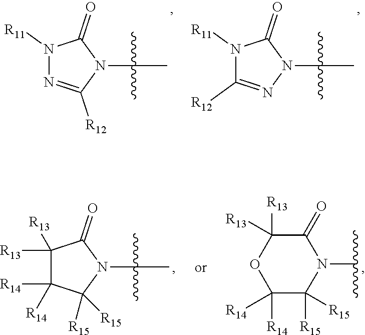

wherein
R₁₁ is selected from the group consisting of hydrogen, optionally substituted —C₁-C₈ alkyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl; R₁₂ is selected from the group consisting of hydrogen, halogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl; each R₁₃ is independently selected from the group consisting of hydrogen, halogen, optionally substituted —C₁-C₈ alkyl, and optionally substituted —C₁-C₈ alkoxy; alternatively, two R₁₃ groups are taken together with the carbon atom to which they are attached to form an optionally substituted —C₃-C₈ cycloalkyl or optionally substituted 3- to 8-membered heterocycloalkyl; each R₁₄ is independently selected from the group consisting of hydrogen, halogen, optionally substituted —C₁-C₈ alkyl, and optionally substituted —C₁-C₈ alkoxy; alternatively, two R₁₄ groups are taken together with the carbon atom to which they are attached to form an optionally substituted —C₃-C₈ cycloalkyl or optionally substituted 3- to 8-membered heterocycloalkyl; each R₁₅ is independently selected from the group consisting of hydrogen and optionally substituted —C₁-C₈ alkyl; alternatively, two R₁₅ groups are taken together with the carbon atom to which they are attached to form an optionally substituted —C₃-C₈ cycloalkyl or optionally substituted 3- to 8-membered heterocycloalkyl.
   alternatively, R^{A} is

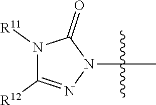

and R¹¹ and R¹² are taken together with the nitrogen atom and carbon atom to which they are respectively attached to form an optionally substituted heteroaryl or an optionally substituted 5-8 membered heterocycloalkyl;
alternatively, R^{A} is

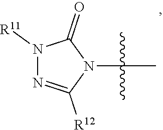

and R¹¹ and R¹² are taken together with the nitrogen atom and carbon atom to which they are respectively attached to form an optionally substituted 4-12 membered heterocycloalkyl which is bridged with the triazole ring;
alternatively, R^{A} is

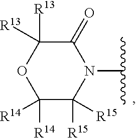

and one R¹³ and one R¹⁴ are taken together with the carbon atoms to which they are attached to form an optionally substituted —C₃-C₈ cycloalkyl or optionally substituted 4- to 8-membered heterocycloalkyl; and/or one R₁₄ and one R₁₅ are taken together with the carbon atoms to which they are attached to form an optionally substituted —C₄-C₈ cycloalkyl or optionally substituted 3- to 8-membered heterocycloalkyl;
alternatively, R^{A} is

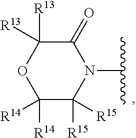

and one R¹³ and one R¹⁴ are taken together with the carbon atoms to which they are attached and the intervening oxygen atom to optionally substituted 4- to 12-membered heterocycloalkyl which is bridged with the morpholine ring;

In certain embodiments of the compounds of Formula (I), R^{A} is

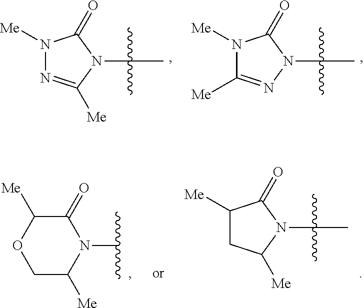 [SMILES: *n1c(C)nn(C)c1=O ; *n1nc(C)n(C)c1=O ; *N1C(=O)C(C)OCC1C ; *N1C(=O)C(C)CC1C]

In certain embodiments of the present invention, R^{A} is selected from one of the following

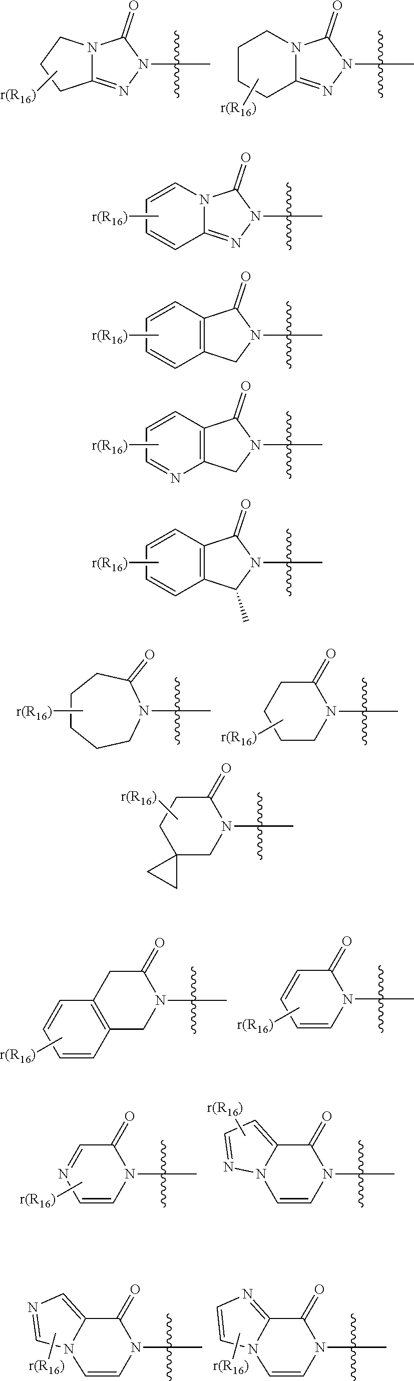

wherein each R₁₆ is independently selected from the group consisting of halogen, —CN, optionally substituted —C₁-C₈ alkyl, optionally substituted —C₃-C₈ cycloalkyl and optionally substituted —C₁-C₈ alkoxy; and r is 0, 1 or 2.

In certain embodiments of the compounds of Formula (I), R^{A} is

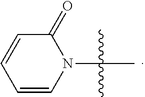 [SMILES: *n1ccccc1=O]

In certain embodiments of the compounds of Formula (I), R^{B} is hydrogen.

In certain embodiments of the compounds of Formula (I), R^{C} is hydrogen, halogen, optionally substituted —C₁-C₈ alkyl, optionally substituted —C₂-C₈ alkenyl, optionally substituted —C₂-C₈ alkynyl, or optionally substituted —C₁-C₈ alkoxy.

In certain embodiments of the compounds of Formula (I), R^{C} is hydrogen,

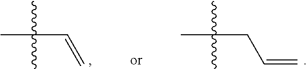

In certain embodiments of the compounds of Formula (I), Y¹ is CR₄, wherein R₄ is as previously defined.

In certain embodiments of the compounds of Formula (I), Y² is CR₄, wherein R₄ is as previously defined.

In certain embodiments of the compounds of Formula (I), Y¹ is CH, and Y² is CH.

In certain embodiments of the compounds of Formula (I), R^{E} is hydrogen or optionally substituted —C₁-C₈ alkyl.

In certain embodiments of the compounds of Formula (I), R^{E} is optionally substituted methyl, optionally substituted ethyl,

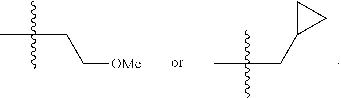 [SMILES: *CCOC ; "or" ; *CC1CC1 ; "."]

In certain embodiments of the compounds of Formula (I), R^{F} is hydrogen or optionally substituted —C₁-C₈ alkyl.

In certain embodiments of the compounds of Formula (I), R^{F} is optionally substituted methyl.

In certain embodiments of the compounds of Formula (I), X¹ is CR₅, wherein R₅ is as previously defined.

In certain embodiments of the compounds of Formula (I), X² is CR₅, wherein R₅ is as previously defined.

In certain embodiments of the compounds of Formula (I), X³ is CR₅, wherein R₅ is as previously defined.

In certain embodiments of the compounds of Formula (I), X¹ is CR₅, X² is CR₅, and X³ is CRS, wherein R₅ is as previously defined.

In certain embodiments of the compounds of Formula (I), X¹ is CH, X² is CR₅, and X³ is CH, and R₅ is as previously defined. Preferably R₅ is optionally substituted —C₁-C₈ alkyl, more preferably R₅ is —CF₃.

In certain embodiments of the compounds of Formula (I), L is —C(O)N(R₇)—, —C(O)N(R₇)SO₂—, or —SO₂N(R₇)—.

In certain embodiments of the compounds of Formula (I), L is —C(O)NH—, —C(O)NHSO₂—, or —SO₂NH—.

In certain embodiments of the compounds of Formula (I), L is —C(R₆)₂O—, or —C(R₆)₂N(R₇)—, wherein R₆ and R₇ are as previously defined.

In certain embodiments of the compounds of Formula (I), R^{D} is optionally substituted —C₃-C₁₂ cycloalkyl or optionally substituted 3- to 12-membered heterocycloalkyl.

In certain embodiments of the compounds of Formula (I), R^{D} is optionally substituted aryl or optionally substituted heteroaryl.

In certain embodiments of the compounds of Formula (I), R^{D} is optionally substituted arylalkyl or optionally substituted heteroarylalkyl.

In certain embodiments of the compounds of Formula (I), R^{D} is optionally substituted phenyl.

In certain embodiments of the compounds of Formula (I), R^{D} is optionally substituted benzyl.

In certain embodiments of the present invention, the compound of Formula (I) is represented by Formula (II),

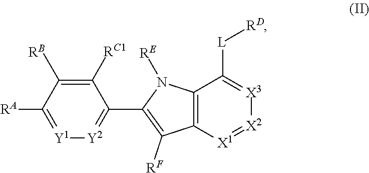

wherein R^{A}, R^{B}, R^{C1}, R^{D}, R^{E}, R^{F}, X¹, X², X³, Y¹, Y², and L are as previously defined.

In certain embodiments of the present invention, the compound of Formula (I) is represented by Formula (III),

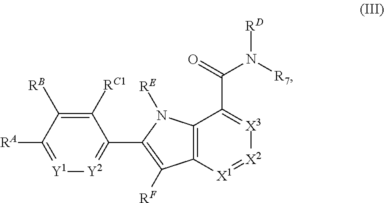

wherein R^{A}, R^{B}, R^{C1}, R^{D}, R^{E}, R^{F}, X¹, X², X³, Y¹, Y², and R₇ are as previously defined, preferably R^{C1} is optionally substituted —C₂-C₈ alkenyl or optionally substituted —C₂-C₈ alkynyl.

In certain embodiments of the present invention, the compound of Formula (I) is represented by Formula (IV),

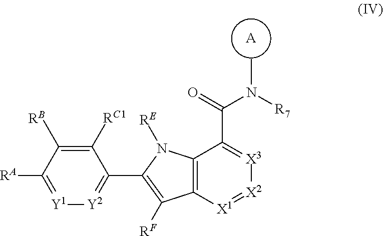

wherein \textcircled{A} is selected from the group consisting of optionally substituted —C₃-C₁₂ cycloalkyl, optionally substituted 3- to 12-membered heterocycloalkyl, optionally substituted aryl, and optionally substituted heteroaryl; R^{A}, R^{B}, R^{C1}, R^{E}, R^{F}, R₇, X¹, X², X³, Y¹, and Y² are as previously defined. Preferably R^{C1} is optionally substituted —C₂-C₈ alkenyl or optionally substituted —C₂-C₈ alkynyl.

In certain embodiments of the present invention, the compound of Formula (I) is represented by Formula (V),

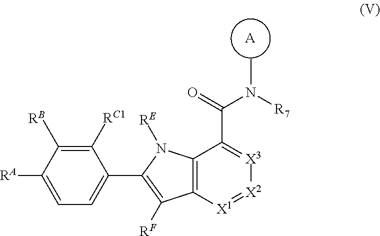

wherein \textcircled{A}, R^{A}, R^{B}, R^{C1}, R^{E}, R^{F}, R₇, X¹, X², and X³ are as previously defined. Preferably R^{C1} is optionally substituted —C₂-C₈ alkenyl or optionally substituted —C₂-C₈ alkynyl.

In certain embodiments of the present invention, the compound of Formula (I) is represented by Formula (VI),

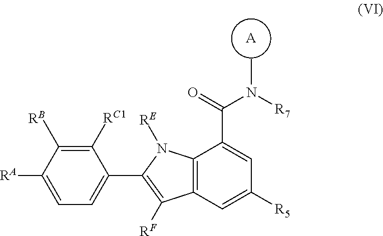

wherein \textcircled{A}, R^{A}, R^{B}, R^{C1}, R^{E}, R^{F}, R₅ and R₇ are as previously defined. Preferably R^{C1} is optionally substituted —C₂-C₈ alkenyl or optionally substituted —C₂-C₈ alkynyl.

In certain embodiments of the present invention, the compound of Formula (I) is represented by Formula (VII),

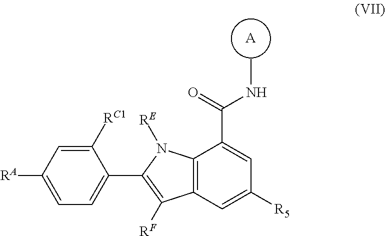

wherein \textcircled{A}, R^{A}, R^{C1}, R^{E}, R^{F}, and R₅ are as previously defined. Preferably R^{C1} is optionally substituted —C₂-C₈ alkenyl or optionally substituted —C₂-C₈ alkynyl.

In certain embodiments of the present invention, the compound of Formula (I) is represented by Formula (VIII),

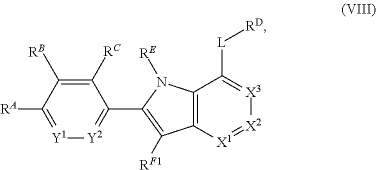

wherein R^{A}, R^{B}, R^{C}, R^{D}, R^{E}, R^{F1}, X¹, X², X³, Y¹, Y², and L are as previously defined.

In certain embodiments of the present invention, the compound of Formula (I) is represented by Formula (IX),

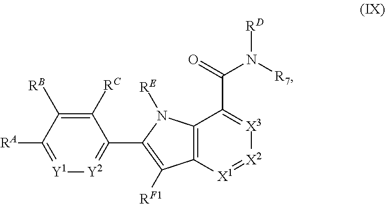

wherein R^{A}, R^{B}, R^{C}, R^{D}, R^{E}, R^{F1}, X¹, X², X³, Y¹, Y², and R₇ are as previously defined. Preferably R^{F1} is selected from the group consisting of optionally substituted —C₁-C₈ alkyl, optionally substituted —C₁-C₈ cycloalkyl, and optionally substituted 3- to 8-membered heterocycloalkyl.

In certain embodiments of the present invention, the compound of Formula (I) is represented by Formula (X),

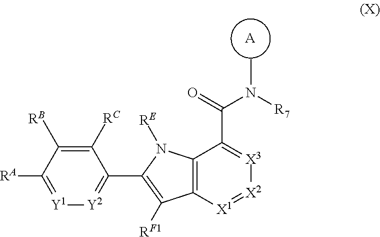

wherein \textcircled{A}, R^{A}, R^{B}, R^{C}, R^{E}, R^{F1}, R₇, X¹, X², X³, Y¹, and Y² are as previously defined. Preferably R^{F1} is selected from the group consisting of optionally substituted —C₁-C₈ alkyl, optionally substituted —C₁-C₈ cycloalkyl, and optionally substituted 3- to 8-membered heterocycloalkyl.

In certain embodiments of the present invention, the compound of Formula (I) is represented by Formula (XI),

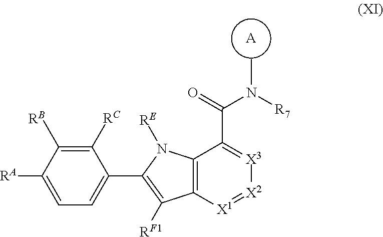

wherein \textcircled{A}, R^{A}, R^{B}, R^{C}, R^{E}, R^{F1}, R₇, X¹, X², and X³ are as previously defined. Preferably R^{F1} is selected from the group consisting of optionally substituted —C₁-C₈ alkyl, optionally substituted —C₁-C₈ cycloalkyl, and optionally substituted 3- to 8-membered heterocycloalkyl.

In certain embodiments of the present invention, the compound of Formula (I) is represented by Formula (XII),

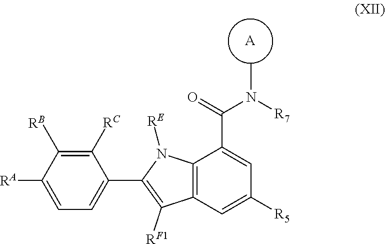

wherein \textcircled{A}, R^{A}, R^{B}, R^{C}, R^{E}, R^{F1}, R₅ and R₇ are as previously defined. Preferably R^{F1} is selected from the group consisting of optionally substituted —C₁-C₈ alkyl, optionally substituted —C₁-C₈ cycloalkyl, and optionally substituted 3- to 8-membered heterocycloalkyl.

In certain embodiments of the present invention, the compound of Formula (I) is represented by Formula (XIII),

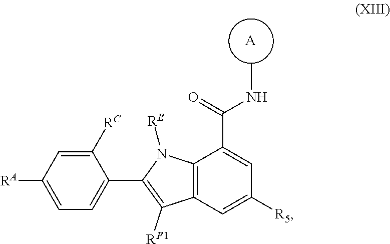

wherein \textcircled{A}, R^{A}, R^{C}, R^{E}, R^{F1}, and R₅ are as previously defined. Preferably R^{F1} is selected from the group consisting of optionally substituted —C₁-C₈ alkyl, optionally substituted —C₁-C₈ cycloalkyl, and optionally substituted 3- to 8-membered heterocycloalkyl.

In certain embodiments of the present invention, the compound of Formula (I) is represented by one of Formulae (XIV-1)˜(XIV-3),

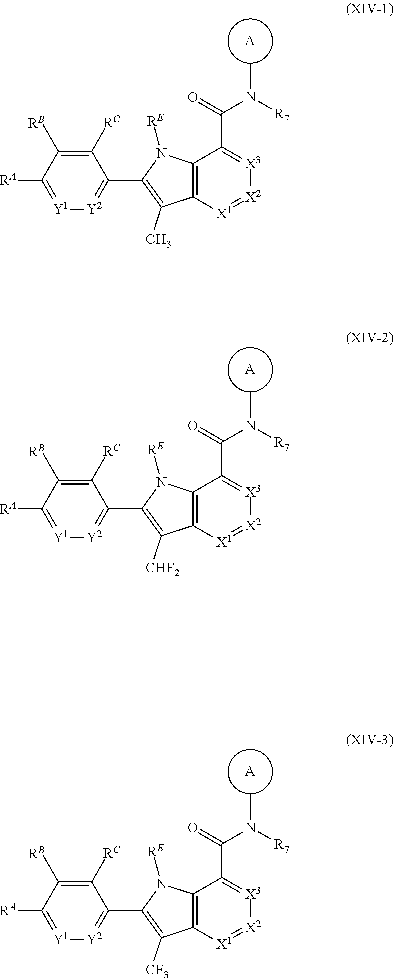

wherein \textcircled{A}, R^{A}, R^{B}, R^{C}, R^{E}, R₇, X¹, X², X³, Y¹, and Y² are as previously defined.

In certain embodiments of the present invention, the compound of Formula (I) is represented by one of Formulae (XV-1)˜(XV-3),

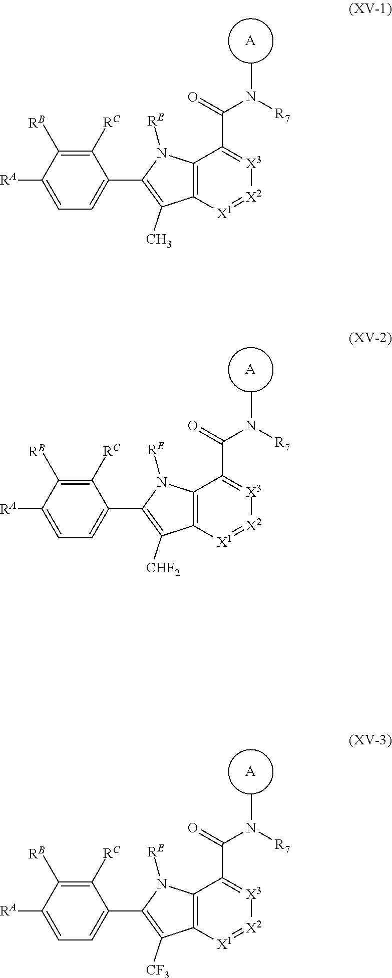

wherein \textcircled{A}, R^{A}, R^{B}, R^{C}, R^{E}, R₇, X¹, X², X³, and Y² are as previously defined.

In certain embodiments of the present invention, the compound of Formula (I) is represented by one of Formulae (XVI-1)˜(XVI-3),

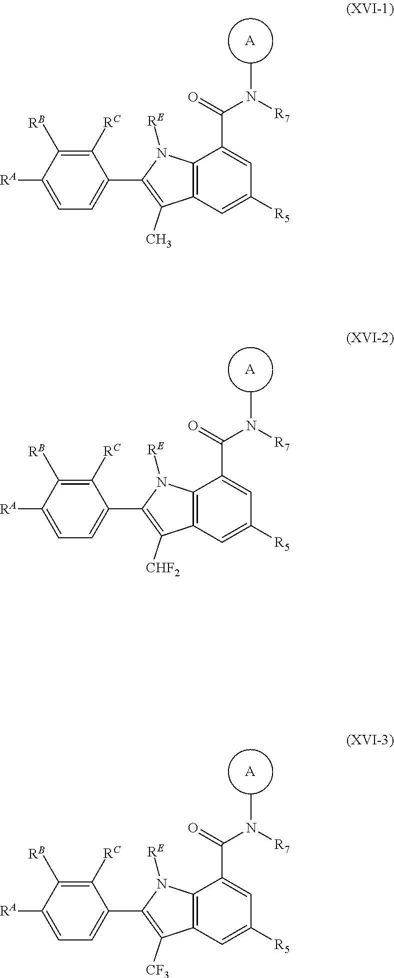

wherein \textcircled{A}, R^{A}, R^{B}, R^{C}, R^{E}, R₅ and R₇ are as previously defined.

In certain embodiments of the present invention, the compound of Formula (I) is represented by one of Formulae (XVII-1)˜(XVII-3),

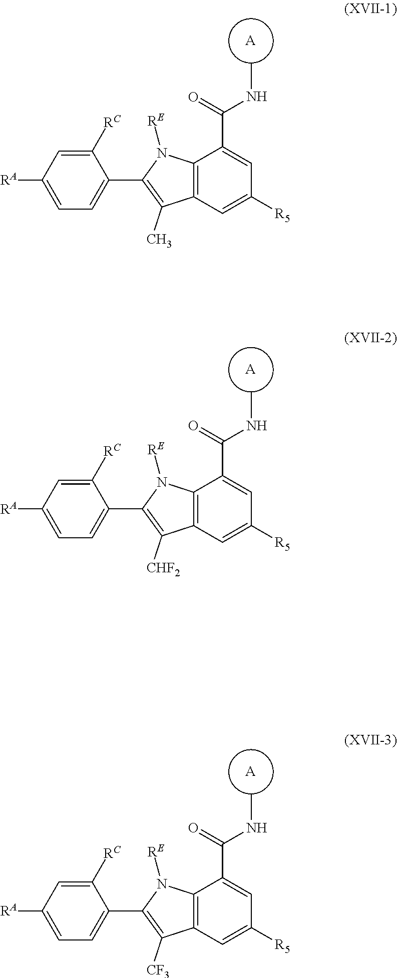

wherein \textcircled{A}, R^{A}, R^{C}, R^{E}, and R₅ are as previously defined.

In certain embodiments of the present invention, the compound of Formula (I) is represented by one of Formulae (XIX-1)˜(XIX-3),

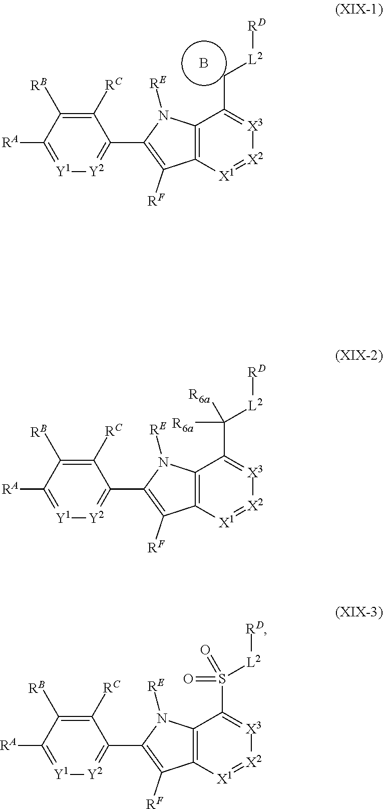

wherein each R₆ₐ is independently selected from the group consisting of optionally substituted alkyl and halogen; \textcircled{B} is an optionally substituted C₃-C₈ cycloalkyl or optionally substituted 3- to 8-membered heterocycloalkyl; L² is selected from the group consisting of —N(R⁷)— and —O—; and R^{A}, R^{B}, R^{C}, R^{D}, R^{E}, R^{F}, X¹, X², X³, Y¹, Y², and R⁷ are as previously defined.

In certain embodiments of the present invention, the compound of Formula (I) is represented by one of Formulae (XX-1)˜(XX-6),

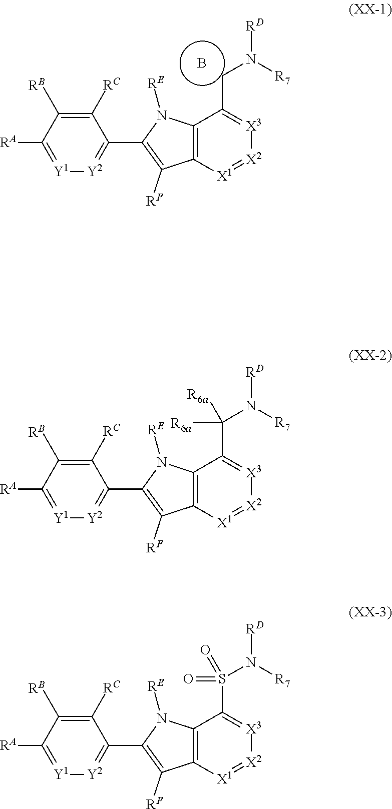

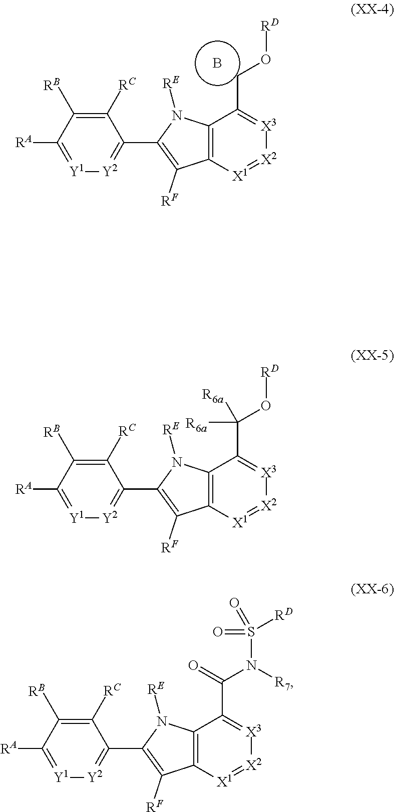

wherein \textcircled{B}, R₆ₐ, R₇, R^{A}, R^{B}, R^{C}, R^{D}, R^{E}, R^{F}, X¹, X², X³, Y¹, and Y² are as previously defined.

In certain embodiments of the present invention, the compound of Formula (I) is represented by one of Formulae (XXI-1)˜(XXI-6),

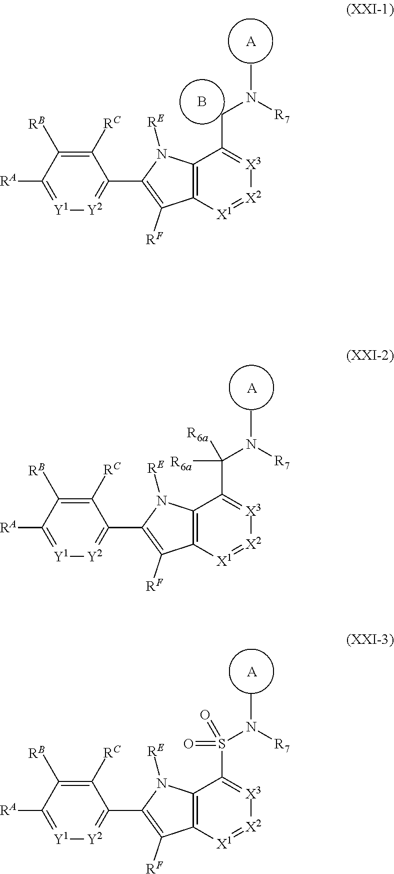

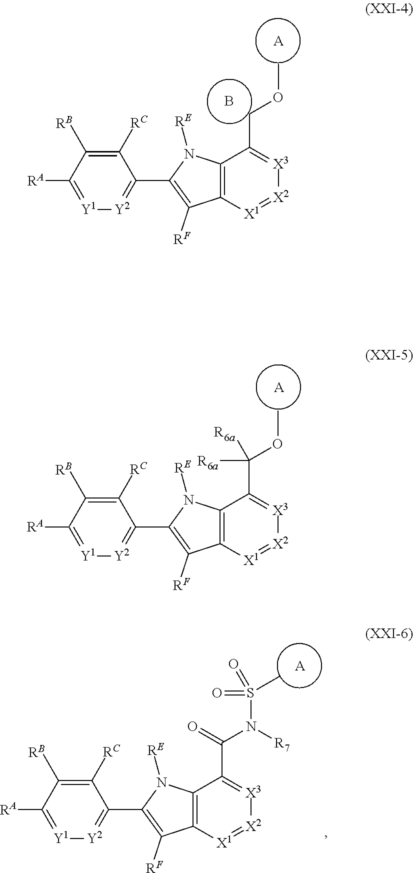

wherein \textcircled{A}, \textcircled{B}, R₆ₐ, R₇, R^{A}, R^{B}, R^{C}, R^{E}, R^{F}, X¹, X², X³, Y¹, and Y² are as previously defined.

In certain embodiments of the present invention, the compound of Formula (I) is represented by Formulae (XXII-1)˜(XXII-6),

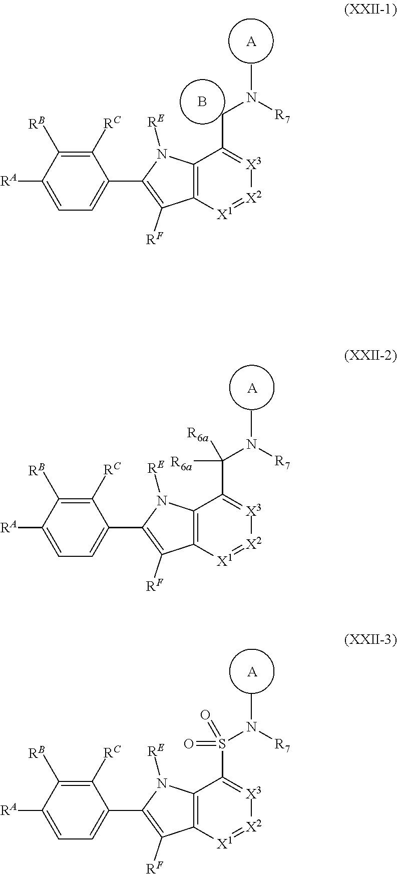

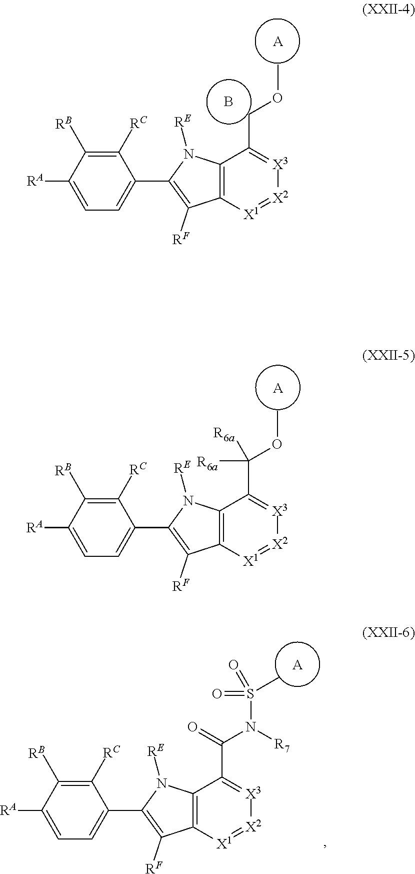

wherein \textcircled{A}, \textcircled{B}, R₆ₐ, R₇, R^{A}, R^{B}, R^{C}, R^{E}, R^{F}, X¹, X², and X³ are as previously defined.

In certain embodiments of the present invention, the compound of Formula (I) is represented by Formulae (XXIII-1)˜(XXIII-6),

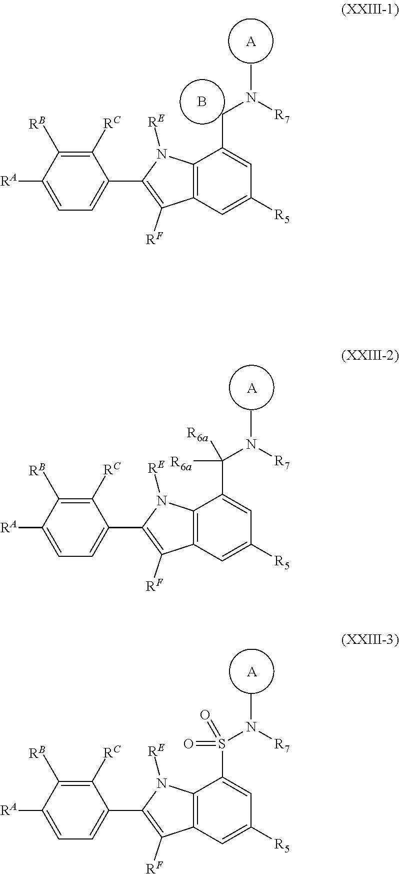

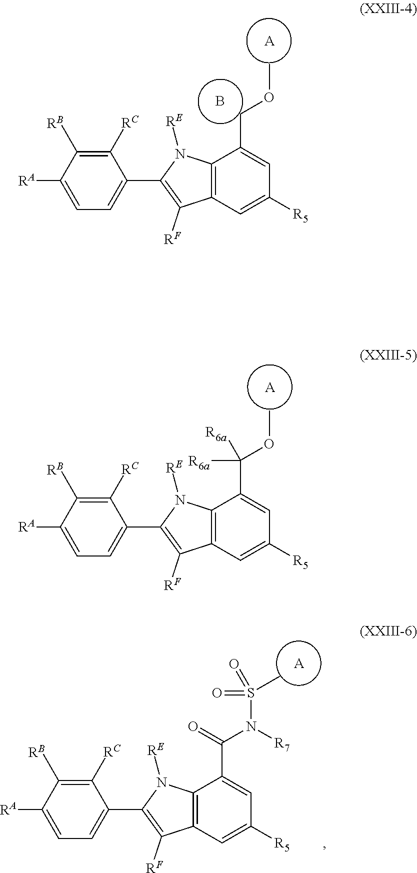

wherein \textcircled{A}, \textcircled{B}, R₅, R₆ₐ, R₇, R^{A}, R^{B}, R^{C}, R^{E}, and R^{F} are as previously defined.

In certain embodiments of the present invention, the compound of Formula (I) is represented by Formulae (XXIV-1)˜(XXIV-6),

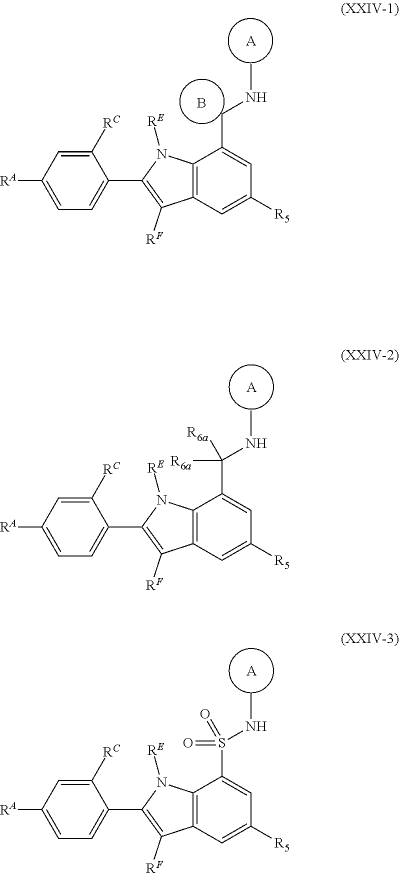

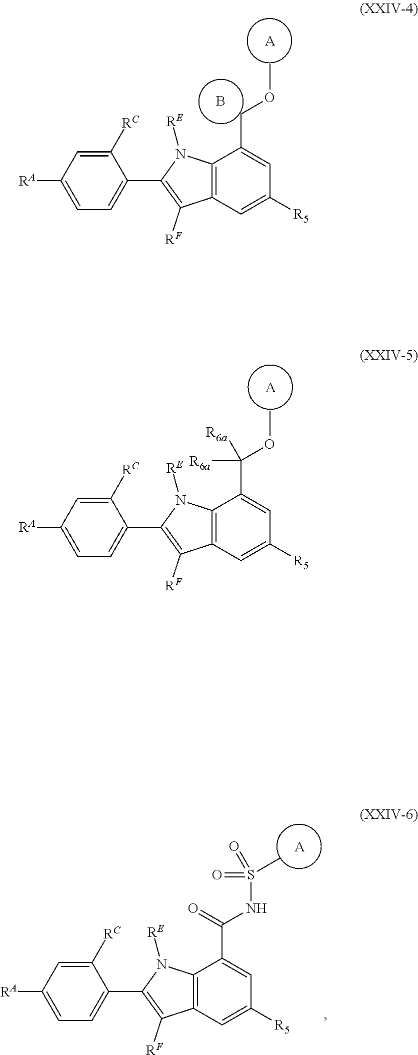

wherein \textcircled{A}, \textcircled{B}, R₅, R₆ₐ, R^{A}, R^{C}, R^{E}, and R^{F} are as previously defined. Preferably, \textcircled{B} is

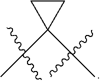 [SMILES: *C1(*)CC1]

In certain embodiments of the present invention, the compound of Formula (I) is represented by one of Formulae (XXV-1)˜(XXV-5),

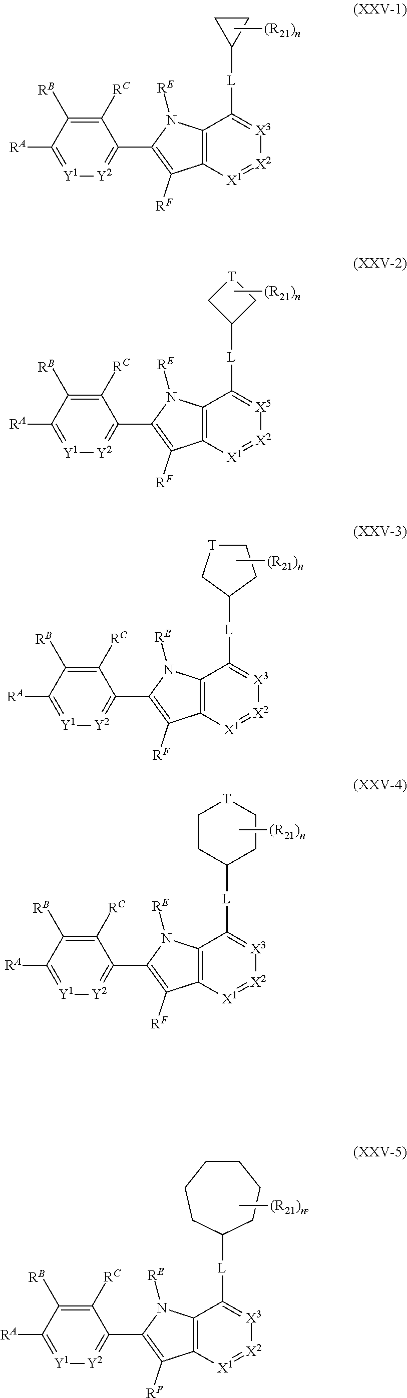

wherein;
   each R₂₁ is independently selected from the group consisting of:
   1) Hydrogen;
   2) Cyano;
   3) Halogen;
   4) Hydroxy;
   5) Optionally substituted C₁-C₈ alkyl;
   6) Optionally substituted aryl;
   7) Optionally substituted heteroaryl;
   8) Optionally substituted —C₁-C₈ alkoxy;
   9) —C(O)N(R₁)(R₂); and
   10) —CO₂H; and
   n is 0, 1 or 2;
   alternatively, n is 2 and two geminal R₂₁ groups are taken together with the carbon atom to which they are attached to form a spiro carbocyclic or heterocyclic ring spired, or n is 2 and two adjacent R₂₁ groups are taken together with the carbon atoms to which they are attached to form a fused carbocyclic or heterocyclic ring; or n is 2 and two remote R₂₁ groups are taken together with the carbon atoms to which they are connected to form a bridge; T is selected from the group consisting of O, S, SO₂, NR₂₂, and C(R₂₃)₂. R₂₂ is selected from the group consisting of:
   1) Hydrogen;
   2) Optional substituted —C₁-C₈ alkyl;
   3) Optionally substituted —C₃-C₈ cycloalkyl;
   4) Optionally substituted 3- to 8-membered heterocycloalkyl;
   5) Optionally substituted aryl;
   6) Optionally substituted heteroaryl;
   7) —C(O)R₁;
   8) —C(O)OR₁;
   9) —C(O)N(R₁)(R₂); and
   10) —S(O)₂R₁; and
   each R₂₃ is independently selected from the group consisting of:
   1) Hydrogen;
   2) Halogen;
   3) Optional substituted —C₁-C₈ alkyl;
   4) Optionally substituted —C₃-C₈ cycloalkyl;
   5) Optionally substituted 3- to 8-membered heterocycloalkyl;
   6) Optionally substituted aryl;
   7) Optionally substituted heteroaryl;
   8) —C(O)R₁;
   9) —C(O)OR₁;
   10) —C(O)N(R₁)(R₂); and
   11) —S(O)₂R₁;
and R^{A}, R^{B}, R^{C}, R^{E}, R^{F}, X¹, X², X³, L, Y¹ and Y² are as previously defined.

In certain embodiments of the present invention, the compound of Formula (I) is represented by one of Formulae (XXVI-1)˜(XXVI-5),

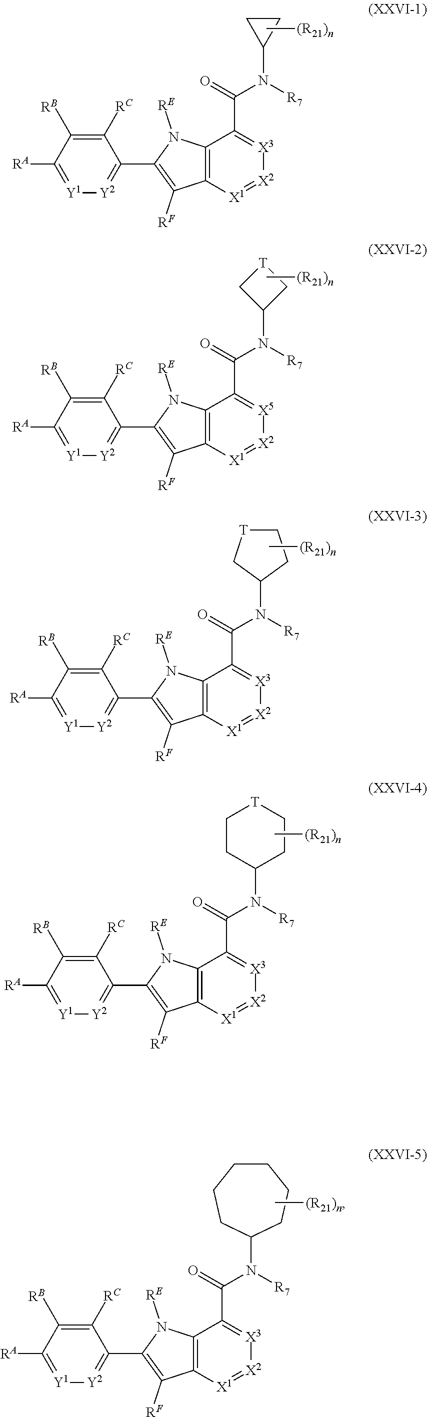

wherein R^{A}, R^{B}, R^{C}, R^{E}, R^{F}, X¹, X², X³, Y¹, Y², R₇, R₂₁, n, and T are as previously defined.

In certain embodiments of the present invention, the compound of Formula (I) is represented by one of Formulae (XXVI-1a)˜(XXVI-5a),

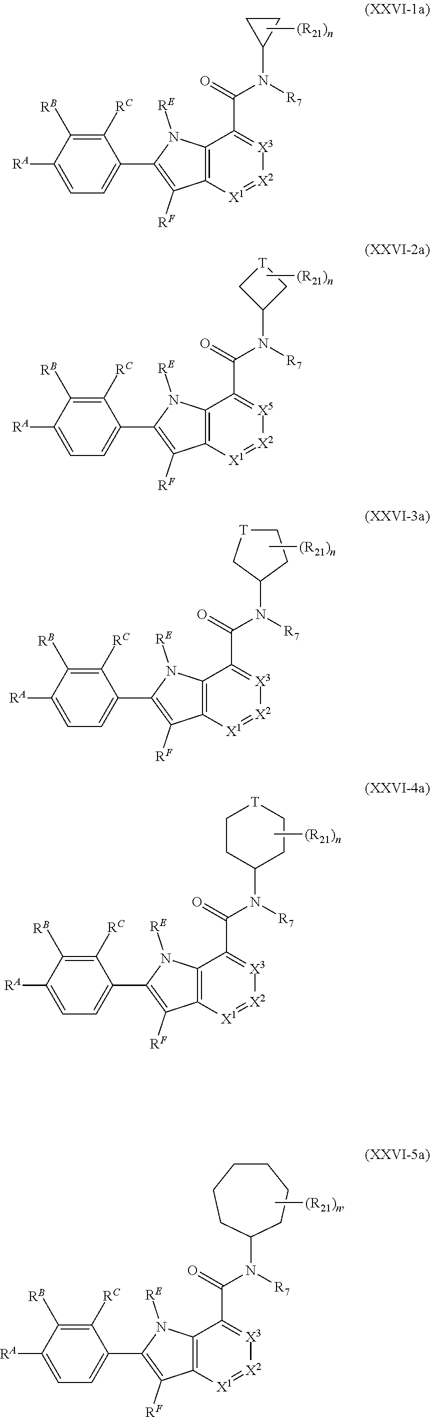

wherein R^{A}, R^{B}, R^{C}, R^{E}, R^{F}, X¹, X², X³, R₇, R₂₁, n, and T are as previously defined.

In certain embodiments of the present invention, the compound of Formula (I) is represented by one of Formulae (XXVII-1)˜(XXVII-5),

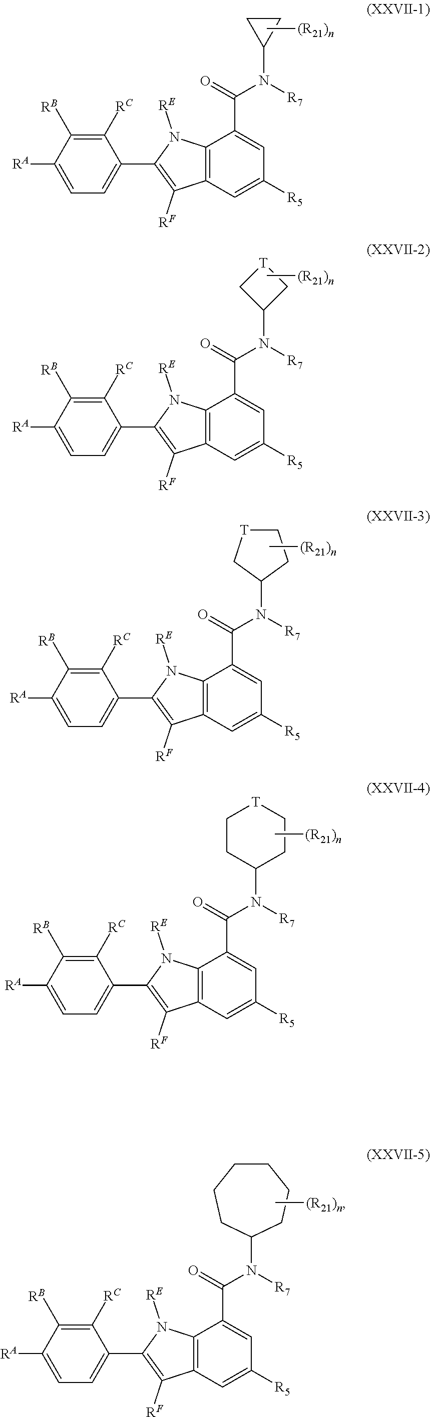

wherein R^{A}, R^{B}, R^{C}, R^{E}, R^{F}, R₅, R₇, R₂₁, n, and T are as previously defined.

In certain embodiments of the present invention, the compound of Formula (I) is represented by one of Formulae (XXVIII-1)˜(XXVIII-5),

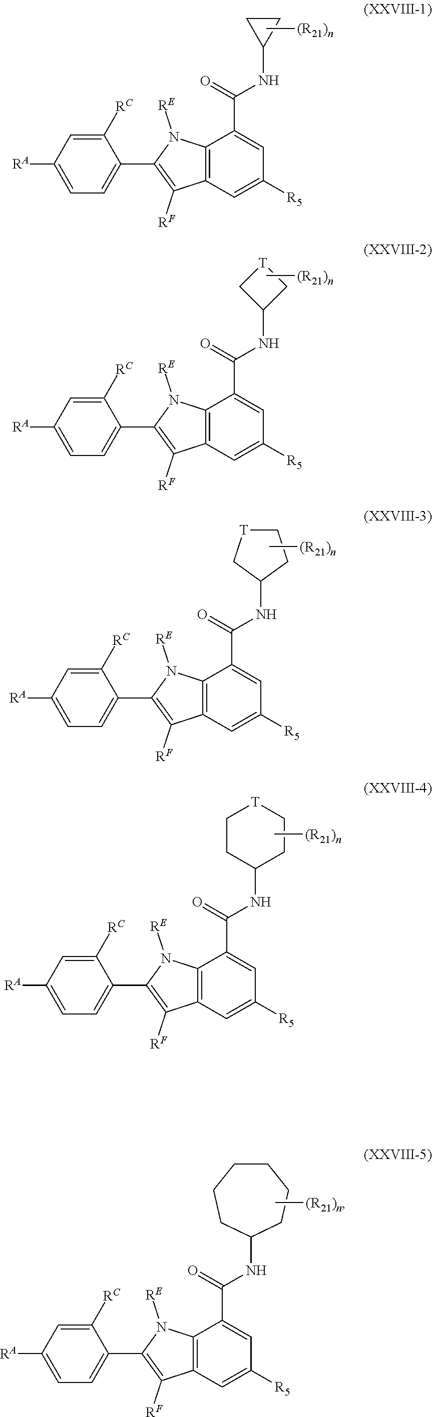

wherein R^{A}, R^{C}, R^{E}, R^{F}, R₅, R₂₁, n, and T are as previously defined.

In certain embodiments of the present invention, the compound of Formula (I) is represented by Formula (XXIX),

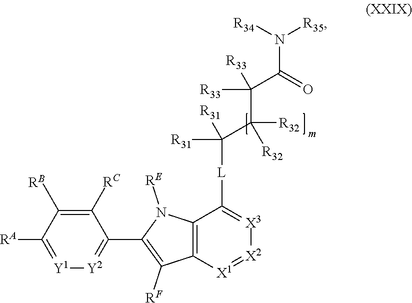

wherein each R₃₁ is selected from the group consisting of hydrogen, halogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted heteroaryl, and —C(O)N(R₁)(R₂);
   each R₃₂ is selected from the group consisting of hydrogen, halogen, and optionally substituted —C₁-C₈ alkyl;
   alternatively, two R₃₂ groups are taken together with the carbon atom to which they are attached to form an optionally substituted —C₃-C₈cycloalkyl or optionally substituted 3- to 8-membered heterocycloalkyl;
   each R₃₃ is selected from the group consisting of hydrogen, halogen, and —C₁-C₈ optionally substituted alkyl;
   R₃₄ is R₁; R₃₅ is R₂; m is 0, 1, or 2; and R₁, R₂, R^{A}, R^{B}, R^{C}, R^{E}, R^{F}, L, X¹, X², X³, Y¹, and Y² are as previously defined.

In certain embodiments of the compounds of Formula (XXIX), each R₃₁, R₃₂, and R₃₃ is hydrogen, halogen or C₁-C₄-alkyl. In certain embodiments, each R₃₁, R₃₂, and R₃₃ is hydrogen, fluoro, methyl or ethyl. In certain embodiments, each R₃₁, R₃₂, and R₃₃ is hydrogen.

In certain embodiments of the compounds of Formula (XXIX), R₃₄ and R₃₅ are independently hydrogen or C₁-C₄-alkyl. In certain embodiments R₃₄ and R₃₅ are independently hydrogen, methyl or ethyl. In certain embodiments R₃₄ and R₃₅ are both hydrogen.

In certain embodiments of the present invention, the compound of Formula (I) is represented by Formula (XXX),

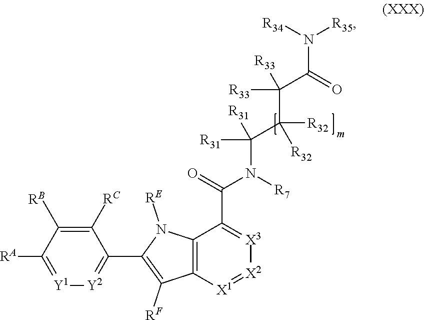

wherein R₇, R₃₁, R₃₂, R₃₃, R₃₄, R₃₅, m, R^{A}, R^{B}, R^{C}, R^{E}, R^{F}, X¹, X², X³, Y¹, and Y² are as previously defined.

In certain embodiments of the present invention, the compound of Formula (I) is represented by Formula (XXXI),

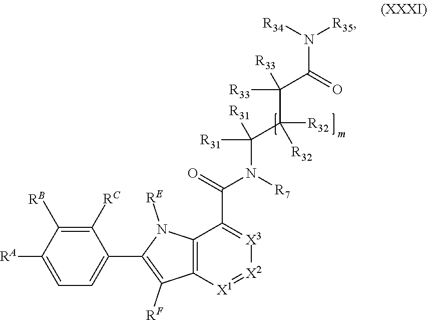

wherein R₇, R₃₁, R₃₂, R₃₃, R₃₄, R₃₅, m, R^{A}, R^{B}, R^{C}, R^{E}, R^{F}, X¹, X², and X³ are as previously defined.

In certain embodiments of the present invention, the compound of Formula (I) is represented by Formula (XXXII),

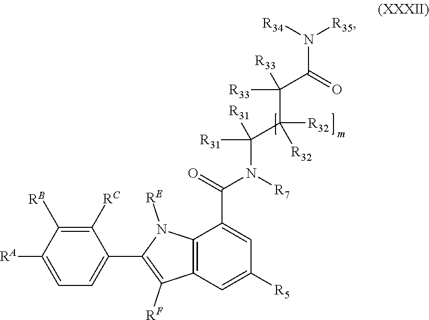

wherein R₅, R₇, R₃₁, R₃₂, R₃₃, R₃₄, R₃₅, m, R^{A}, R^{B}, R^{C}, R^{E}, and R^{F} are as previously defined.

In certain embodiments of the present invention, the compound of Formula (I) is represented by Formula (XXXIII),

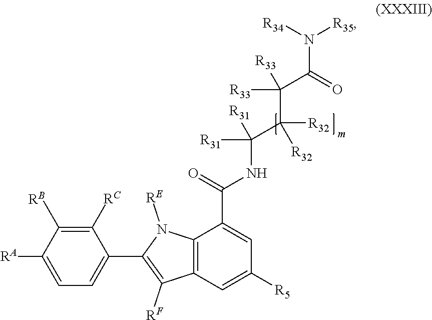

wherein R₅, R₃₁, R₃₂, R₃₃, R₃₄, R₃₅, m, R^{A}, R^{C}, R^{E}, and R^{F} are as previously defined.

In certain embodiments of the present invention, the compound of Formula (I) is represented by one of Formulae (XXXIV-1)˜(XXXIV-5),

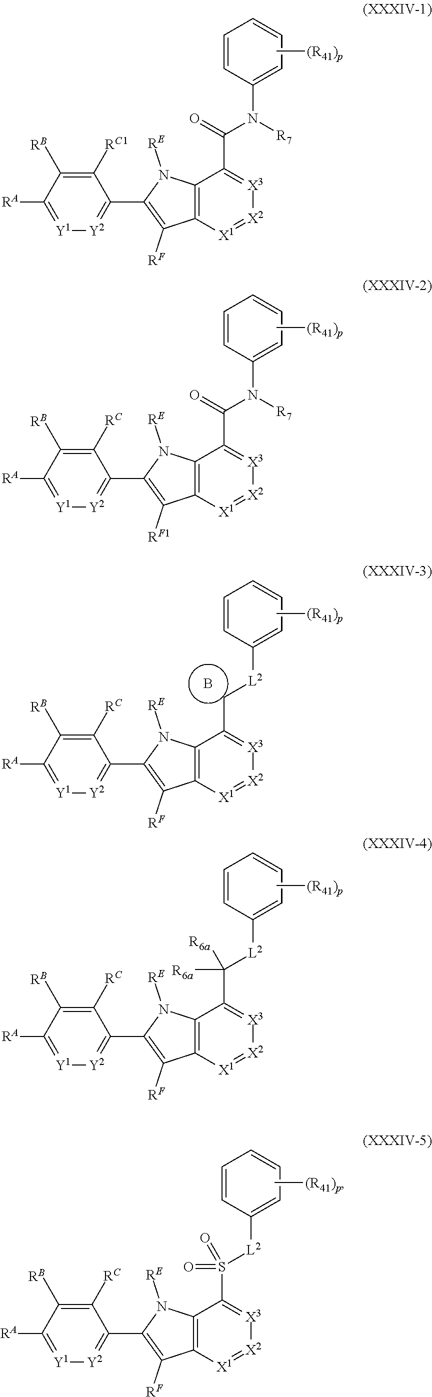

wherein each R₄₁ is independently selected from the group consisting of halogen, cyano, hydroxy, optionally substituted —C₁-C₈ alkyl, optionally substituted —C₂-C₈ alkenyl, optionally substituted —C₂-C₈ alkynyl, optionally substituted —C₁-C₈ alkoxy, optionally substituted —C₃-C₈ cycloalkyl, optionally substituted 3- to 8-membered heterocycloalkyl, optionally substituted aryl, optionally substituted heteroaryl, optionally substituted arylalkyl, optionally substituted heteroarylalkyl, —C(O)N(R₁)(R₂), —NR₁R₂, —SR₁, —SO₂R₁, —N(R₁)C(O)(R₂), —N(R₁)C(O)O(R₂), —N(R₁)S(O)₂(R₂), and —NR₁ₐC(O)NR₁R₂; R₁ₐ is R₁; p is 0, 1, 2, 3, or 4, preferably, p is 1 or 2; and R₁, R₂, R₇, \textcircled{B}, R^{A}, R^{B}, R^{C}, R^{C1}, R^{E}, R^{F}, R^{F1}, X¹, X², X³, Y¹, Y², and L² are as previously defined.

In certain embodiments of the present invention, the compound of Formula (I) is represented by one of Formulae (XXXIV-1a)˜(XXXIV-5a),

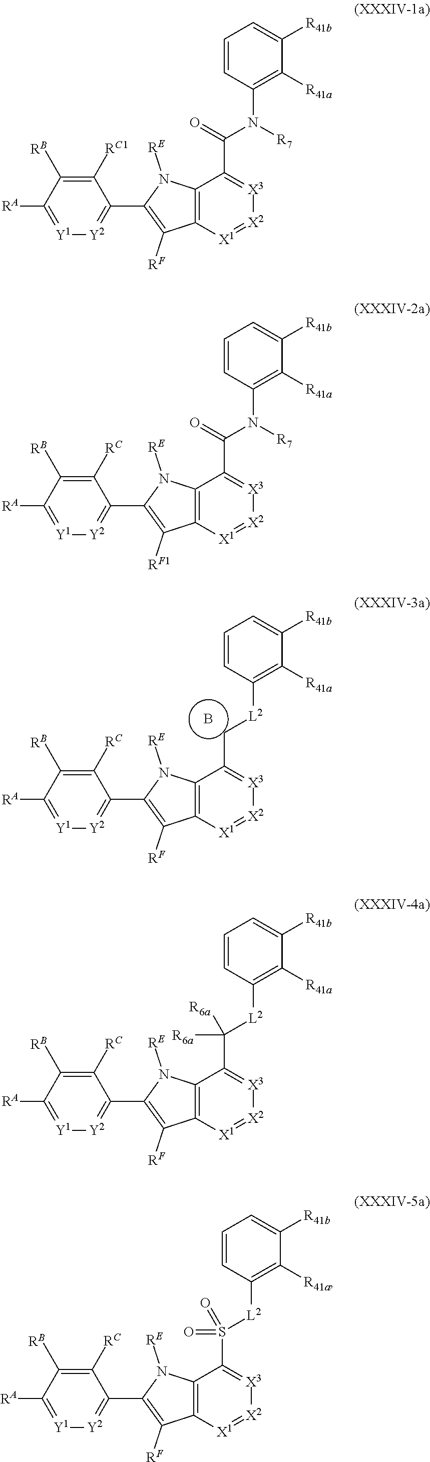

wherein R₄₁ₐ is R₄₁; R_{41b} is R₄₁, and R₄₁ₐ and R_{41b} are the same or different; R₇, R₄₁, \textcircled{B}, R^{A}, R^{B}, R^{C}, R^{C1}, R^{E}, R^{F}, R^{F1}, X¹, X², X³, Y¹, Y², and L² are as previously defined.

In certain embodiments of the present invention, the compound of Formula (I) is represented by one of Formulae (XXXIV-1b)˜(XXXIV-5b),

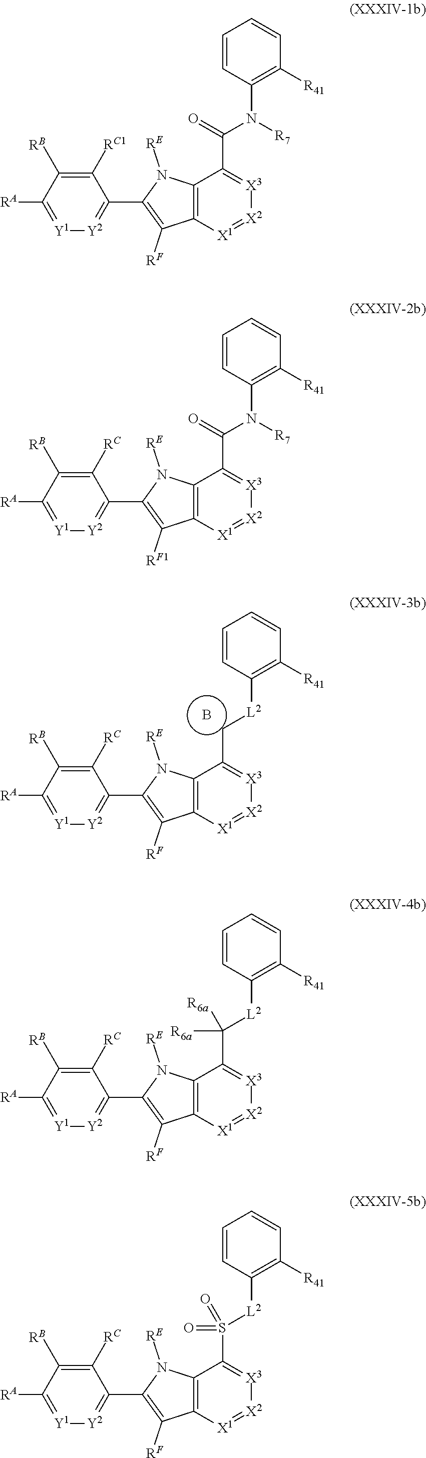

wherein R₄₁, R₇, \textcircled{B}, R^{A}, R^{B}, R^{C}, R^{C1}, R^{E}, R^{F}, R^{F1}, X¹, X², X³, Y¹, Y², and L² are as previously defined.

In certain embodiments of the present invention, the compound of Formula (I) is represented by one of Formulae (XXXV-1)˜(XXXV-6),

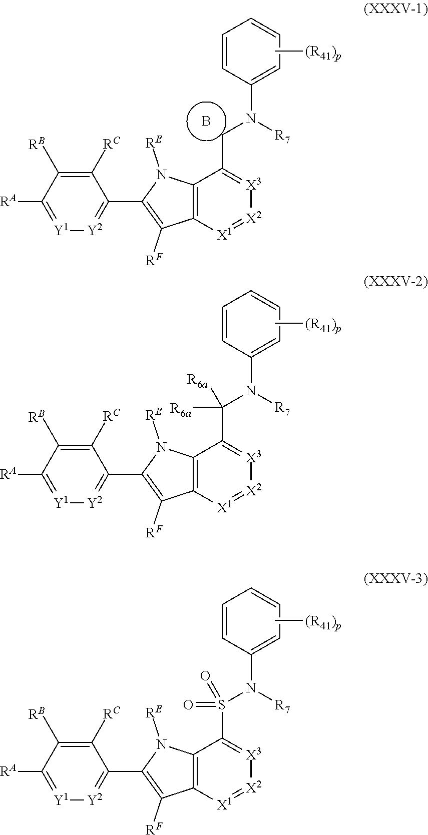

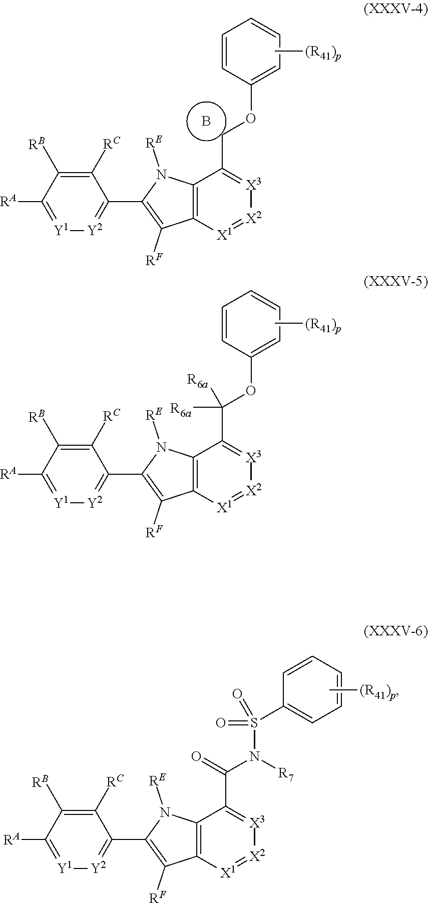

wherein R₄₁, p, \textcircled{B}, R^{A}, R^{B}, R^{C}, R^{C1}, R^{E}, R^{F}, R^{F1}, X, X², X³, y, Y², R₆ₐ, and R₇ are as previously defined. Preferably p is 1 or 2.

In certain embodiments of the present invention, the compound of Formula (I) is represented by one of Formulae (XXXV-1a)˜(XXXV-6a),

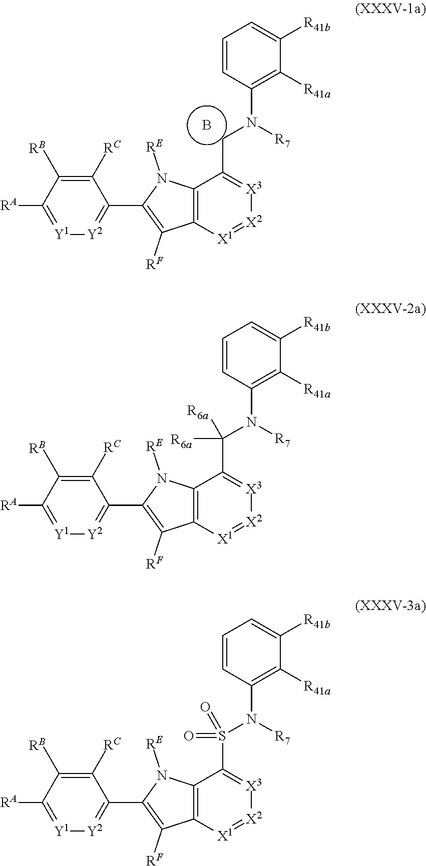

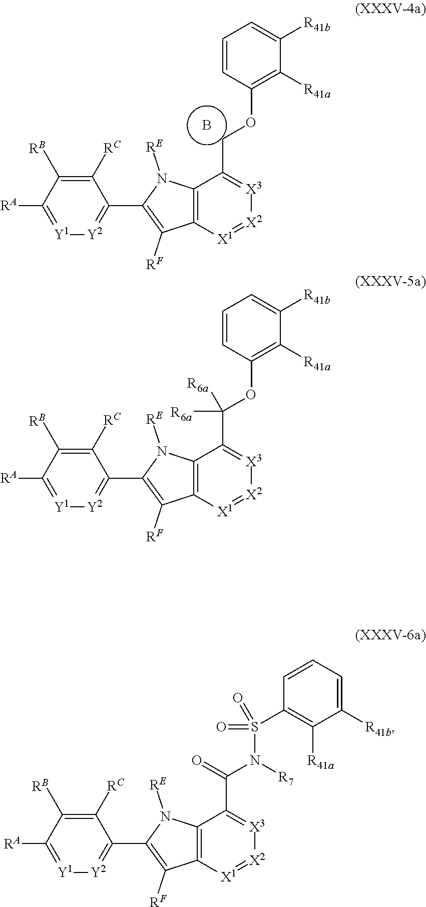

wherein R₄₁ₐ, R_{41b}, \textcircled{B}, R^{A}, R^{B}, R^{C}, R^{E}, R^{F}, X¹, X², X³, Y¹, Y², R₆ₐ, and R₇ are as previously defined.

In certain embodiments of the present invention, the compound of Formula (I) is represented by one of Formulae (XXXV-1b)˜(XXXV-6b),

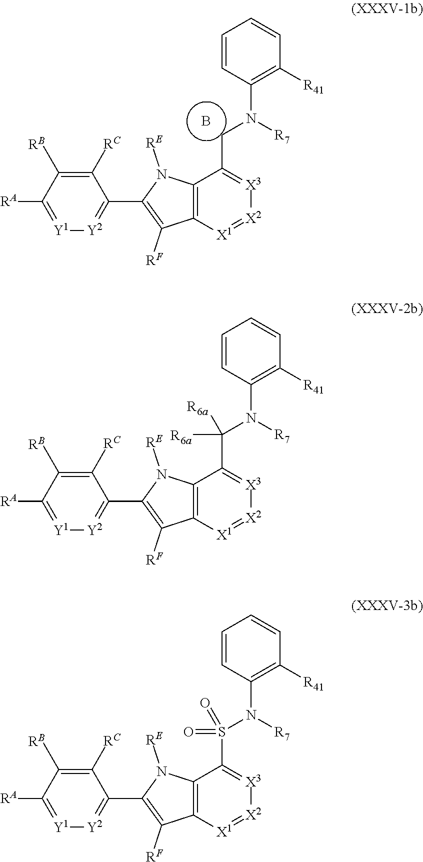

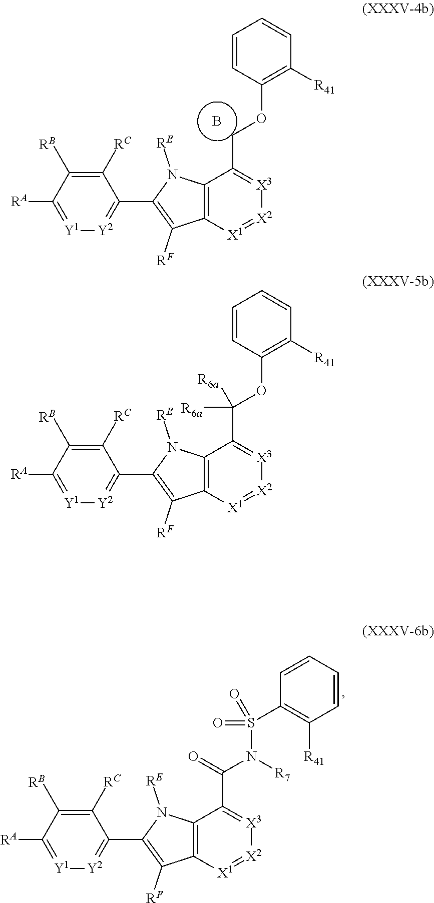

wherein R₄₁, \textcircled{B}, R^{A}, R^{B}, R^{C}, R^{E}, R^{F}, X¹, X², X³, Y¹, Y², R₆ₐ, and R₇ are as previously defined.

In certain embodiments of the present invention, the compound of Formula (I) is represented by one of Formulae (XXXVI-1)˜(XXXVI-5),

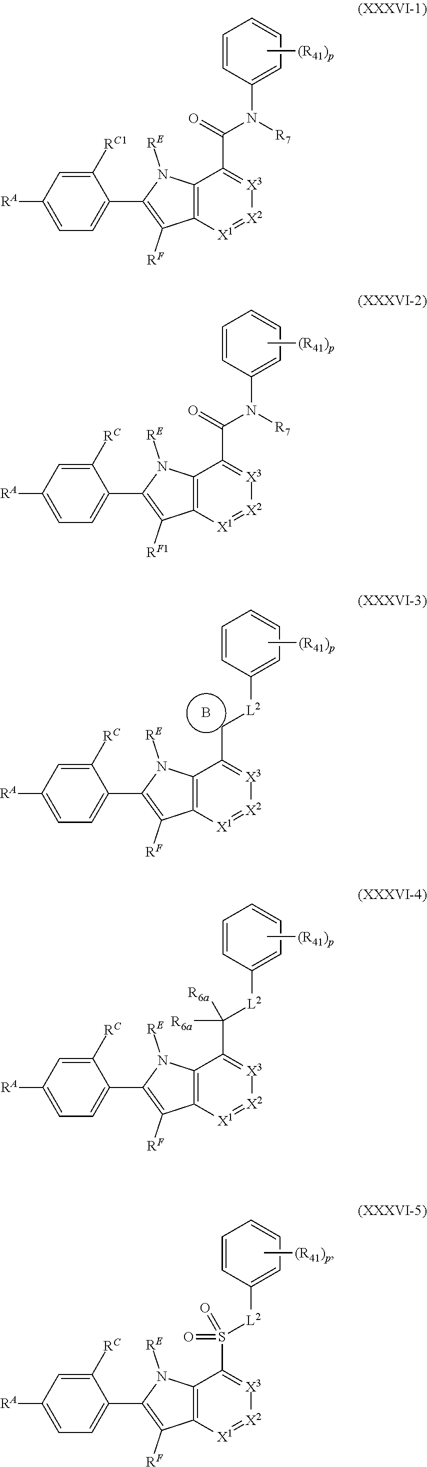

wherein R₄₁, p, \textcircled{B}, R^{A}, R^{C}, R^{C1}, R^{E}, R^{F}, R^{F1}, X¹, X², X³, L², R₆ₐ, and R₇ are as previously defined.

In certain embodiments of the present invention, the compound of Formula (I) is represented by one of Formulae (XXXVI-1a)˜(XXXVI-5a),

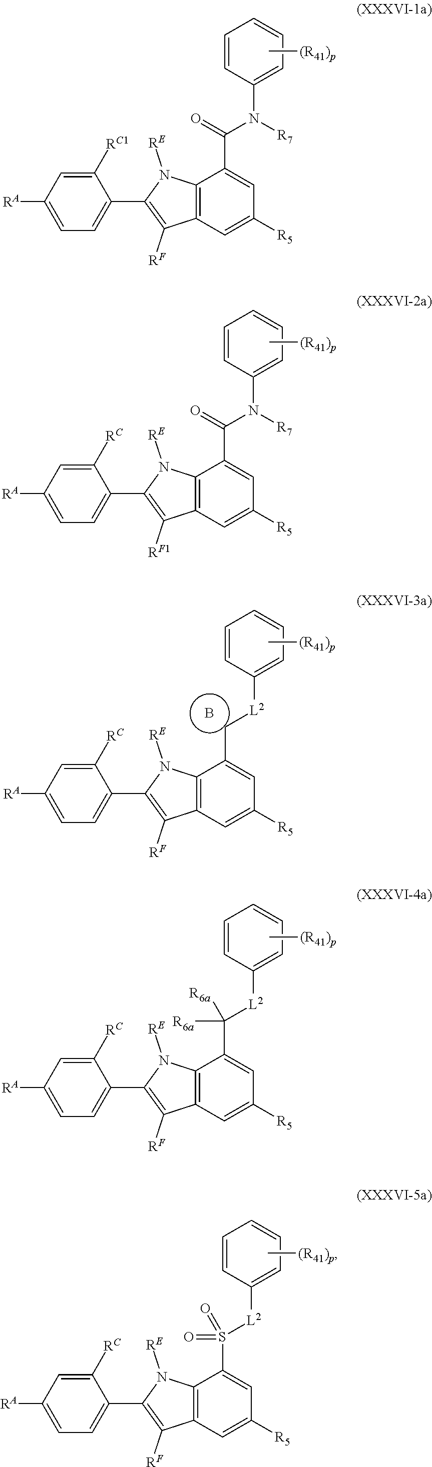

wherein R₄₁, p, \textcircled{B}, R^{A}, R^{C}, R^{C1}, R^{E}, R^{F}, R^{F1} L², R₅, R₆ₐ, and R₇ are as previously defined.

In certain embodiments of the present invention, the compound of Formula (I) is represented by one of Formulae (XXXVI-1b)˜(XXXVI-5b),

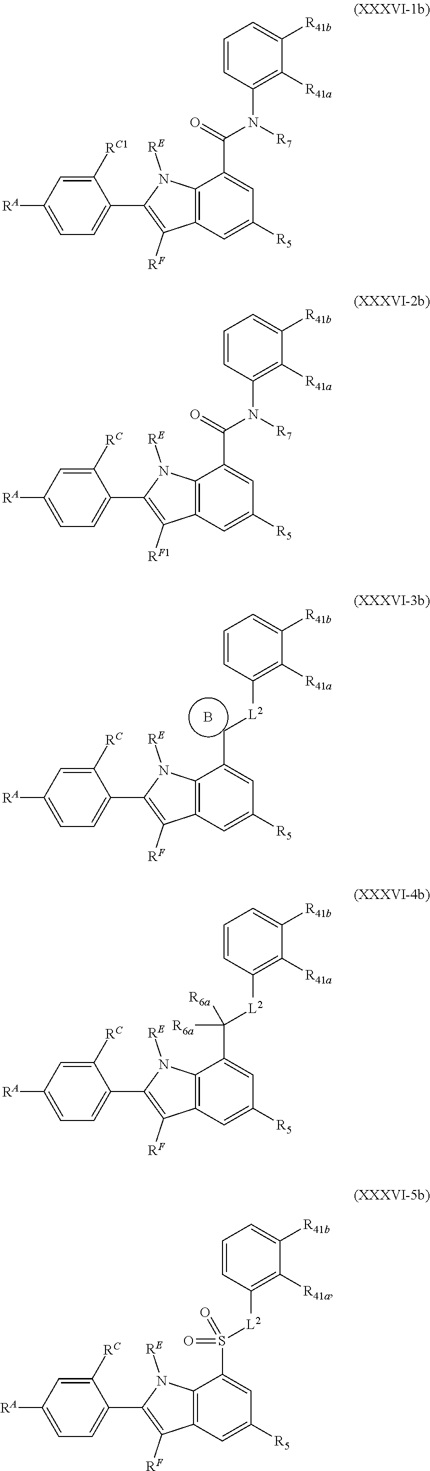

wherein R₄₁ₐ, R_{41b}, \textcircled{B}, R^{A}, R^{C}, R^{C1}, R^{E}, R^{F}, R^{F1}, L², R₅, R₆ₐ, and R₇ are as previously defined.

In certain embodiments of the present invention, the compound of Formula (I) is represented by one of Formulae (XXXVI-1c)˜(XXXVI-5c),

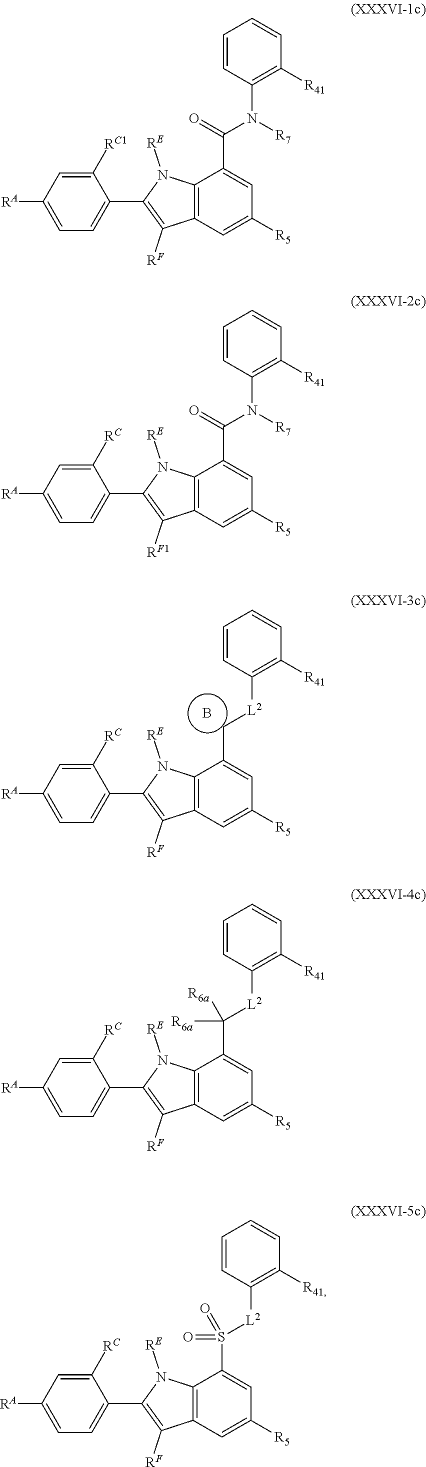

wherein R₄₁, \textcircled{B}, R^{A}, R^{C}, R^{C1}, R^{E}, R^{F}, R^{F1} L², R₅, R₆ₐ, and R₇ are as previously defined.

In certain embodiments of the present invention, the compound of Formula (I) is represented by one of Formulae (XXXVII-1)˜(XXXVII-6),

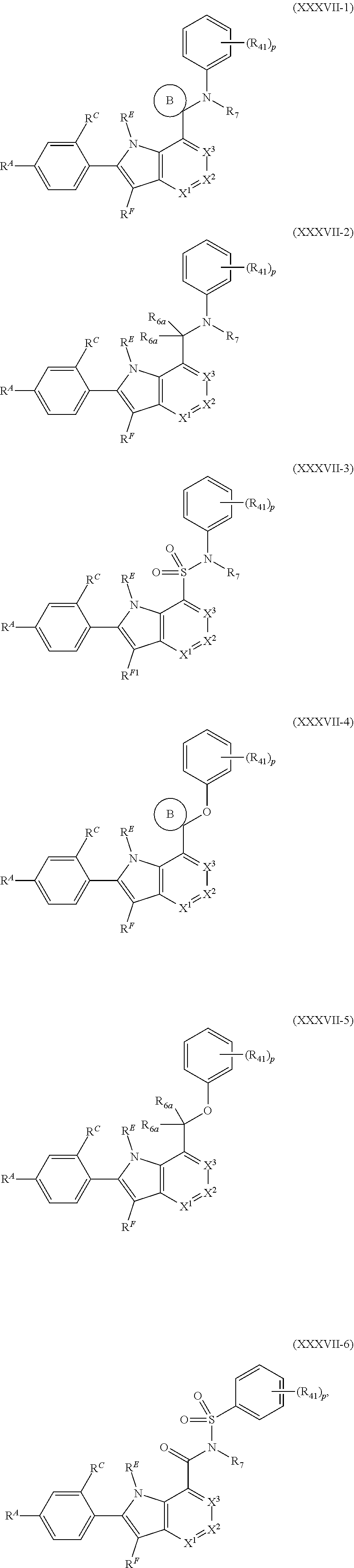

wherein R₄₁, p, \textcircled{B}, R^{A}, R^{C}, R^{E}, R^{F}, X¹, X², X³, R₆ₐ, and R₇ are as previously defined. Preferably p is 1 or 2.

In certain embodiments of the present invention, the compound of Formula (I) is represented by one of Formulae (XXXVII-1a)˜(XXXVII-6a),

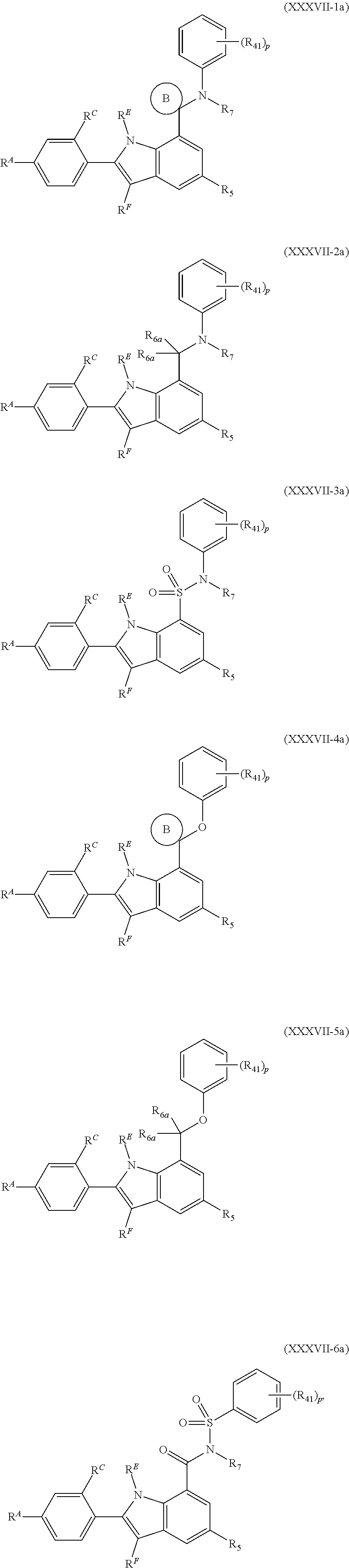

wherein R₄₁, p, \textcircled{B}, R^{A}, R^{C}, R^{E}, R^{F}, R₅, R₆ₐ, and R₇ are as previously defined. Preferably p is 1 or 2.

In certain embodiments of the present invention, the compound of Formula (I) is represented by one of Formulae (XXXVII-1b)˜(XXXVII-6b),

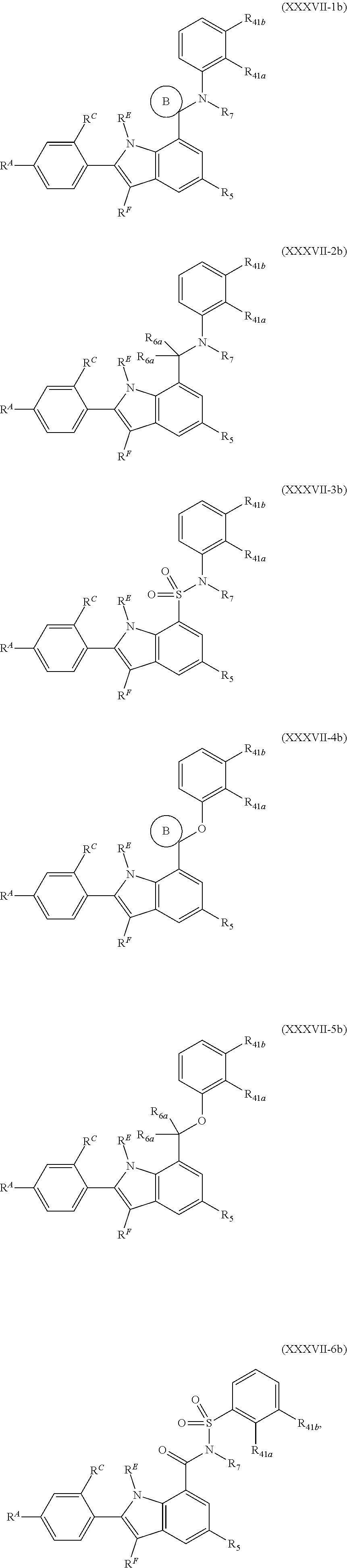

wherein R₄₁ₐ, R_{41b}, \textcircled{B}, R^{A}, R^{C}, R^{E}, R^{F}, R₅, R₆ₐ, and R₇ are as previously defined.

In certain embodiments of the present invention, the compound of Formula (I) is represented by one of Formulae (XXXVII-1c)˜(XXXVII-6c),

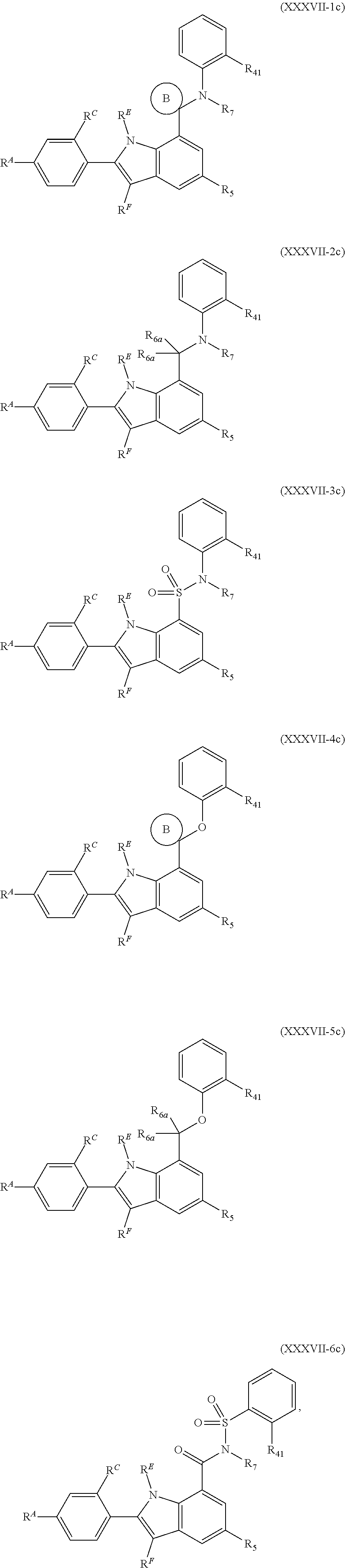

wherein R₄₁, \textcircled{B}, R^{A}, R^{C}, R^{E}, R^{F}, R₅, R₆ₐ, and R₇ are as previously defined.

In certain embodiments of the present invention, the compound of Formula (I) is represented by one of Formulae (XXXVI-1a)˜(XXXVI-5a) and (XXXVII-1a)˜(XXXVII-6a), wherein R^{A} is

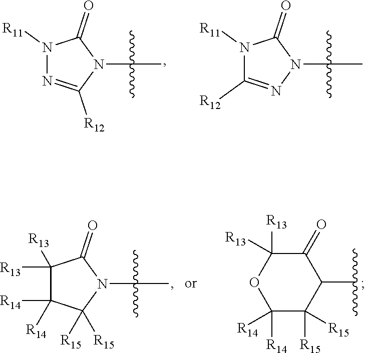

and R₁₁, R₁₂, R₁₃, R₁₄, R₁₅ are as previously defined.

In certain embodiments of the present invention, the compound of Formula (I) is represented by one of Formulae (XXXVI-1a)˜(XXXVI-5a) or (XXXVII-1a)˜(XXXVII-6a), wherein R^{A} is

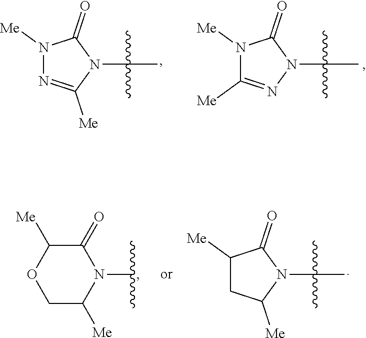

In certain embodiments of the present invention, the compound of Formula (I) is represented by Formula (XXXVIII),

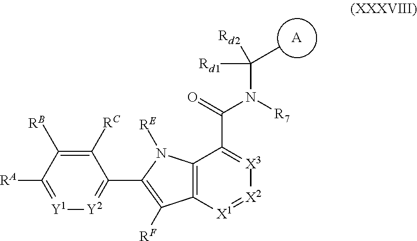

wherein R_{d1} and R_{d2} are each independently selected from hydrogen, halogen, and optionally substituted —C₁-C₆ alkyl; and \textcircled{A}, R^{A}, R^{B}, R^{C}, R^{E}, R^{F}, R₇, X¹, X², X³, Y¹, and Y² are as previously defined.

In certain embodiments of the present invention, the compound of Formula (I) is represented by Formula (XXXIX),

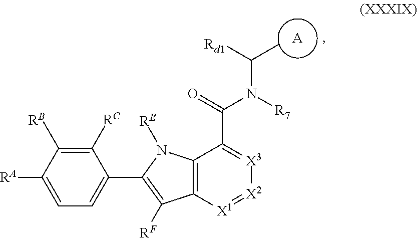

wherein R_{d1}, \textcircled{A}, R^{A}, R^{B}, R^{C}, R^{E}, R^{F}, R₇, X¹, X², and X³ are as previously defined.

In certain embodiments of the present invention, the compound of Formula (I) is represented by one of Formulae (XXXX-1)˜(XXXX-4),

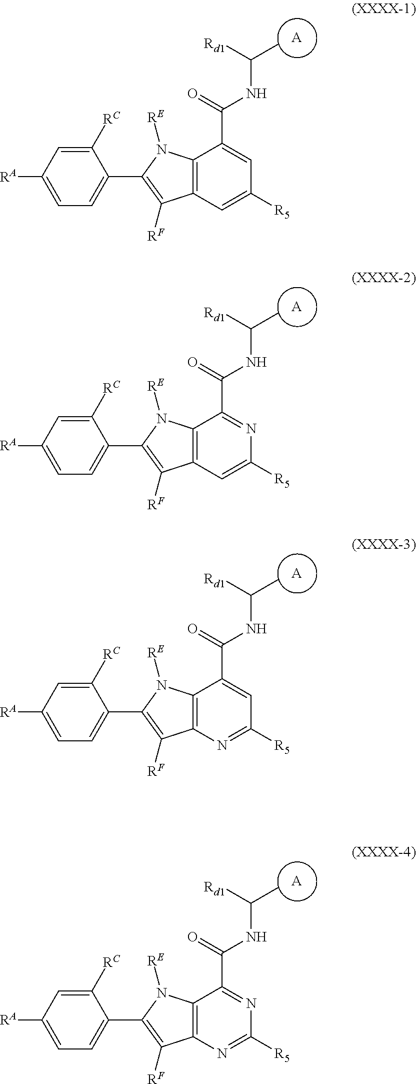

wherein R_{d1}, \textcircled{A}, R^{A}, R^{C}, R^{E}, R^{F}, and R₅ are as previously defined.

In certain embodiments of the present invention, the compound of Formula (I) is represented by one of Formulae (XXXXI-1)˜(XXXXI-4),

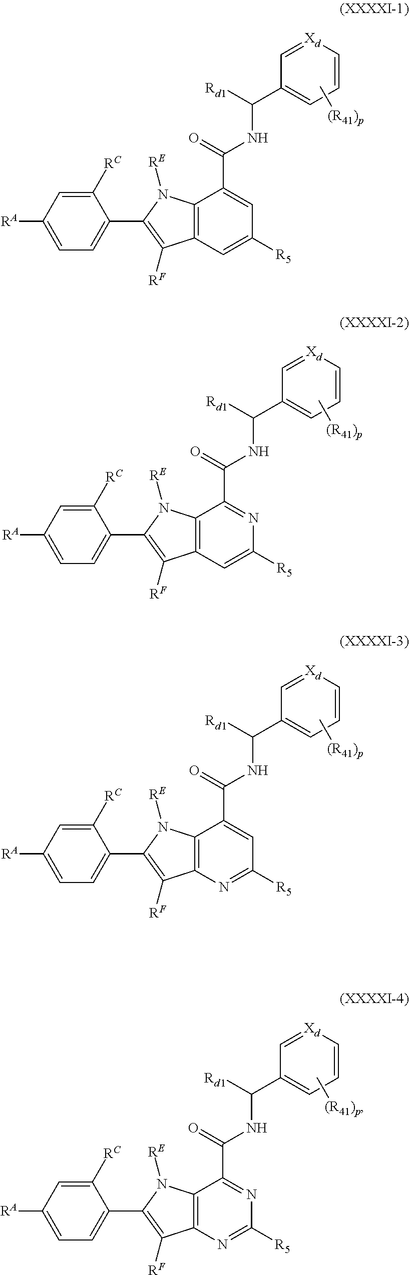

wherein X_{d} is N or CH; and R_{d1}, R₄₁, p, R^{A}, R^{C}, R^{E}, R^{F}, and R₅ are as previously defined. Preferably, R_{d1} is hydrogen or an optionally substituted methyl group.

In certain embodiments of the present invention, the compound of Formula (I) is represented by one of Formulae (XXXXII-1)˜(XXXXII-8),

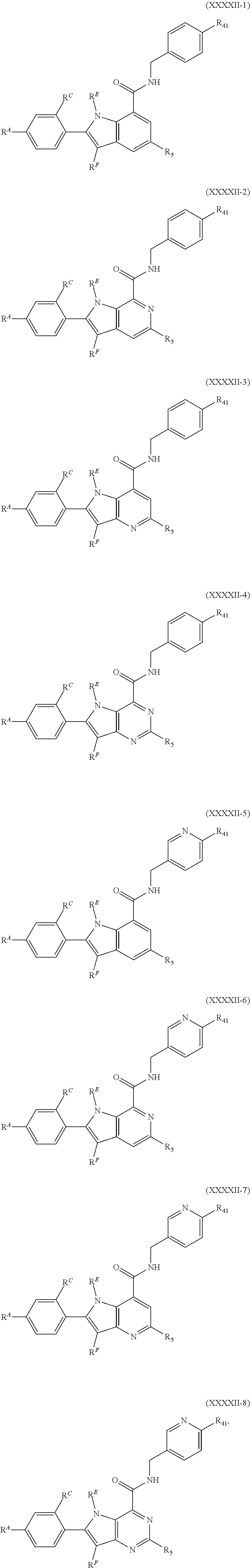

wherein R₄₁, R^{A}, R^{C}, R^{E}, R^{F}, and R₅ are as previously defined.

In certain embodiments of the present invention, the compound of Formula (I) is represented by one of Formulae (XXXXIII-1)˜(XXXXIII-8),

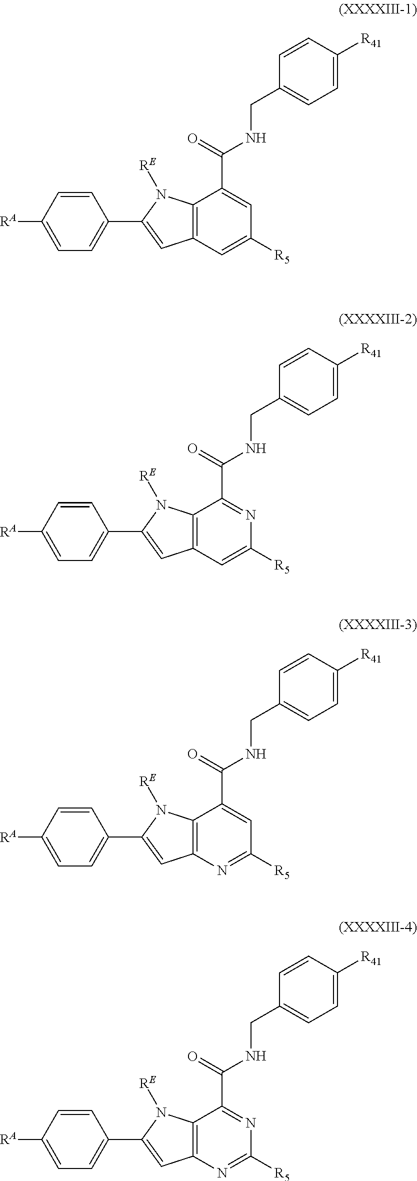

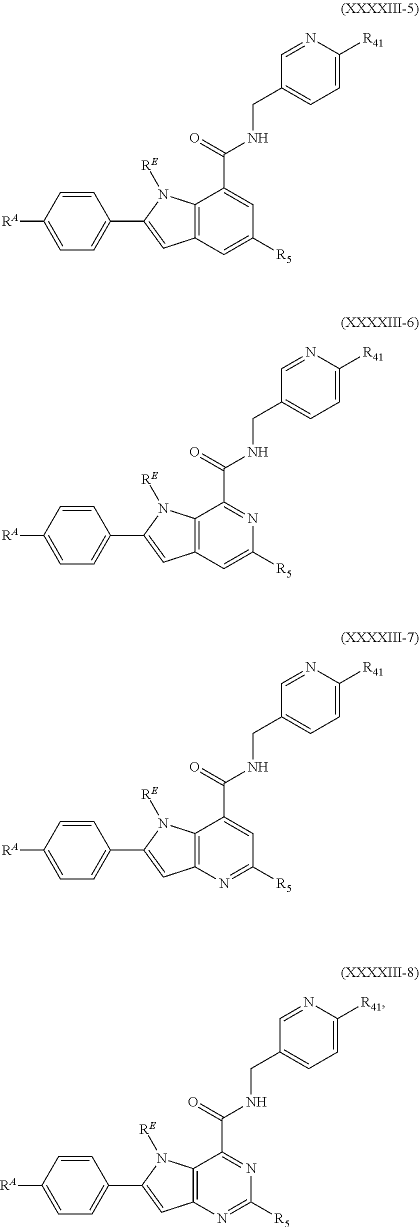

wherein R₄₁, R^{A}, R^{E}, and R₅ are as previously defined.

In certain embodiments of the present invention, the compound of Formula (I) is represented by one of Formulae (XXXVIII), (XXXIX), (XXXX-1)˜(XXXX-4), (XXXXI-1)˜(XXXXI-4), (XXXXII-1)˜(XXXXII-8), and (XXXXIII-1)˜(XXXXIII-8), wherein R^{A} is selected from:

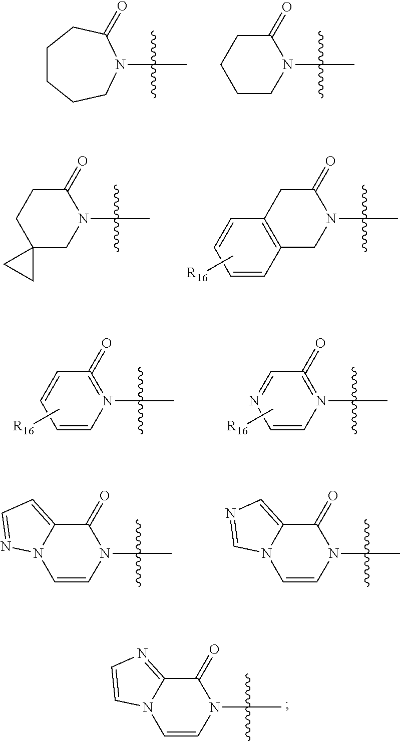

is selected from —C(O)N(R₁)(R₂), —NR₁R₂, —N(R₁)C(O)(R₂), —N(R₁)C(O)O(R₂), —N(R₁)S(O)₂(R₂), and —NR₁ₐC(O)NR₁R₂; R₁ₐ, R₁, R₂, and R₁₆ are as previously defined.

In certain embodiments of the present invention, the compound of Formula (I) is represented by one of Formulae (XXXVIII), (XXXIX), (XXXX-1)˜(XXXX-4), (XXXXI-1)˜(XXXXI-4), (XXXXII-1)˜(XXXXII-8), and (XXXXIII-1)˜(XXXXIII-8), wherein R^{A} is selected from:

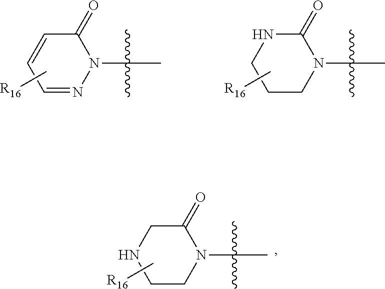

R₄₁ is selected from —C(O)N(R₁)(R₂), —NR₁R₂, —N(R₁)C(O)(R₂), —N(R₁)C(O)O(R₂), —N(R₁)S(O)₂(R₂), and —NR₁ₐC(O)NR₁R₂; R₁ₐ, R₁, R₂, and R₁₆ are as previously defined.

In certain embodiments of the present invention, the compound of Formula (I) is represented by one of Formulae (XXXXIV-1)˜(XXXXIV-8),

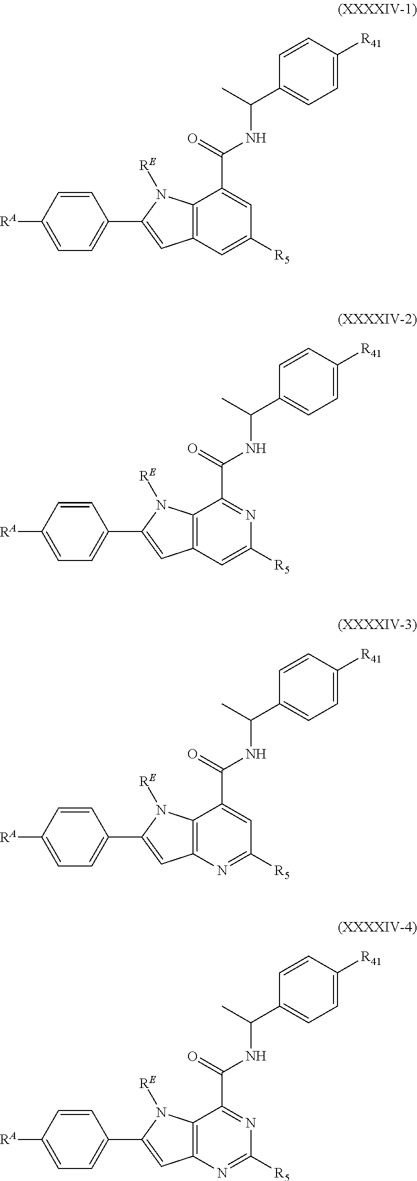

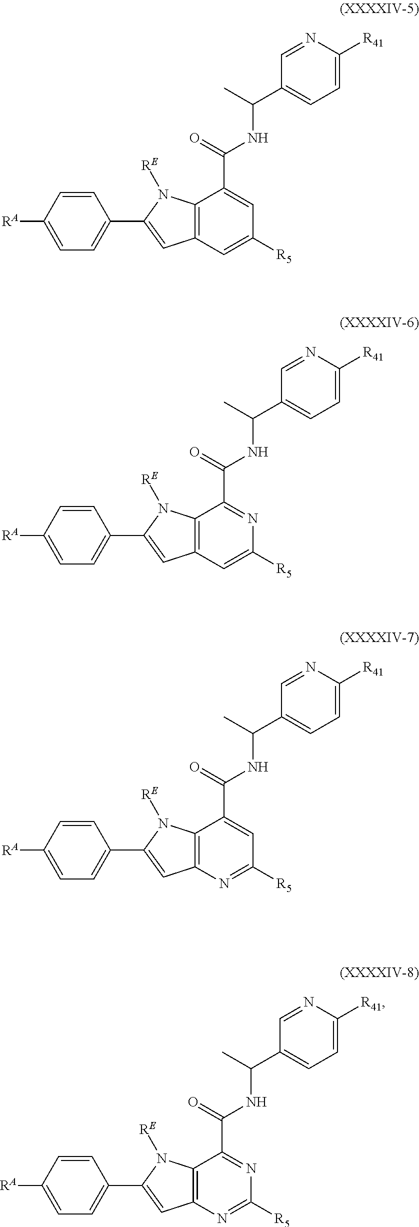

wherein R₄₁, R^{A}, R^{E}, and R₅ are as previously defined.

In certain embodiments of the present invention, the compound of Formula (I) is represented by one of Formulae (XXXXIV-1)˜(XXXXIV-8), wherein R_{A} is selected from:

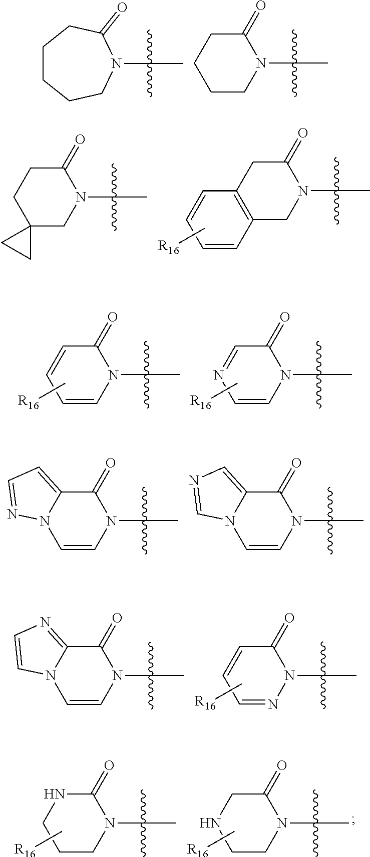

is selected from —C(O)N(R₁)(R₂), —NR₁R₂, —N(R₁)C(O)(R₂), —N(R₁)C(O)O(R₂), —N(R₁)S(O)₂(R₂), and —N₁ₐC(O)NR₁R₂; and R₁ₐ, R₁, R₂, and R₁₆ are as previously defined.

Each preferred group stated above can be taken in combination with one, any or all other preferred groups.

It will be appreciated that the description of the present invention herein should be construed in congruity with the laws and principles of chemical bonding. In some instances, it may be necessary to remove a hydrogen atom in order to accommodate a substitutent at any given location.

It will be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

It should be understood that the compounds encompassed by the present invention are those that are suitably stable for use as pharmaceutical agents.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4, or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8t, 2 to 12, 2 to 4, 3 to 4, or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups include, but are not limited to, methoxy, ethoxy, n-propoxy, 2-propoxy (isopropoxy) and the higher homologs and isomers. Preferred alkoxy is C₁-C₃ alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One skilled in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂-C₂-C₈-alkenyl, —OCO₂-C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, —CO₂—C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂-C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂—C₁-C₁₂-alkyl, —SO₂—C₂-C₈-alkenyl, —SO₂—C₂-C₈-alkynyl, —SO₂—C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents further include —C(O)NHSO₂—C₁-C₁₂-alkyl, —C(O)NHSO₂—C₂-C₈-alkenyl, —C(O)NHSO₂—C₂-C₈-alkynyl, —C(O)NHSO₂—C₃-C₁₂-cycloalkyl, —C(O)NHSO₂-aryl, —C(O)NHSO₂-heteroaryl, —C(O)NHSO₂-heterocycloalkyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, NJ (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable formulations.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Synthetic Methods

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

Non-limiting examples of synthetic schemes demonstrating the making of compounds of the invention are illustrated in Schemes 1-7

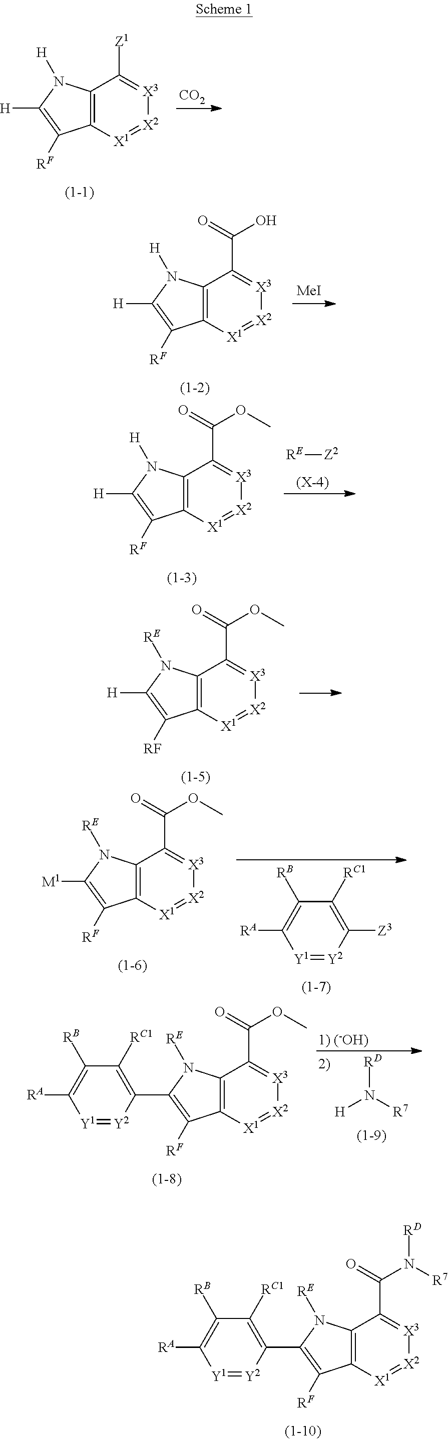

Scheme 1 illustrates a general method to prepare the compound of formula (1-10) from indolyl (pseudo)halide (1-1), wherein X¹, X², X³, R^{F1}, Y¹, Y², R^{A}, R^{B}, R^{C2}, R^{D}, and R⁷ are as previously described. Z¹, Z², and Z³ are suitably chosen halogen atoms or pseudo-halogen groups and M¹ represents B(OH)₂, BF₃K or B(OR)₂. The (pseudo)halide Z¹ of (1-1) is first activated using strong base (LDA, nBuLi, etc.) and then carboxylated using carbon dioxide. Alkylation of the resulting carboxylic acid (1-2) in the presence of base furnishes the aryl ester (1-3), depicted here as the methyl ester. Alkylation of the indole nitrogen with an alkyl (pseudo)halide (1-4) under basic conditions provides (1-5). Compound (1-5) is then reacted with a suitable M¹ source (B(EtO)₃, pinBOⁱPr, etc.) with a strong base to afford the cross-coupling partner (1-6), which is then reacted with the aryl or heteroaryl (pseudo)halide (1-7) under common palladium-catalyzed Suzuki reaction conditions. Finally, ester (1-8) is hydrolyzed using a hydroxide source (LiOH, NaOH, etc.) and the resulting carboxylic acid is reacted with an amine (1-9) under commonly employed amide coupling conditions (TCFH, HATU, EDC, etc.) to furnish compound (1-10).

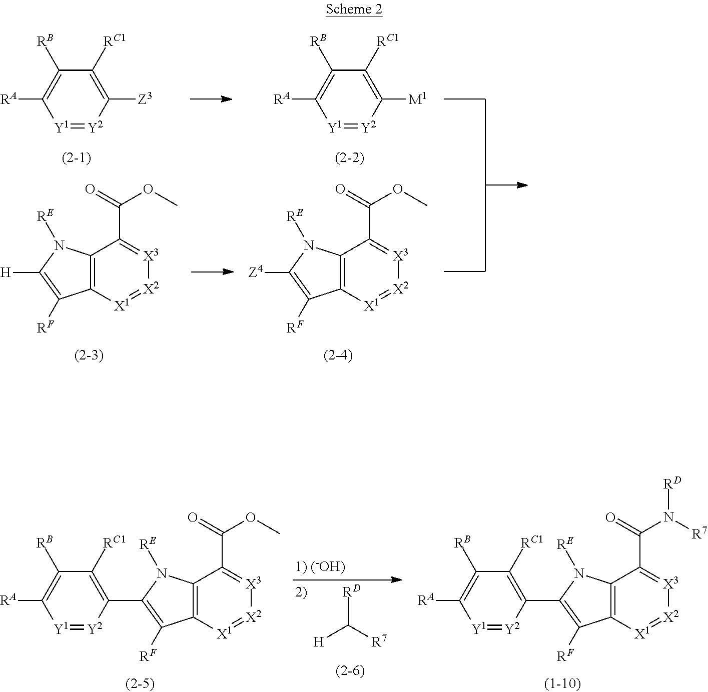

Scheme 2 provides an alternative method to prepare the compounds of formula (1-10), wherein Z⁴ is a suitably chosen halogen atom or pseudo-halogen group. Compound (2-1) is reacted with a suitable M¹ source (e.g. B₂pin₂) under typical transition metal-catalyzed conditions to afford compound (2-2). Indole (2-3) is reacted with a (pseudo)halide source (CBr₄, NBS, I₂) in the presence of a strong base (LDA) to provide indolyl (pseudo)halide (2-4). Compounds (2-2) and (2-4) are reacted under common palladium-catalyzed reaction conditions to provide ester (2-5). Ester (2-5) is hydrolyzed using a hydroxide source and the resulting carboxylic acid the reacted with an amine (2-6) under commonly employed amide coupling conditions to furnish compound (1-10).

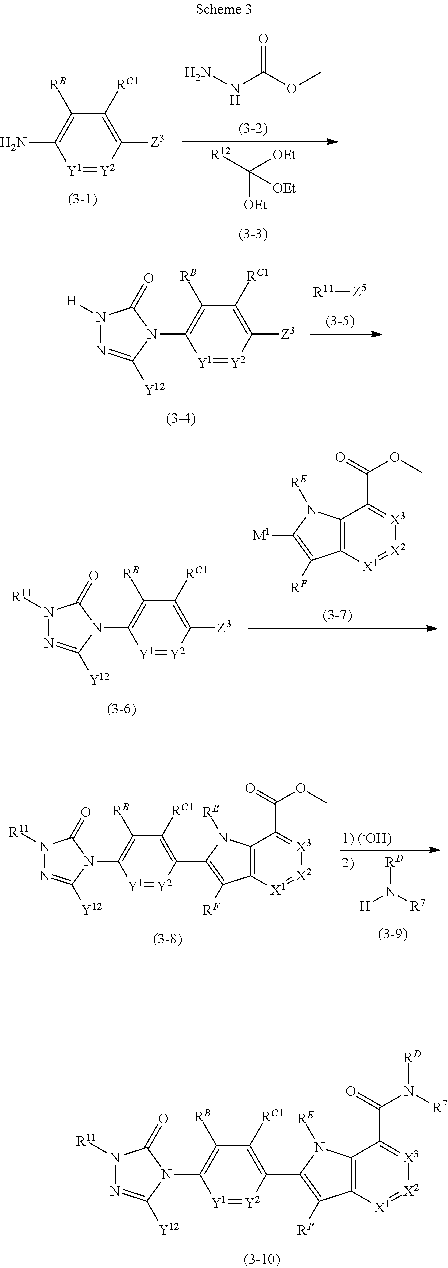

Scheme 3 depicts the synthesis of triazolone (3-10) from arene (3-1), wherein Z³ is a suitably chosen halogen atom or pseudo-halogen group. Aniline (3-1) is first treated with methyl hydrazinocarboxylate (3-2) and triethyl orthoacetate (3-3) to deliver triazolone (3-4). Compound (3-4) is alkylated with a suitable alkyl halide (3-5) to afford compound (3-6), which is then reacted with indole (3-7) under common palladium-catalyzed Suzuki reaction conditions. Finally, ester (3-8) is hydrolyzed using a hydroxide source and the resulting carboxylic acid is reacted with an amine (3-9) under commonly employed amide coupling conditions to furnish compound (3-10).

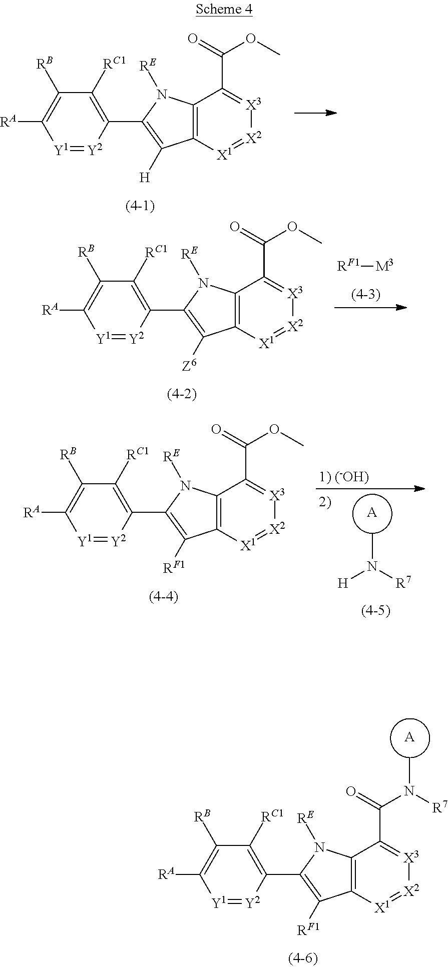

Scheme 4 depicts the synthesis of (4-6) from indole (4-1) wherein M³ represents B(OH)₂, BF₃K or B(OR)₂. Halogenation of indole (4-1) delivers compound (4-2), which is then reacted with coupling partner (4-3) under common palladium-catalyzed Suzuki reaction conditions to provide ester (4-4). Finally, ester (4-4) is hydrolyzed using a hydroxide source and the resulting carboxylic acid is reacted with an amine (4-5) under commonly employed amide coupling conditions to furnish compound (4-6).

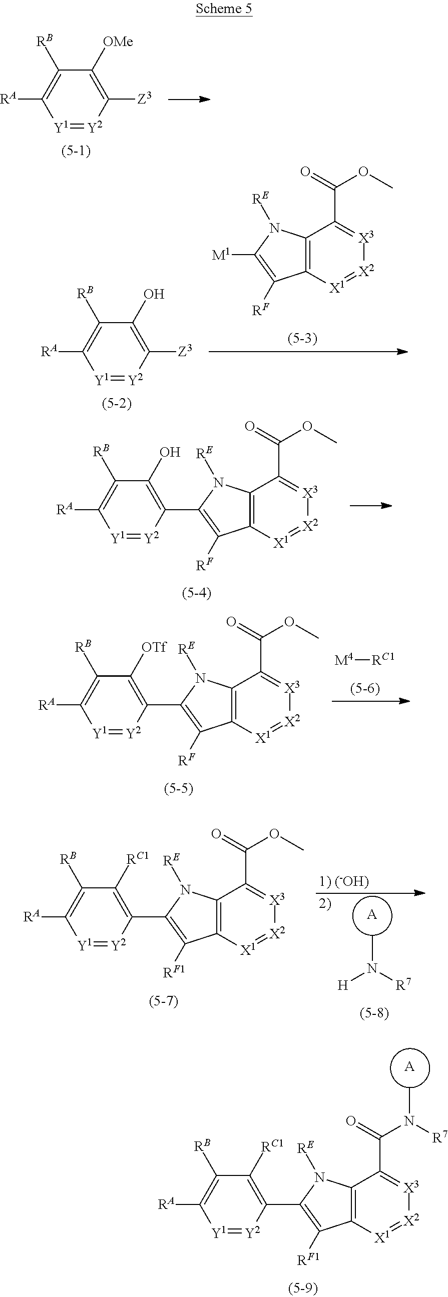

Scheme 5 depicts the synthesis of (5-9) from anisole (5-1) wherein M⁴ represents B(OH)₂, BF₃K or B(OR)₂, Zn, H. Anisole (5-1) is demethylated to phenol (5-2), which is then cross-coupled with indole (5-3). Compound (5-4) is protected as the triflate using reagents such as Tf₂O under basic conditions. Coupling of indole (5-5) with (5-6) delivers compound (5-7). Finally, ester (5-7) is hydrolyzed using a hydroxide source and the resulting carboxylic acid is reacted with an amine (5-8) under commonly employed amide coupling conditions to furnish compound (5-9).

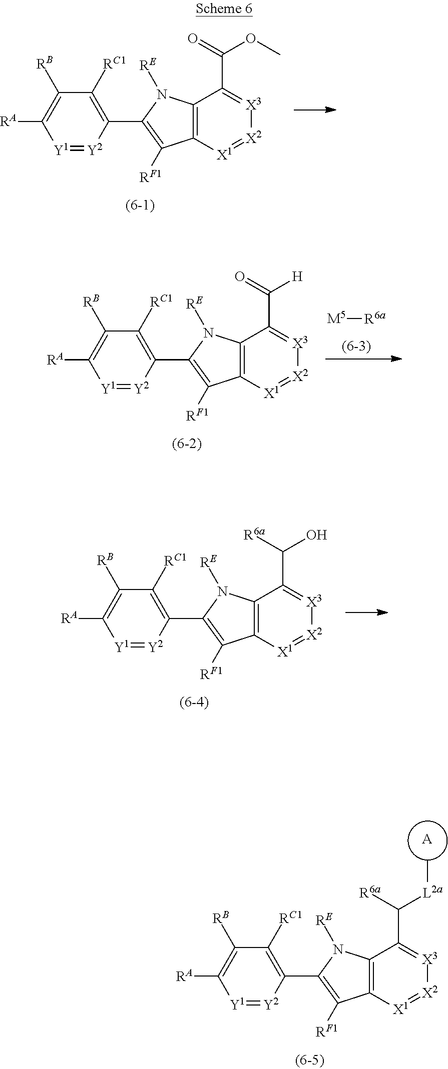

Scheme 6 depicts the synthesis of (6-6) from indole (6-1). Reduction of ester (6-1) using a metal hydride (DIBAL-H, etc.) delivers aldehyde (6-2), which is then treated with a nucleophile (6-3) to provide secondary alcohol (6-4). Group (A) is installed via Mitsunobu reaction or via nucleophilic aromatic substitution to provide compound (6-5).

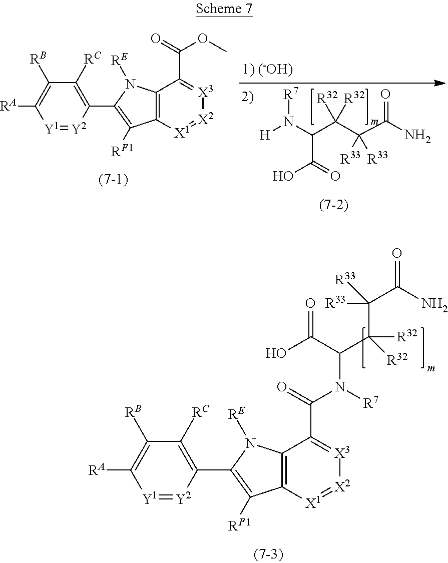

Scheme 7 depicts the synthesis of (7-3) from indole (7-1). Ester (7-1) is hydrolyzed using a hydroxide source and then treated under Schotten-Baumann coupling conditions (SOCl₂). The resulting acid chloride is treated with amino acid derivative (7-2) under basic conditions to deliver compound (7-3).

### EXAMPLES

The compounds and processes of the present invention will be better understood in connection with the following examples, which are intended as an illustration only and not limiting the scope of the invention. Starting materials were either available from a commercial vendor or produced by methods well known to those skilled in the art.

### General Conditions:

Mass spectra were run on LC-MS systems using electrospray ionization. These were Agilent 1290 Infinity II systems with an Agilent 6120 Quadrupole detector. Spectra were obtained using a ZORBAX Eclipse XDB-C18 column (4.6×30 mm, 1.8 micron). Spectra were obtained at 298K using a mobile phase of 0.1% formic acid in water (A) and 0.1% formic acid in acetonitrile (B). Spectra were obtained with the following solvent gradient: 5% (B) from 0-1.5 min, 5-95% (B) from 1.5-4.5 min, and 95% (B) from 4.5-6 min. The solvent flowrate was 1.2 mL/min. Compounds were detected at 210 nm and 254 nm wavelengths. [M+H]⁺ refers to mono-isotopic molecular weights.

NMR spectra were run on a Bruker 400 MHz spectrometer. Spectra were measured at 298K and referenced using the solvent peak. Chemical shifts for ¹H NMR were reported in parts per million (ppm).

Compounds were purified via reverse-phase high-performance liquid chromatography (RPHPLC) using a Gilson GX-281 automated liquid handling system. Compounds were purified on a Phenomenex Kinetex EVO C18 column (250×21.2 mm, 5 micron), unless otherwise specified. Compounds were purified at 298K using a mobile phase of water (A) and acetonitrile (B) using gradient elution between 0% and 100% (B), unless otherwise specified. The solvent flowrate was 20 mL/min and compounds were detected at 254 nm wavelength.

Alternatively, compounds were purified via normal-phase liquid chromatography (NPLC) using a Teledyne ISCO Combiflash purification system. Compounds were purified on a REDISEP silica gel cartridge. Compounds were purified at 298K and detected at 254 nm wavelength.

### Ex. 1: Synthesis of N-(3-carbamoyl-2-fluorophenyl)-2-(4-(1,3-dimethyl-5-oxo-1,5-dihydro-4H-1,2,4-triazol-4-yl)phenyl)-1-(2-methoxyethyl)-3-methyl-5-(trifluoromethyl)-1H-indole-7-carboxamide

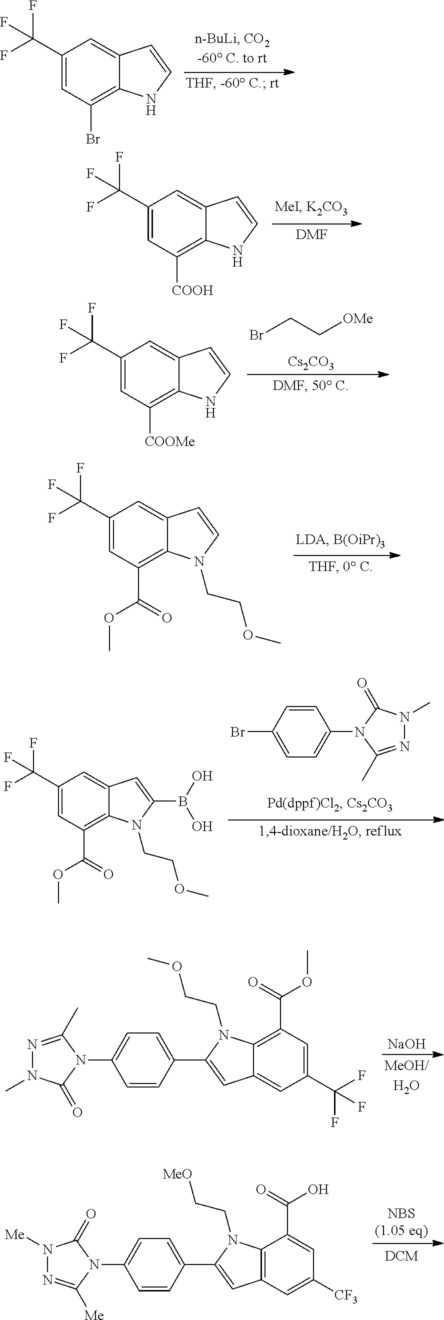

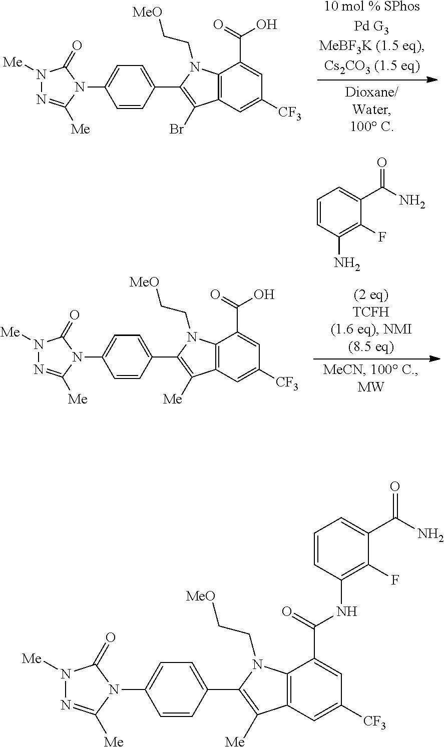 [SMILES: COCCn1c(-c2ccc(-n3c(C)nn(C)c3=O)cc2)c(Br)c2cc(C(F)(F)F)cc(C(=O)O)c21 ; "10 mol % SPhosPd G3MeBF3K (1.5 eq),Cs2CO3 (1.5 eq)" ; NC(=O)c1cccc(N)c1F ; COCCn1c(-c2ccc(-n3c(C)nn(C)c3=O)cc2)c(C)c2cc(C(F)(F)F)cc(C(=O)O)c21 ; COCCn1c(-c2ccc(-n3c(C)nn(C)c3=O)cc2)c(C)c2cc(C(F)(F)F)cc(C(=O)Nc3cccc(C(N)=O)c3F)c21]

### Step 1

To a solution of 7-bromo-5-(trifluoromethyl)-1H-indole (19.5 g, 73.9 mmol, 1.0 eq) in THF (400 mL) was added n-BuLi (2.5 N, 88.4 mL, 221.0 mmol, 3.0 eq) dropwise at −60° C. under a nitrogen atmosphere. The mixture was warmed to 0° C. and stirred for 0.5 h, then the mixture was cooled to −60° C., and copious amounts of dry ice were added. Upon complete addition of the dry ice, the ice bath was removed, and the mixture was warmed to room temperature and stirred for 10 min. The reaction was quenched by water (400 mL) and extracted with EtOAc (400 mL). The aqueous phase was acidified with 1 N HCl and extracted with EtOAc (300 mL×2). The organic layers were dried over Na₂SO₄, filtered, and concentrated to give 5-(trifluoromethyl)-1H-indole-7-carboxylic acid (11.8 g, 65% yield) as a yellow solid. ¹H NMR (400 MHz, CDCl3): δ 13.46 (brs, 1H), 11.52 (s, 1H), 8.24 (s, 1H), 7.96 (s, 1H), 7.55 (t, J=2.8 Hz, 1H), 6.74 (t, J=2.0 Hz, 1H). ESI MS m/z=228.0 [M+H]⁺.

### Step 2

To a solution of 5-(trifluoromethyl)-1H-indole-7-carboxylic acid (11.8 g, 51.5 mmol, 1.0 eq) in DMF (200 mL) was added K₂CO₃ (28.5 g, 206.0 mmol, 4.0 eq) and Mel (21.9 g, 154.5 mmol, 3.0 eq) at room temperature. The mixture was stirred for 2 h, then poured into water (300 mL). The solid was filtered and dried to give methyl 5-(trifluoromethyl)-1H-indole-7-carboxylate (10.8 g, 86% yield) as a light brown solid. ¹H NMR (300 MHz, CDCl3): δ 11.63 (s, 1H), 8.28 (s, 1H), 7.98 (s, 1H), 7.61 (d, J=3.3 Hz, 1H), 6.77 (d, J=3.3 Hz, 1H), 3.98 (s, 3H). m/z=242.1 [M+H]⁺.

### Step 3

To a solution of methyl 5-(trifluoromethyl)-1H-indole-7-carboxylate (3.0 g, 12.3 mmol, 1.0 eq) in DMF (60 mL) was added Cs₂CO₃ (12.1 g, 37.0 mmol, 3.0 eq) and 1-bromo-2-methoxyethane (5.1 g, 37.0 mmol, 3.0 eq) at room temperature. The resulting mixture was stirred at 50° C. for 24 h, then it was poured into water (120 mL) and extracted with EtOAc (100 mL×2). The combined organic layers were washed with brine (80 mL×3), dried over Na₂SO₄, concentrated and the residue was purified by column chromatography (petroleum ether/EtOAc=20/1) to give methyl 1-(2-methoxyethyl)-5-(trifluoromethyl)-1H-indole-7-carboxylate (3.5 g, 94% yield) as a light colorless oil. ¹H NMR (300 MHz, CDCl3): δ 8.04 (s, 1H), 7.91 (s, 1H), 7.28 (d, J=3.3 Hz, 1H), 6.66 (d, J=3.3 Hz, 1H), 4.59 (t, J=5.4 Hz, 2H), 3.98 (s, 3H), 3.64 (t, J=5.4 Hz, 2H), 3.24 (s, 3H).

### Step 4

To a solution of methyl 1-(2-methoxyethyl)-5-(trifluoromethyl)-1H-indole-7-carboxylate (1.5 g, 5.0 mmol, 1.0 eq) in THF (30 mL) was added triisopropyl borate (1.5 g, 8.0 mmol, 1.6 eq). The resulting solution was cooled to 0° C. LDA (1 N, 6.0 mmol, 1.2 eq) was then added dropwise. The mixture was stirred for 3 h, quenched with water (50 mL), and extracted with EtOAc (40 mL). The aqueous layer was acidified with 1 N HCl and extracted with EtOAc (50 mL×2). The combined organic layers were concentrated to give (7-(methoxycarbonyl)-1-(2-methoxyethyl)-5-(trifluoromethyl)-1H-indol-2-yl)boronic acid (1.1 g, crude yield 64%) as a yellow solid. ESI MS m/z=346.1 [M+H]⁺.

### Step 5

To a solution of (7-(methoxycarbonyl)-1-(2-methoxyethyl)-5-(trifluoromethyl)-1H-indol-2-yl)boronic acid (1.2 g, 4.3 mmol, 1.0 eq) and 4-(4-bromophenyl)-2,5-dimethyl-2,4-dihydro-3H-1,2,4-triazol-3-one (1.5 g, 4.3 mmol, 1.0 eq) were added Cs₂CO₃ (2.8 g, 8.7 mmol, 2.0 eq) and Pd(dppf)Cl₂ (318 mg, 0.43 mmol, 0.1 eq) under a nitrogen atmosphere. The mixture was heated to reflux and stirred overnight, then the mixture was cooled and poured into water (50 mL) and extracted with EtOAc (50 mL). The organic layer was concentrated and the residue was purified by silica gel column chromatography (100% EtOAc) to give methyl 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylate (0.75 g, 35% yield) as a yellow solid. 1H NMR (300 MHz, DMSO-d6): δ 8.26 (s, 1H), 7.81 (d, J=1.5 Hz, 1H), 7.73 (d, J=8.4 Hz, 2H), 7.61 (d, J=8.4 Hz, 2H), 6.95 (s, 1H), 4.58 (t, J=5.1 Hz, 2H), 3.96 (s, 3H), 3.38-3.34 (m, 4H), 2.89 (s, 3H), 2.16 (s, 3H). ESI MS m/z=489.1 [M+H]⁺.

### Step 6

To a solution of methyl 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylate (0.75 g, 1.54 mmol, 1.0 eq) in MeOH (8 mL) was added aq. NaOH (10%, 8 mL), the mixture was stirred for 2 h and heated at 60° C. for 2 h, then it was cooled and acidified with 2 N HCl. The solid was filtered and washed with H₂O (20 mL) and dried to give 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (0.64 g, 87% yield) as a brown solid. 1H NMR (400 MHz, DMSO-d6): δ 13.66 (brs, 1H), 8.22 (s, 1H), 7.81 (s, 1H), 7.74 (d, J=8.4 Hz, 2H), 7.61 (d, J=8.4 Hz, 2H), 6.93 (s, 1H), 4.66 (t, J=5.2 Hz, 2H), 3.40-3.32 (m, 4H), 2.88 (s, 3H), 2.16 (s, 3H). ESI MS m/z=475.1[M+H]⁺.

### Step 7

To a solution of 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (102 mg, 349 μmol) in DCM (5 mL), was added N-bromosuccinimide (156 mg, 365 μmol). The reaction was stirred at room temperature for 4 hours, then quenched with Na₂SO₃ and water. The aqueous phase was extracted with DCM. The combined organic layer was filtered through a plug of silica gel and concentrated to provide 3-bromo-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (201 mg, 349 μmol, 81% yield). ESI MS m/z=553.1 [M+H]⁺.

### Step 8

To a vial containing 3-bromo-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (50 mg, 90 μmol), potassium trifluoro(methyl)borate (27 mg, 2 eq, 0.18 mmol), Cs₂CO₃ (0.15 g, 5 Eq, 0.45 mmol), and SPhos Pd G3 (7.9 mg, 9.0 μmol) was added 1,4-dioxane (0.30 mL) and water (0.15 mL). The reaction was purged with nitrogen gas, heated to 100° C., and monitored for completion by LCMS. The reaction was concentrated and directly subjected to column chromatography to provide 2-(4-(1,3-dimethyl-5-oxo-1,5-dihydro-4H-1,2,4-triazol-4-yl)phenyl)-1-(2-methoxyethyl)-3-methyl-5-(trifluoromethyl)-1H-indole-7-carboxylic acid (21 mg, 43 μmol, 48% yield). ESI MS m/z=489.3 [M+H]⁺.

### Step 9

To a microwave vial containing 2-(4-(1,3-dimethyl-5-oxo-1,5-dihydro-4H-1,2,4-triazol-4-yl)phenyl)-1-(2-methoxyethyl)-3-methyl-5-(trifluoromethyl)-1H-indole-7-carboxylic acid (21 mg, 43 μmol) was added acetonitrile (0.43 mL), 3-amino-2-fluorobenzamide (13 mg, 86 μmol), followed by TCFH (18 mg, 1.5 Eq, 43 μmol) and NMI (30 mg, 29 μL, 0.37 mmol). The reaction mixture was heated at 100° C. in microwave reactor for 30 min and monitored by LCMS. Upon completion the mixture was directly subjected to HPLC purification to provide N-(3-carbamoyl-2-fluorophenyl)-2-(4-(1,3-dimethyl-5-oxo-1,5-dihydro-4H-1,2,4-triazol-4-yl)phenyl)-1-(2-methoxyethyl)-3-methyl-5-(trifluoromethyl)-1H-indole-7-carboxamide (6 mg, 43 μmol, 20% yield). ESI MS m/z=625.4 [M+H]⁺.

The following examples were prepared using procedures similar to those described in Ex. 1:

| Example | Structure | ESI-MS |
|---|---|---|
| 2 | 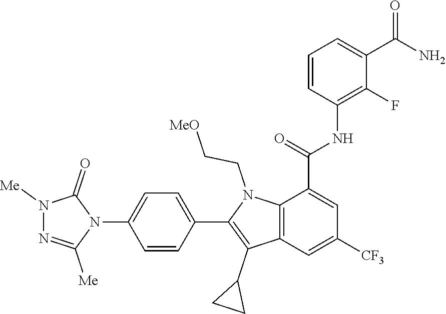 [SMILES: COCCn1c(-c2ccc(-n3c(C)nn(C)c3=O)cc2)c(C2CC2)c2cc(C(F)(F)F)cc(C(=O)Nc3cccc(C(N)=O)c3F)c21] | [M + H]⁺ 651.1 |
| 3 | 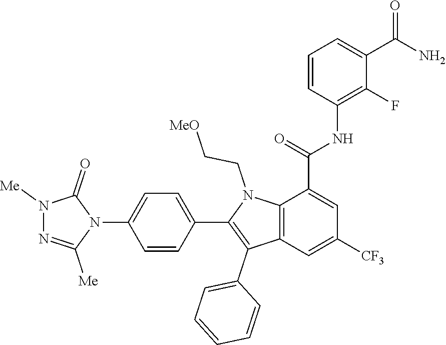 [SMILES: COCCn1c(-c2ccc(-n3c(C)nn(C)c3=O)cc2)c(-c2ccccc2)c2cc(C(F)(F)F)cc(C(=O)Nc3cccc(C(N)=O)c3F)c21] | [M + H]⁺ 687.1 |

### Ex. 4: Synthesis of N-(3-carbamoyl-2-fluorophenyl)-2-(4-(1,3-dimethyl-5-oxo-1,5-dihydro-4H-1,2,4-triazol-4-yl)-2-vinylphenyl)-1-(2-methoxyethyl)-5-(trifluoromethyl)-1H-indole-7-carboxamide

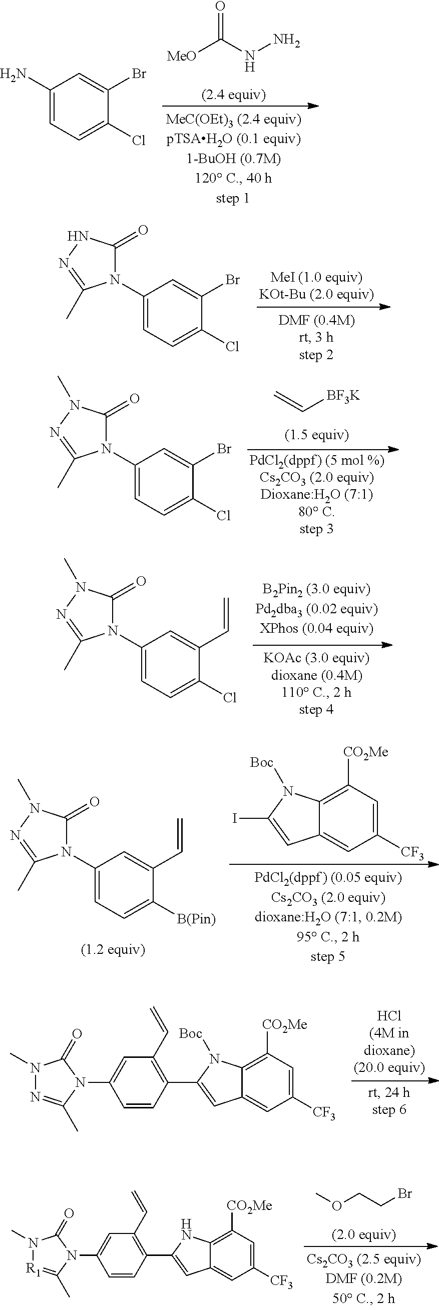

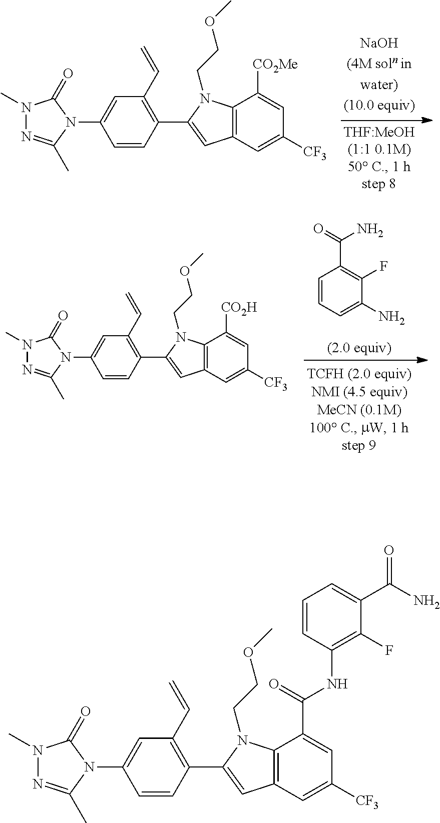 [SMILES: C=Cc1cc(-n2c(C)nn(C)c2=O)ccc1-c1cc2cc(C(F)(F)F)cc(C(=O)OC)c2n1CCOC ; C=Cc1cc(-n2c(C)nn(C)c2=O)ccc1-c1cc2cc(C(F)(F)F)cc(C(=O)O)c2n1CCOC ; NC(=O)c1cccc(N)c1F ; C=Cc1cc(-n2c(C)nn(C)c2=O)ccc1-c1cc2cc(C(F)(F)F)cc(C(=O)Nc3cccc(C(N)=O)c3F)c2n1CCOC]

### Step 1

In a 250 mL round bottom flask equipped with a stir bar, 3-bromo-4-chloro aniline (3.0 g, 1.0 equiv., CAS #: 823-54-1), methyl hydrazinecarboxylate (1.57 g, 1.2 equiv., CAS #: 6294-89-9), 1,1,1-triethoxyethane (3.20 mL, 1.2 equiv., CAS #: 78-39-7) and pTSA·H₂O (138 mg, 0.05 equiv., CAS #: 6192-52-5) were combined neat, followed by addition of 1-BuOH (20.8 mL). The reaction mixture was heated at 120° C. on a heating block. Reaction progress was monitored using LC-MS. After 16 h, additional 1,1,1-triethoxyethane (3.2 mL, 1.2 equiv.), methyl hydrazinecarboxylate (1.57 g, 1.2 equiv.) and pTSA·H₂O (138 mg, 0.05 equiv.) were added, and the reaction mixture was stirred for additional 24 h at 120° C. After completion, 1-BuOH was removed under vacuum. The crude residue was redissolved in 100 mL ethyl acetate and washed with water (4×, 100 mL). Then the organic layer was dried over sodium sulfate and concentrated under vacuum. The resultant solid was stirred in 40 mL diethyl ether for 20 min and left in the fridge overnight. Then the solid product was collected by filtration and washed with cold diethyl ether and followed by dried under vacuum to afford 4-(3-bromo-4-chlorophenyl)-5-methyl-2,4-dihydro-3H-1,2,4-triazol-3-one (1.3 g, 31% yield). ESI MS m/z=289.9 [M+H]⁺. Material was transferred to the next reaction without any further purification.

### Step 2

In a 40 mL vial equipped with a stir bar, 4-(3-bromo-4-chlorophenyl)-5-methyl-2,4-dihydro-3H-1,2,4-triazol-3-one (1.3 g, 1.0 equiv.) was dissolved in dry DMF (11 mL) at room temperature, followed by the addition of potassium tert-butoxide (1.0 g, 2.0 equiv.) and dropwise addition of methyl iodide (0.28 mL, 1.0 equiv.). The reaction mixture was stirred at room temperature for 3 h. Reaction progress was monitored using LC-MS. Once completed, 50 mL water was added to the reaction mixture. The resulting solid was collected by filtration, washed with water, and dried under vacuum to afford 4-(3-bromo-4-chlorophenyl)-2,5-dimethyl-2,4-dihydro-3H-1,2,4-triazol-3-one (1.2 g, 88% yield). ESI MS m/z=303.9 [M+H]⁺. Material was transferred to the next reaction without any further purification.

### Step 3

In a 40 mL vial equipped with a stir bar, 4-(3-bromo-4-chlorophenyl)-2,5-dimethyl-2,4-dihydro-3H-1,2,4-triazol-3-one (1.2 g, 1.0 equiv.), potassium vinyltrifluoroborate (531 mg, 1.0 equiv.), [1,1′-Bis(diphenylphosphino)ferrocene]dichloropalladium(II) (145 mg, 0.05 equiv.), and cesium carbonate (2.58 g, 2.0 equiv.) were combined neat and followed by dissolved in 1,4-dioxane (17.4 mL) and water (2.48 mL). The reaction vial was purged with N₂ and heated to 90° C. in a heating block. The reaction progress was monitored using LC-MS. Upon complete conversion, the reaction mixture was concentrated under vacuum and purified by silica gel column chromatography to afford 4-(4-chloro-3-vinylphenyl)-2,5-dimethyl-2,4-dihydro-3H-1,2,4-triazol-3-one (700 mg, 71% yield). ESI MS m/z=250.2 [M+H]⁺.

### Step 4

In a 20 mL vial equipped with a stir bar, 4-(4-chloro-3-vinylphenyl)-2,5-dimethyl-2,4-dihydro-3H-1,2,4-triazol-3-one (200 mg, 1.0 equiv.), bis(pinacolato)diboron (610 mg, 3.0 equiv.) Tris(dibenzylideneacetone)dipalladium (14.7 mg, 0.02 equiv., CAS #: 51364-51-3), X-Phos (15.3 mg, 0.04 equiv., CAS #: 564483-18-7), and potassium acetate (236 mg, 3.0 equiv.) were combined neat under nitrogen atmosphere, followed by addition of dry 1,4-dioxane (2 mL). Reaction mixture was evacuated and backfilled with nitrogen 5 times. Then the reaction mixture was heated to 110° C. in a heating block for 2 h. Reaction progress was monitored using LC-MS. After cooling to the room temperature, the reaction mixture was filtered through a thin pad of celite (eluting with ethyl acetate) and the eluent was concentrated under reduced pressure. The crude residue was purified by silica gel column chromatography (0 to 10% methanol/dichloromethane) to afford 2,5-dimethyl-4-(4-(4,4,5,5-tetramethyl-1,3,2-dioxaborolan-2-yl)-3-vinylphenyl)-2,4-dihydro-3H-1,2,4-triazol-3-one (280 mg, 92% yield). ESI MS m/z=342.0 [M+H]⁺.

### Step 5

In a 40 mL vial equipped with a stir bar, 1-(tert-butyl) 7-methyl 2-iodo-5-(trifluoromethyl)-1H-indole-1,7-dicarboxylate (650 mg, 1.0 equiv.), 2,5-dimethyl-4-(4-(4,4,5,5-tetramethyl-1,3,2-dioxaborolan-2-yl)-3-vinylphenyl)-2,4-dihydro-3H-1,2,4-triazol-3-one (567 mg, 1.2 equiv.), [1,1′-bis(diphenylphosphino)ferrocene]dichloropalladium(II) (50.7 mg, 0.05 equiv., CAS #: 72287-26-4) and cesium carbonate (1.15 g, 2.5 equiv., CAS #: 534-17-8) were combined neat under nitrogen atmosphere and followed by addition of 1,4-dioxane (6.0 mL) and water (0.9 mL). The reaction mixture was heated at 95° C. for 2 h in a heating block. Reaction progress was monitored using LC-MS. Once completed, the reaction mixture was diluted with ethyl acetate and washed with water. The aqueous layer was further extracted with ethyl acetate (×3). Then, the combined organic layer was washed with brine (×2), water (×2) and dried over sodium sulfate and concentrated under vacuum. The crude residue was purified by silica gel column chromatography (0 to 10% methanol/dichloromethane) to afford 1-(tert-butyl) 7-methyl 2-(4-(3,5-dimethyl-2-oxo-2,3-dihydro-1H-imidazol-1-yl)-2-vinylphenyl)-5-(trifluoromethyl)-1H-indole-1,7-dicarboxylate. ESI MS m/z=557.3 [M+H]⁺.

### Step 6

In a 40 mL vial equipped with a stir bar, 1-(tert-butyl) 7-methyl 2-(4-(1,3-dimethyl-5-oxo-1,5-dihydro-4H-1,2,4-triazol-4-yl)-2-vinylphenyl)-5-(trifluoromethyl)-1H-indole-1,7-dicarboxylate (380 mg, 1.0 equiv.) was taken and added HCl (3.4 mL, 20.0 equiv., 4M solution in Dioxane, CAS #7647-01-0). Reaction mixture was stirred for 24 h at room temperature. The crude residue was concentrated under vacuum to afford methyl 2-(4-(1,3-dimethyl-5-oxo-1,5-dihydro-4H-1,2,4-triazol-4-yl)-2-vinylphenyl)-5-(trifluoromethyl)-1H-indole-7-carboxylate. ESI MS m/z=457.1 [M+H]⁺. The crude was transferred to the next reaction without any further purification.

### Step 7

In a 8 mL vial equipped with a stir bar, methyl 2-(4-(3,5-dimethyl-2-oxo-2,3-dihydro-1H-imidazol-1-yl)-2-vinylphenyl)-5-(trifluoromethyl)-1H-indole-7-carboxylate (78 mg, 1.0 equiv.) was dissolved in dry DMF (0.85 mL) at room temperature, followed by the addition of cesium carbonate (140 mg, 2.5 equiv.) and dropwise 1-bromo-2-methoxyethane (34 μL, 2 equiv., CAS #: 6482-24-2). Reaction mixture was stirred at 50° C. in a heating block for 2 h. Reaction progress was monitored using LC-MS. Once completed, the reaction mixture was quenched with 1N HCl (aq) and diluted with ethyl acetate and layers were separated. The aqueous layer was further extracted with ethyl acetate (×3). Then, the combined organic layer was washed with brine (×2), water (×2) and dried over sodium sulfate and concentrated under vacuum. The crude residue was purified by silica gel column chromatography (0 to 10% methanol/dichloromethane) to afford methyl 2-(4-(1,3-dimethyl-5-oxo-1,5-dihydro-4H-1,2,4-triazol-4-yl)-2-vinylphenyl)-1-(2-methoxyethyl)-5-(trifluoromethyl)-1H-indole-7-carboxylate (42 mg, 48% yield). ESI MS m/z=515.3 [M+H]⁺.

### Step 8

In a 8 mL vial equipped with a stir bar, methyl 2-(4-(1,3-dimethyl-5-oxo-1,5-dihydro-4H-1,2,4-triazol-4-yl)-2-vinylphenyl)-1-(2-methoxyethyl)-5-(trifluoromethyl)-1H-indole-7-carboxylate (42 mg, 1.0 equiv.) was dissolved in THF (0.4 mL) and MeOH (0.4 mL), followed by addition of sodium hydroxide (0.2 mL, 10.0 equiv., 4M solution in water). The reaction mixture was stirred at 50° C. in a heating block for 1 h. Reaction progress was monitored using LC-MS. After completion, the reaction mixture was quenched by 1N HCl. Then the crude mixture was diluted with water and extracted with ethyl acetate (×3). Combined organic layer was washed with brine (×1), water (×1) and dried over sodium sulfate and concentrated under vacuum to afford 2-(4-(1,3-dimethyl-5-oxo-1,5-dihydro-4H-1,2,4-triazol-4-yl)-2-vinylphenyl)-1-(2-methoxyethyl)-5-(trifluoromethyl)-1H-indole-7-carboxylic acid. ESI MS m/z=501.2 [M+H]⁺. The crude was transferred to the next reaction without any further purification.

### Step 9

In a 2 mL microwave reaction vial equipped with a stir bar, 2-(4-(1,3-dimethyl-5-oxo-1,5-dihydro-4H-1,2,4-triazol-4-yl)-2-vinylphenyl)-1-(2-methoxyethyl)-5-(trifluoromethyl)-1H-indole-7-carboxylic acid (37 mg, 1.0 equiv.), 3-amino-2-fluorobenzamide (23 mg, 2.0 equiv., CAS #: 1369948-83-3) and TCFH (41 mg, 2.0 equiv., CAS #: 94790-35-9) were combined neat and followed by addition of dry acetonitrile (0.74 mL) at room temperature. Then, 1-methyl-1H-imidazole (27 μL, 4.5 equiv., CAS #: 616-47-7) was added and the reaction mixture was stirred under microwave condition for 60 min at 100° C. The crude reaction mixture was concentrated under vacuum and purified by silica gel column chromatography (0 to 10% methanol/dichloromethane). Isolated material was redissolved in 2.0 mL of dimethyl sulfoxide and repurified through RPHPLC to afford N-(3-carbamoyl-2-fluorophenyl)-2-(4-(1,3-dimethyl-5-oxo-1,5-dihydro-4H-1,2,4-triazol-4-yl)-2-vinylphenyl)-1-(2-methoxyethyl)-5-(trifluoromethyl)-1H-indole-7-carboxamide (10.1 mg, 21% yield). ¹H NMR (500 MHz, DMSO-d6) δ 10.77 (s, 1H), 8.22 (s, 1H), 7.90 (d, J=2.1 Hz, 1H), 7.84 (td, J=7.6, 1.8 Hz, 1H), 7.80 (s, 1H), 7.71 (d, J=1.8 Hz, 1H), 7.66 (s, 1H), 7.57 (d, J=8.1 Hz, 1H), 7.54-7.46 (m, 2H), 7.30 (t, J=7.9 Hz, 1H), 6.82 (s, 1H), 6.48 (dd, J=17.6, 11.2 Hz, 1H), 5.94 (d, J=17.4 Hz, 1H), 5.34 (d, J=11.3 Hz, 1H), 4.56-4.00 (m, 2H), 3.40-3.34 (m, 2H), 3.37 (s, 3H), 2.85 (s, 3H), 2.17 (s, 3H). ESI MS m/z=637.3 [M+H]⁺.

The following examples were prepared using procedures similar to those described in Ex. 4:

| Example | Structure | ESI-MS |
|---|---|---|
| 5 | 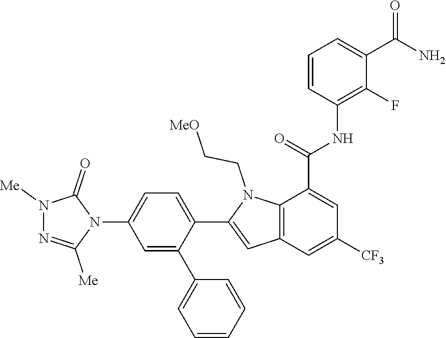 [SMILES: COCCn1c(-c2ccc(-n3c(C)nn(C)c3=O)cc2-c2ccccc2)cc2cc(C(F)(F)F)cc(C(=O)Nc3cccc(C(N)=O)c3F)c21] | [M + H]⁺ 687.1 |

### Ex. 6: Synthesis of N-(2-amino-2-oxoethyl)-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide

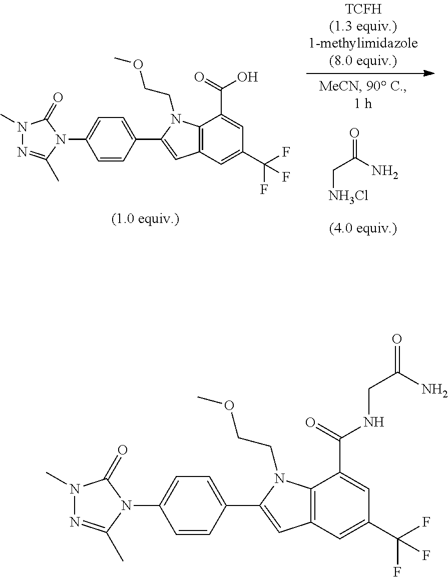

To a vial with a stir bar was added 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (23.0 mg, 0.0485 mmol, 1.0 equiv.), and TCFH (18.0 mg, 0.064 mmol, 1.3 equiv.). MeCN (0.323 mL) was added followed by the addition of 2-aminoacetamide hydrochloride 3 (21.4 mg, 0.194 mmol, 4.0 equiv.) and 1-methylimidazole (0.0309 mL, 0.388 mmol, 8.0 equiv.). The vial was sealed and heated to 90° C. with stirring for 1 hour. After this time, the reaction mixture was cooled to room temperature, diluted with water and EtOAc, and concentrated. The crude residue was purified by reverse-phase HPLC (10-100% MeCN in H₂O+0.1% TFA) to afford the pure product N-(2-amino-2-oxoethyl)-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide (8.2 mg, 32%) as a white solid. ESI-MS m/z=531.3 [M+H]⁺.

The following examples were prepared using procedures similar to those described in Ex. 3:

| Example | Structure | ESI-MS |
|---|---|---|
| 7 | 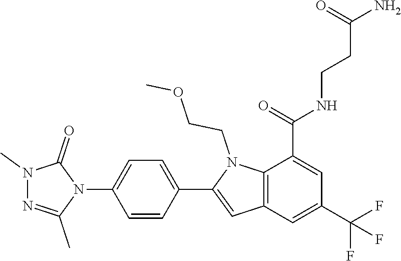 [SMILES: COCCn1c(-c2ccc(-n3c(C)nn(C)c3=O)cc2)cc2cc(C(F)(F)F)cc(C(=O)NCCC(N)=O)c21] | [M + H]⁺ 545.3 |

### Ex. 8: Synthesis of N-cyclohexyl-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide

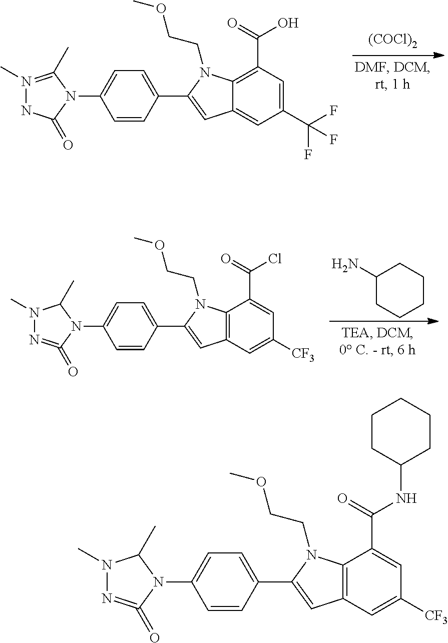

### Step 1

To a stirred solution of 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (60 mg, 0.13 mmol, 1.0 eq) and DMF (1 mg, 0.013 mmol, 0.1 eq) in DCM (2 mL) was added (COCl)2 (48 mg, 0.38 mmol, 3.0 eq) at room temperature, the mixture was stirred for 1 h at room temperature. The resulting mixture was concentrated under vacuum. To the above mixture was added cyclohexylamine (19 mg, 0.19 mmol, 1.5 eq) and TEA (38 mg, 0.38 mmol, 3.0 eq) at 0° C. Then the resulting mixture was stirred for an additional 6 h at room temperature. The resulting mixture was concentrated under vacuum. The residue was purified by silica gel column chromatography (eluting with DCM:MeOH=10:1) to give N-cyclohexyl-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide (11 mg, 15.9%) as a yellow solid. ¹H NMR (400 MHz, CDCl3): δ 7.98 (s, 1H), 7.73-7.63 (m, 2H), 7.48-7.36 (m, 3H), 6.69 (s, 1H), 6.09-5.99 (m, 1H), 4.64-4.54 (m, 2H), 4.08-3.97 (m, 1H), 3.52 (s, 3H), 3.42-3.32 (m, 2H), 3.00 (s, 3H), 2.23 (s, 3H), 2.17-2.06 (m, 2H), 1.84-1.76 (m, 2H), 1.73-1.66 (m, 1H), 1.50-1.42 (m, 1H), 1.37-1.25 (m, 4H). ESI-MS m/z=556.2 [M+H]⁺.

The following examples were prepared using procedures similar to those described in Ex. 6:

| Example | Structure | ESI-MS |
|---|---|---|
| 9 | 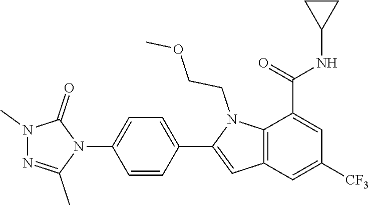 [SMILES: COCCn1c(-c2ccc(-n3c(C)nn(C)c3=O)cc2)cc2cc(C(F)(F)F)cc(C(=O)NC3CC3)c21] | [M + H]⁺ 514.2 |
| 10 | 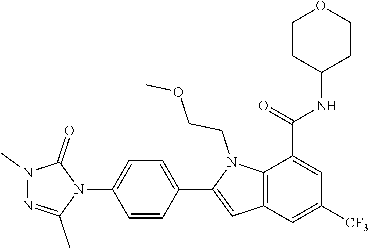 [SMILES: COCCn1c(-c2ccc(-n3c(C)nn(C)c3=O)cc2)cc2cc(C(F)(F)F)cc(C(=O)NC3CCOCC3)c21] | [M + H]⁺ 558.2 |

### Ex. 11: Synthesis of N-((1S,3S)-3-carbamoylcyclohexyl)-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide

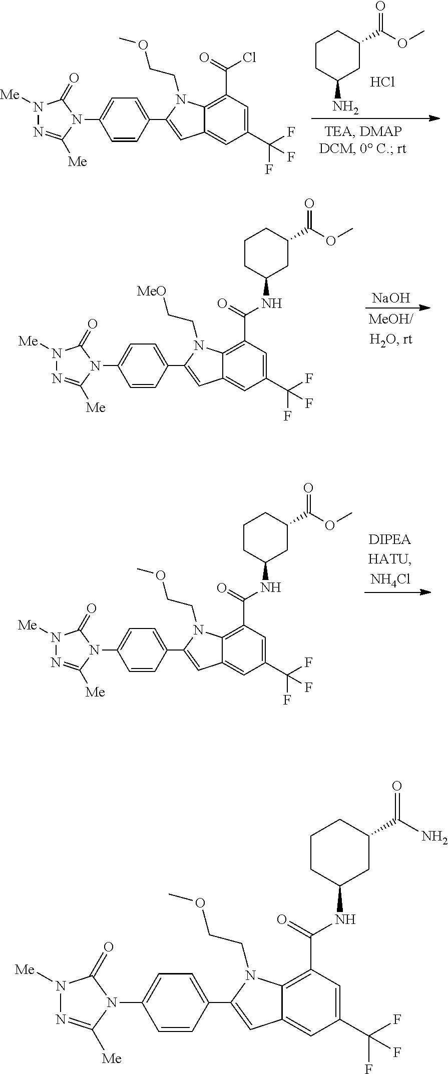

### Step 1

To a solution of 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carbonyl chloride (80 mg, 0.16 mmol, 1.0 eq) in DCM (2 mL) was added TEA (164 mg, 1.62 mmol, 10.0 eq) and methyl (1S,3S)-3-aminocyclohexane-1-carboxylate (63 mg, 0.32 mmol, 2.0 eq) at 0° C. under a nitrogen atmosphere. The reaction mixture was stirred at room temperature for 2 h, then diluted with water (3 mL) and extracted with EtOAc (3 mL×3). The combined organic layers were washed with brine (2 mL), dried over Na₂SO₄ and concentrated. The residue was purified by silica gel column chromatography (DCM/MeOH=97/3) to give the methyl (1S,3S)-3-(2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamido)cyclohexane-1-carboxylate (55 mg, 55%) as a yellow solid. ¹H NMR (400 MHz, CD₃OD): δ 8.04 (s, 1H), 7.79-7.74 (m, 2H), 7.59-7.53 (m, 2H), 7.49 (d, J=1.6 Hz, 1H), 6.82 (s, 1H), 4.57 (t, J=5.6 Hz, 2H), 4.32-4.23 (m, 1H), 3.70 (s, 3H), 3.47 (s, 3H), 3.39-3.35 (m, 2H), 2.97 (s, 3H), 2.21 (s, 3H), 2.18-2.11 (m, 1H), 1.96-1.80 (m, 3H), 1.77-1.54 (m, 5H).

### Step 2

To a solution of methyl (1S,3S)-3-(2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamido)cyclohexane-1-carboxylate (55 mg, 0.09 mmol, 1.0 eq) in 3 mL MeOH/water (v/v=3/2) was added NaOH (18 mg, 0.45 mmol, 5.0 eq), the reaction mixture was stirred at room temperature for 12 h. The mixture was adjusted pH to 2-3 with HCl (1 N). The resulting mixture was extracted with EtOAc (5 mL), the organic phase was concentrated under vacuum to give (1S,3S)-3-(2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamido)cyclohexane-1-carboxylic acid (32 mg, 59%) as a pale yellow solid. ¹H NMR (300 MHz, CD₃OD): δ 8.83 (d, J=7.5 Hz, 1H), 8.02 (s, 1H), 7.77 (d, J=8.4 Hz, 2H), 7.55 (d, J=8.4 Hz, 2H), 7.49 (s, 1H), 6.82 (s, 1H), 4.58 (t, J=5.4 Hz, 2H), 4.40-4.23 (m, 1H), 3.47 (s, 3H), 3.38 (t, J=5.4 Hz, 2H), 2.97 (s, 3H), 2.21 (s, 3H), 2.16-2.09 (m, 1H), 1.95-1.81 (m, 3H), 1.78-1.52 (m, 5H).). ESI-MS m/z=600.1 [M+H]⁺.

### Step 3

To a solution of (1S,3S)-3-(2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamido)cyclohexane-1-carboxylic acid (32 mg, 0.053 mmol, 1.0 eq) in THF (1.5 mL) was added NH₄Cl (11.4 mg, 0.21 mmol, 4.0 eq), DIEA (13.8 mg, 0.11 mmol, 2.0 eq) and HATU (22.1 mg, 0.058 mmol, 1.1 eq). The reaction mixture was stirred at room temperature for 3 h. Upon full conversion, as judged by LCMS analysis of the crude reaction mixture, the reaction mixture was diluted with saturated NaHCO₃ solution (4 mL) and extracted with EtOAc (3 mL×2). The combined organic layers were washed with brine (2 mL), dried over Na₂SO₄ and filtered. Upon concentration of the filtrate, the residue was purified by silica gel column chromatography (DCM/MeOH=93/7) to give N-((1S,3S)-3-carbamoylcyclohexyl)-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide (17 mg, yield 53%) as a white solid. ¹H NMR (400 MHz, DMSO-d6): δ 8.72 (d, J=7.2 Hz, 1H), 8.11 (s, 1H), 7.73 (d, J=8.4 Hz, 2H), 7.59 (d, J=8.4 Hz, 2H), 7.44 (s, 1H), 7.12 (s, 1H), 6.86 (s, 1H), 6.75 (s, 1H), 4.49 (s, 2H), 4.22 (s, 1H), 3.37 (s, 5H), 2.88 (s, 3H), 2.15 (s, 3H), 2.04-1.93 (m, 1H), 1.90-1.80 (m, 1H), 1.79-1.71 (m, 1H), 1.69-1.53 (m, 6H). ESI-MS m/z=599.2 [M+H]⁺.

The following examples were prepared using procedures similar to those described in Ex. 11:

| Example | Structure | ESI-MS |
|---|---|---|
| 12 | 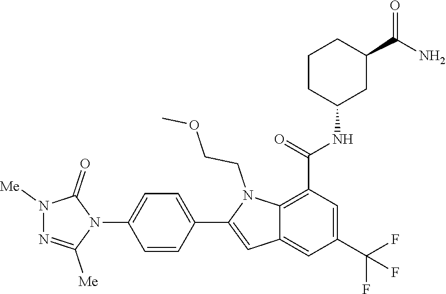 [SMILES: COCCn1c(-c2ccc(-n3c(C)nn(C)c3=O)cc2)cc2cc(C(F)(F)F)cc(C(=O)N[C@@H]3CCC[C@@H](C(N)=O)C3)c21] | [M + H]⁺ 599.1 |
| 13 | 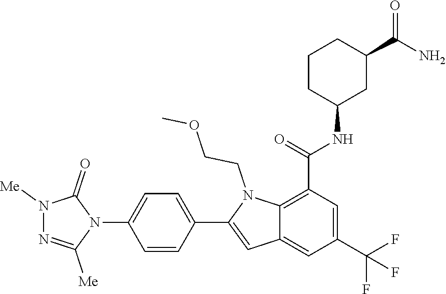 [SMILES: COCCn1c(-c2ccc(-n3c(C)nn(C)c3=O)cc2)cc2cc(C(F)(F)F)cc(C(=O)N[C@H]3CCC[C@@H](C(N)=O)C3)c21] | [M + H]⁺ 599.1 |
| 14 | 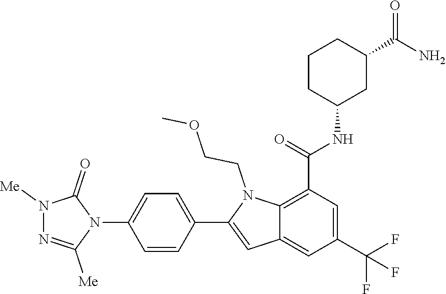 [SMILES: COCCn1c(-c2ccc(-n3c(C)nn(C)c3=O)cc2)cc2cc(C(F)(F)F)cc(C(=O)N[C@@H]3CCC[C@H](C(N)=O)C3)c21] | [M + H]⁺ 599.1 |
| 15 | 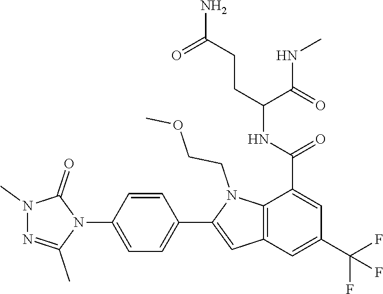 [SMILES: CNC(=O)C(CCC(N)=O)NC(=O)c1cc(C(F)(F)F)cc2cc(-c3ccc(-n4c(C)nn(C)c4=O)cc3)n(CCOC)c12] | [M + H]⁺ 616.2 |

### Ex. 16: Synthesis of N-((2-cyanophenyl)sulfonyl)-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide

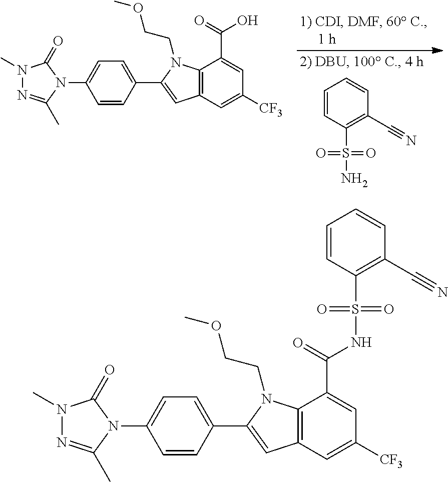

### Step 1

To a solution of 2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxylic acid (100 mg, 0.21 mmol, 1.0 eq) in DMF (2.0 mL) was added 1,1′-Carbonyldiimidazole (137 mg, 0.84 mmol, 4.0 eq). The reaction mixture was stirred at 60° C. for 1 h. The reaction mixture was cooled to room temperature. 1,8-diazabicyclo[5.4.0]undec-7-ene (45 mg, 0.30 mmol, 1.4 eq) and 2-cyanobenzenesulfonamide (54 mg, 0.30 mmol, 1.4 eq) were added. The reaction mixture was then stirred at 100° C. for 4 h. Upon full conversion, as judged by LCMS analysis of the crude reaction mixture, the reaction mixture was cooled to room temperature, and the pH was adjusted to 3-4 using 1 N HCl (aqueous). The aqueous phase was extracted with EtOAc (50 mL), and the organic layer was washed with brine (30 mL×3). Upon concentration the residue was purified by reversed phase chromatography (eluted with ACN/H2O (0.1% TFA in H2O)=55:45) to afford N-((2-cyanophenyl)sulfonyl)-2-(4-(1,3-dimethyl-5-oxo-1,5-dihydro-4H-1,2,4-triazol-4-yl)phenyl)-1-(2-methoxyethyl)-5-(trifluoromethyl)-1H-indole-7-carboxamide (14.6 mg, yield 10.8%) as a white solid. ¹H NMR (400 MHz, DMSO-d6): δ 12.20 (s, 1H), 8.55 (s, 1H), 8.30 (s, 1H), 8.18 (s, 1H), 8.07 (d, J=6.0 Hz, 1H), 7.93 (d, J=3.6 Hz, 2H), 7.75 (d, J=8.4 Hz, 2H), 7.62 (d, J=8.4 Hz, 2H), 6.97 (s, 1H), 4.47 (t, J=5.2 Hz, 2H), 3.41 (t, J=5.2 Hz, 2H), 3.37 (s, 3H), 2.88 (s, 3H), 2.16 (s, 3H). ESI-MS m/z=636.9 [M+H]⁺.

### Ex. 17: Synthesis of N-(3-carbamoyl-2-fluorophenyl)-2-(4-(1,3-dimethyl-5-oxo-1,5-dihydro-4H-1,2,4-triazol-4-yl)-2-methoxyphenyl)-1-(2-methoxyethyl)-5-(trifluoromethyl)-1H-indole-7-carboxamide

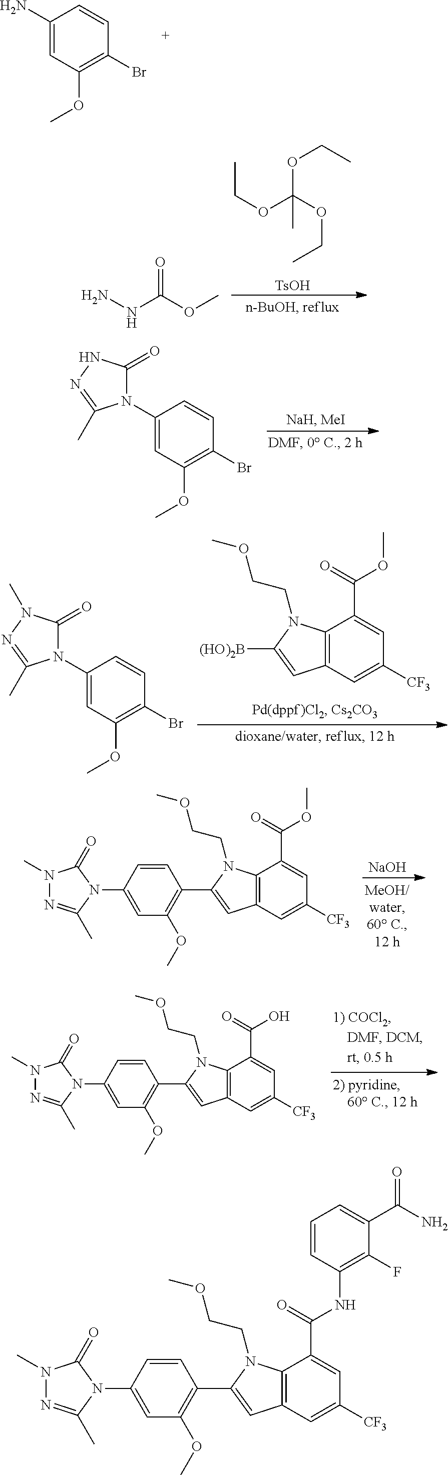

### Step 1

To a solution of 4-bromo-3-methoxyaniline (10.0 g, 49.5 mmol, 1.0 eq) in n-butanol (100 mL) was added methyl hydrazinecarboxylate (5.4 g, 59.4 mmol, 1.2 eq), 1,1,1-triethoxyethane (9.6 g, 59.4 mmol, 1.2 eq) and p-toluenesulfonic acid (0.5 g, 3.0 mmol, 0.06 eq) at room temperature. The reaction mixture was heated at reflux for 12 h, then it was concentrated, and the residue was dissolved in EtOAc (200.0 mL) and washed with brine (200.0 mL×2). The organic layer was concentrated, and the residue was purified by silica gel column chromatography (100% EtOAc) to give 4-(4-bromo-3-methoxyphenyl)-5-methyl-2,4-dihydro-3H-1,2,4-triazol-3-one (9.1 g, yield 65%) as a white solid. ¹H NMR (300 MHz, DMSO-d6): δ 11.63 (s, 1H), 7.71 (d, J=9.0 Hz, 1H), 7.20 (d, J=3.0 Hz, 1H), 7.00-6.90 (m, 1H), 3.87 (s, 3H), 2.08 (s, 3H). ESI-MS m/z=284.0 [M+H]⁺.

### Step 2

To a solution of 4-(4-bromo-3-methoxyphenyl)-5-methyl-2,4-dihydro-3H-1,2,4-triazol-3-one (8.4 g, 29.6 mmol, 1.0 eq) in DMF (85.0 mL) at 0° C. was added NaH (60%, 3.5 g, 88.7 mmol, 3.0 eq) in portions under N2 atmosphere. The resulting mixture was stirred for 1 h, then Mel (12.6 g, 88.7 mmol, 3.0 eq) was added. After 1 h the reaction was quenched with water (100 mL). The aqueous layer was extracted with EtOAc (70 mL×3), and the combined organic layers were washed with brine (100 mL×2) before being concentrated to afford a residue that was purified by silica gel column chromatography (petroleum ether/EtOAc=7/3) to give 4-(4-bromo-3-methoxyphenyl)-2,5-dimethyl-2,4-dihydro-3H-1,2,4-triazol-3-one (6.5 g, yield 73%) as a white solid. ESI-MS m/z=298.3 [M+H]⁺.

### Step 3

To a solution of 4-(4-bromo-3-methoxyphenyl)-2,5-dimethyl-2,4-dihydro-3H-1,2,4-triazol-3-one (250.0 mg, 0.8 mmol, 1.0 eq) and (7-(methoxycarbonyl)-1-(2-methoxyethyl)-5-(trifluoromethyl)-1H-indol-2-yl)boronic acid (579.0 mg, 1.7 mmol, 2.0 eq) in dioxane (5.0 mL)/water (1.0 mL) were added Cs₂CO₃ (547.0 mg, 1.7 mmol, 2.0 eq) and Pd(dppf)Cl₂ (61.0 mg, 0.08 mmol, 0.1 eq) under a nitrogen atmosphere. The mixture was heated to reflux and stirred overnight. Upon cooling to room temperature, the reaction was poured into water (5.0 mL) and extracted with EtOAc (5.0 mL×3). The combined organic layers were concentrated, and the residue was purified by silica gel column chromatography (100% EtOAc) to give methyl 2-(4-(1,3-dimethyl-5-oxo-1,5-dihydro-4H-1,2,4-triazol-4-yl)-2-methoxyphenyl)-1-(2-methoxyethyl)-5-(trifluoromethyl)-1H-indole-7-carboxylate (310.0 mg, yield 71%) as a yellow solid. ¹H NMR (400 MHz, DMSO-d6): δ 8.22 (s, 1H), 7.78 (d, J=1.6 Hz, 1H), 7.51 (d, J=8.0 Hz, 1H), 7.30 (d, J=2.0 Hz, 1H), 7.17 (dd, J=8.0, 2.0 Hz, 1H), 6.81 (s, 1H), 4.43-4.29 (m, 2H), 3.94 (s, 3H), 3.82 (s, 3H), 3.37 (s, 3H), 3.25 (t, J=5.2 Hz, 2H), 2.90 (s, 3H), 2.17 (s, 3H). ESI-MS m/z=519.2 [M+H]⁺.

### Step 4

To a solution of methyl 2-(4-(1,3-dimethyl-5-oxo-1,5-dihydro-4H-1,2,4-triazol-4-yl)-2-methoxyphenyl)-1-(2-methoxyethyl)-5-(trifluoromethyl)-1H-indole-7-carboxylate (310.0 mg, 0.6 mmol, 1.0 eq) in MeOH (4.0 mL)/water (1.0 mL) was added NaOH (71.7 mg, 3.6 mmol, 6.0 eq). The mixture was stirred at 60° C. for 12 h, then it was cooled to room temperature and acidified with 1 N HCl. The solid was filtered, washed with water (2.0 mL), and dried to give 2-(4-(1,3-dimethyl-5-oxo-1,5-dihydro-4H-1,2,4-triazol-4-yl)-2-methoxyphenyl)-1-(2-methoxyethyl)-5-(trifluoromethyl)-1H-indole-7-carboxylic acid (250.0 mg, yield 83%) as a brown solid. 1H NMR (400 MHz, DMSO-d6): δ 8.19 (s, 1H), 7.77 (d, J=1.6 Hz, 1H), 7.51 (d, J=8.0 Hz, 1H), 7.29 (d, J=1.6 Hz, 1H), 7.19-7.12 (m, 1H), 6.79 (s, 1H), 4.43 (s, 2H), 3.82 (s, 3H), 3.37 (s, 3H), 3.30-3.26 (m, 2H), 2.89 (s, 3H), 2.17 (s, 3H). ESI-MS m/z=505.1[M+H]⁺.

### Step 5

To a solution of 2-(4-(1,3-dimethyl-5-oxo-1,5-dihydro-4H-1,2,4-triazol-4-yl)-2-methoxyphenyl)-1-(2-methoxyethyl)-5-(trifluoromethyl)-1H-indole-7-carboxylic acid (120.0 mg, 0.2 mmol, 1.0 eq) in DCM (3.0 mL) was added DMF (1.0 mg, 0.02 mmol, 0.1 eq), then oxalyl chloride (90.0 mg, 0.6 mmol, 3.0 eq) was added under N2 atmosphere. The mixture was stirred at room temperature for 0.5 h, then it was concentrated in vacuum. The residue dissolved by pyridine (3.0 mL), then 3-amino-2-fluorobenzamide (73.2 mg, 0.4 mmol, 2.0 eq) was added. The mixture was stirred at 60° C. under N₂ atmosphere, for 12 h. The mixture was concentrated and was purified by flash (DCM/MeOH=10/1) to give N-(3-carbamoyl-2-fluorophenyl)-2-(4-(1,3-dimethyl-5-oxo-1,5-dihydro-4H-1,2,4-triazol-4-yl)-2-methoxyphenyl)-1-(2-methoxyethyl)-5-(trifluoromethyl)-1H-indole-7-carboxamide (16.1 mg, yield 11%) as a white solid.

1H NMR (400 MHz, DMSO-d6): δ 10.77 (s, 1H), 8.18 (s, 1H), 7.87-7.78 (m, 2H), 7.68 (s, 2H), 7.54-7.46 (m, 2H), 7.35-7.26 (m, 2H), 7.19-7.13 (m, 1H), 6.79 (s, 1H), 4.36-4.22 (m, 2H), 3.80 (s, 3H), 3.38-3.36 (m, 2H), 3.35 (s, 3H), 2.86 (s, 3H), 2.16 (s, 3H). ESI-MS m/z=641.1 [M+H]⁺.

### Ex. 18 Synthesis of N-(4-(3,3-dimethylureido)benzyl)-1-ethyl-2-(4-(2-oxopyridin-1(2H)-yl)phenyl)-5-(trifluoromethyl)-1H-pyrrolo[2,3-c]pyridine-7-carboxamide

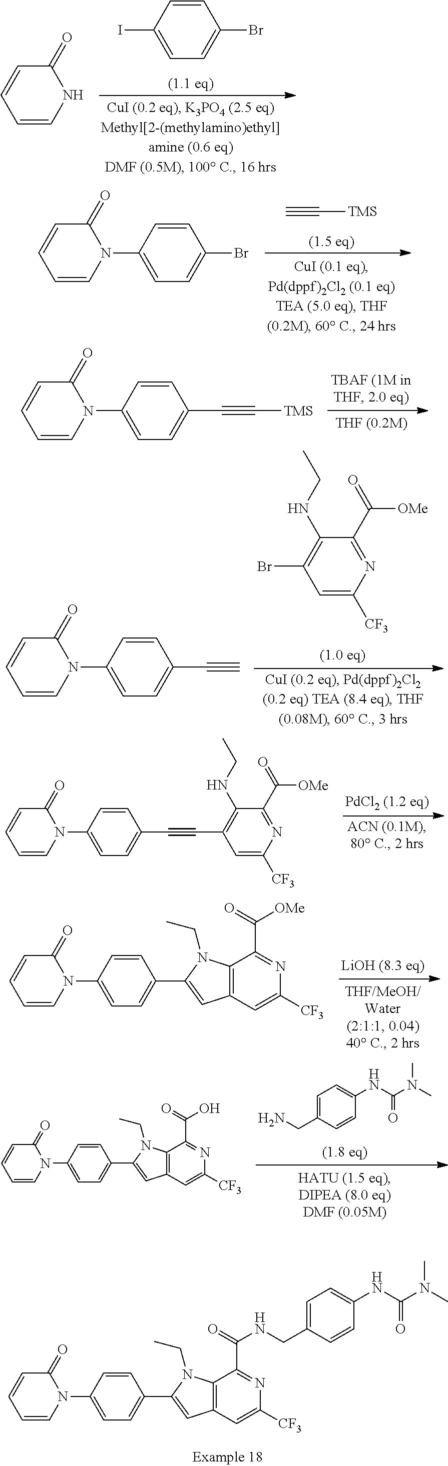

### Step 1:

To a 20 mL vial were added 1-bromo-4-iodobenzene (1.90 g, 1.1 Eq, 6.71 mmol), CuI (232 mg, 0.2 Eq, 1.22 mmol), tripotassium phosphate (3.24 g, 2.5 Eq, 15.2 mmol) and pyridin-2(1H)-one (580 mg, 1 Eq, 6.10 mmol). The vial was evacuated and refilled with N2 3 times. DMF (12.2 mL) was added, followed by Methyl[2-(methylamino)ethyl]amine (323 mg, 393 μL, 0.6 Eq, 3.66 mmol). The mixture was heated at 100° C. over 16 hours. After completion, the reaction mixture was concentrated and subjected to Combi-Flash (Hex:Ace 100:0 to 0:100) to afford 1-(4-bromophenyl)pyridin-2(1H)-one (460 mg, 1.84 mmol, 30.2%)

### Step 2:

To the 20 mL vial were added 1,1′-Bis(diphenylphosphino)ferrocene-palladium(II) dichloride (135 mg, 0.1 Eq, 184 μmol), CuI (35.0 mg, 0.1 Eq, 184 μmol) and 1-(4-bromophenyl)pyridin-2(1H)-one (460 mg, 1 Eq, 1.84 mmol). The vial was then sealed and purged with N2 3 times before ethynyltrimethylsilane (271 mg, 382 μL, 1.5 Eq, 2.76 mmol), TEA (931 mg, 1.28 mL, 5 Eq, 9.20 mmol) and THF (9.20 mL) were added. The reaction mixture was heated to 60° C. for 24 hours. After completion, the mixture was subjected to Combi-Flash (Hex:Ace 100:0 to 0:100) to afford 1-(4-((trimethylsilyl)ethynyl)phenyl)pyridin-2(1H)-one (400 mg, 1.50 mmol, 81.3%).

### Step 3:

To the solution of 1-(4-((trimethylsilyl)ethynyl)phenyl)pyridin-2(1H)-one (400 mg, 1 Eq, 1.50 mmol) was added tetrabutylammonium fluoride (782 mg, 2.99 mL, 1 molar, 2 Eq, 2.99 mmol). The reaction mixture was stirred at 25° C. for 2 hours. After completion, the reaction mixture was directly subjected to Combi-Flash (Hex:Ace 100:0 to 0:100) to afford 1-(4-ethynylphenyl)pyridin-2(1H)-one (239 mg, 1.22 mmol, 81.8%)

### Step 4:

To the 20 mL vial were added methyl 4-bromo-3-(ethylamino)-6-(trifluoromethyl)picolinate (25 mg, 1 Eq, 76 μmol), 1-(4-ethynylphenyl)pyridin-2(1H)-one (22 mg, 1.5 Eq, 0.11 mmol), CuI (2.9 mg, 0.2 Eq, 15 μmol) and 1,1′-Bis(diphenylphosphino)ferrocene-palladium(II) dichloride (11 mg, 0.2 Eq, 15 μmol). The vial was then sealed and purged with N2 3 times before THF (1 mL) and TEA (65 mg, 90 μL, 8.4 Eq, 0.65 mmol) were added. The mixture was heated to 60° C. for 3 hour. After completion, the reaction mixture was directly subjected to CombiFlash (Hex:Ace 100:0 to 0:100) to afford methyl 3-(ethylamino)-4-((4-(2-oxopyridin-1(2H)-yl)phenyl)ethynyl)-6-(trifluoromethyl)picolinate (17 mg, 39 μmol, 50%) as pale yellow solid.

### Step 5:

To the solution of methyl 3-(ethylamino)-4-((4-(2-oxopyridin-1(2H)-yl)phenyl)ethynyl)-6-(trifluoromethyl)picolinate (17 mg, 1 Eq, 39 μmol) in Acetonitrile (0.39 mL) was added palladium(II) chloride (8.2 mg, 1.2 Eq, 46 μmol). The reaction mixture was then heated to 80° C. for 2 hours. After completion, the reaction mixture was dried under vacuum and used directly without further purification in the next step.

### Step 6:

To the solution of methyl 1-ethyl-2-(4-(2-oxopyridin-1(2H)-yl)phenyl)-5-(trifluoromethyl)-1H-pyrrolo[2,3-c]pyridine-7-carboxylate (17 mg, 1.0 Eq, 39 μmol) in THF (0.50 mL), Methanol (0.25 mL) and Water (0.25 mL) was added lithium hydroxide (7.7 mg, 8.3 Eq, 0.32 mmol). The reaction was heated to 40° C. for 2 hours. After completion, the reaction mixture was concentrated and used in the next step as crude.

### Step 7:

To the solution of 1-ethyl-2-(4-(2-oxopyridin-1(2H)-yl)phenyl)-5-(trifluoromethyl)-1H-pyrrolo[2,3-c]pyridine-7-carboxylic acid (17 mg, 1 Eq, 40 μmol) in DMF (0.80 mL) were added 3-(4-(aminomethyl)phenyl)-1,1-dimethylurea (14 mg, 1.8 Eq, 72 μmol), DIPEA (41 mg, 55 μL, 8 Eq, 0.32 mmol) and HATU (23 mg, 1.5 Eq, 60 μmol). After completion, the reaction mixture was dried under vacuum, and subjected to Combi-Flash (Hex:Ace 100:0 to 0:100) to afford N-(4-(3,3-dimethylureido)benzyl)-1-ethyl-2-(4-(2-oxopyridin-1(2H)-yl)phenyl)-5-(trifluoromethyl)-1H-pyrrolo[2,3-c]pyridine-7-carboxamide (5 mg, 8 μmol, 20%). ESI-MS m/z=603.952 [M+H]⁺.

The following examples were prepared using procedures similar to those described in Ex. 18:

| Ex- | | |
|---|---|---|
| ample | Structure | ESI-MS |
| 19 | 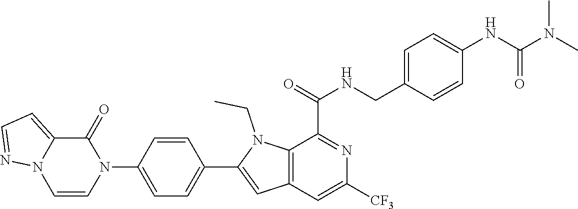 [SMILES: CCn1c(-c2ccc(-n3ccn4nccc4c3=O)cc2)cc2cc(C(F)(F)F)nc(C(=O)NCc3ccc(NC(=O)N(C)C)cc3)c21] | [M + H]⁺ 643.84 |
| 20 | 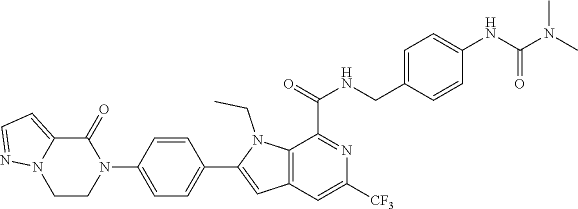 [SMILES: CCn1c(-c2ccc(N3CCn4nccc4C3=O)cc2)cc2cc(C(F)(F)F)nc(C(=O)NCc3ccc(NC(=O)N(C)C)cc3)c21] | [M + H]⁺ 645.81 |
| 21 | 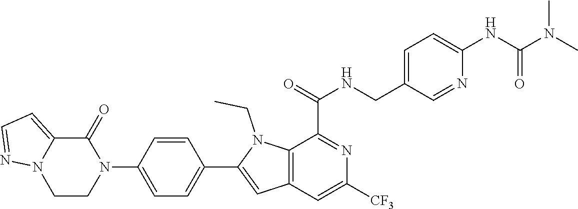 [SMILES: CCn1c(-c2ccc(N3CCn4nccc4C3=O)cc2)cc2cc(C(F)(F)F)nc(C(=O)NCc3ccc(NC(=O)N(C)C)nc3)c21] | [M + H]⁺ 646.77 |
| 22 | 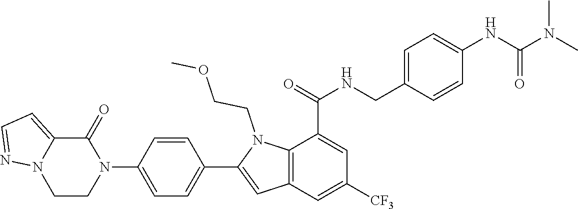 [SMILES: COCCn1c(-c2ccc(N3CCn4nccc4C3=O)cc2)cc2cc(C(F)(F)F)cc(C(=O)NCc3ccc(NC(=O)N(C)C)cc3)c21] | [M + H]⁺ 674.95 |
| 23 | 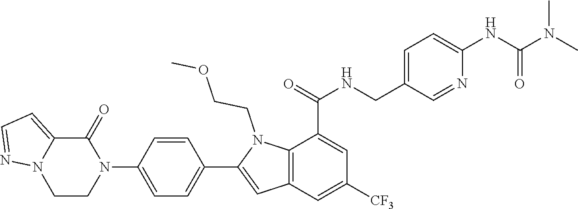 [SMILES: COCCn1c(-c2ccc(N3CCn4nccc4C3=O)cc2)cc2cc(C(F)(F)F)cc(C(=O)NCc3ccc(NC(=O)N(C)C)nc3)c21] | [M + H]⁺ 676.00 |
| 24 | 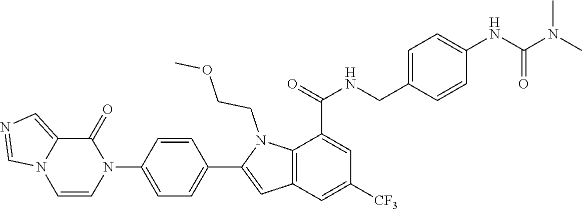 [SMILES: COCCn1c(-c2ccc(-n3ccn4cncc4c3=O)cc2)cc2cc(C(F)(F)F)cc(C(=O)NCc3ccc(NC(=O)N(C)C)cc3)c21] | [M + H]⁺ 672.88 |
| 25 | 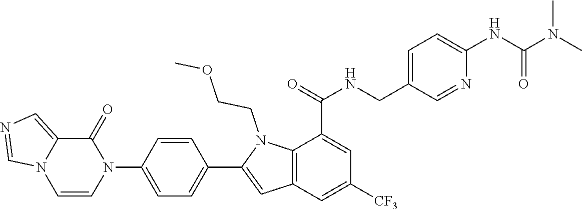 [SMILES: COCCn1c(-c2ccc(-n3ccn4cncc4c3=O)cc2)cc2cc(C(F)(F)F)cc(C(=O)NCc3ccc(NC(=O)N(C)C)nc3)c21] | [M + H]⁺ 673.70 |
| 26 | 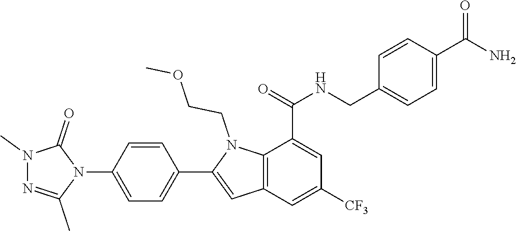 [SMILES: COCCn1c(-c2ccc(-n3c(C)nn(C)c3=O)cc2)cc2cc(C(F)(F)F)cc(C(=O)NCc3ccc(C(N)=O)cc3)c21] | [M + H]⁺ 607.8 |
| 27 | 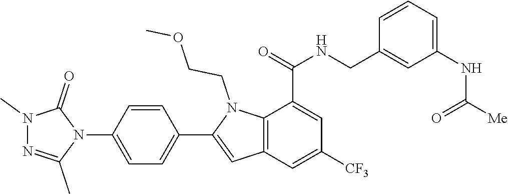 [SMILES: COCCn1c(-c2ccc(-n3c(C)nn(C)c3=O)cc2)cc2cc(C(F)(F)F)cc(C(=O)NCc3cccc(NC(C)=O)c3)c21] | [M + H]⁺ 621.8 |
| 28 | 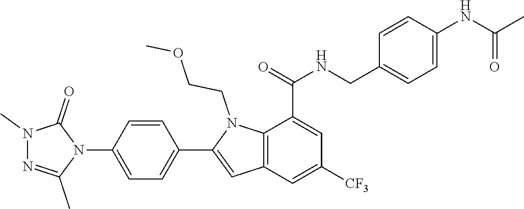 [SMILES: COCCn1c(-c2ccc(-n3c(C)nn(C)c3=O)cc2)cc2cc(C(F)(F)F)cc(C(=O)NCc3ccc(NC(C)=O)cc3)c21] | [M + H]⁺ 621.8 |
| 29 | 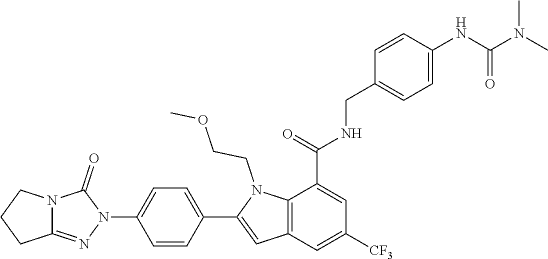 [SMILES: COCCn1c(-c2ccc(-n3nc4n(c3=O)CCC4)cc2)cc2cc(C(F)(F)F)cc(C(=O)NCc3ccc(NC(=O)N(C)C)cc3)c21] | [M + H]⁺ 662.81 |
| 30 | 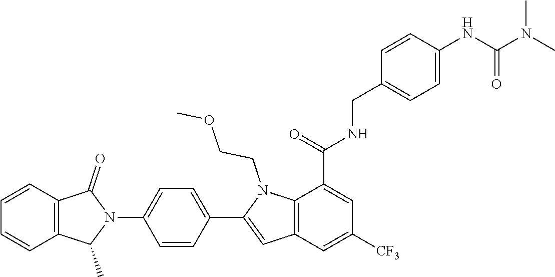 [SMILES: COCCn1c(-c2ccc(N3C(=O)c4ccccc4[C@H]3C)cc2)cc2cc(C(F)(F)F)cc(C(=O)NCc3ccc(NC(=O)N(C)C)cc3)c21] | [M + H]⁺ 684.83 |
| 31 | 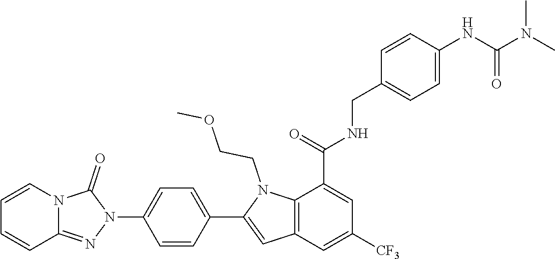 [SMILES: COCCn1c(-c2ccc(-n3nc4ccccn4c3=O)cc2)cc2cc(C(F)(F)F)cc(C(=O)NCc3ccc(NC(=O)N(C)C)cc3)c21] | [M + H]⁺ 672.73 |
| 32 | 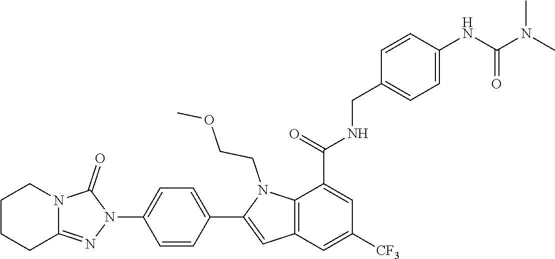 [SMILES: COCCn1c(-c2ccc(-n3nc4n(c3=O)CCCC4)cc2)cc2cc(C(F)(F)F)cc(C(=O)NCc3ccc(NC(=O)N(C)C)cc3)c21] | [M + H]⁺ 676.78 |
| 33 | 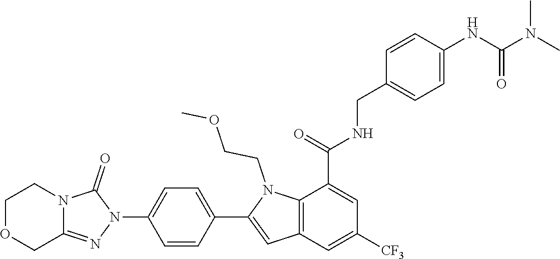 [SMILES: COCCn1c(-c2ccc(-n3nc4n(c3=O)CCOC4)cc2)cc2cc(C(F)(F)F)cc(C(=O)NCc3ccc(NC(=O)N(C)C)cc3)c21] | [M + H]⁺ 678.70 |
| 34 | 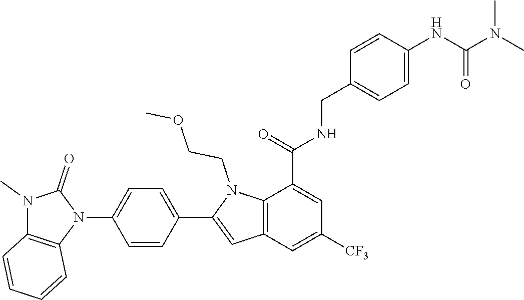 | [M + H]⁺ 685.90 |
| 35 | 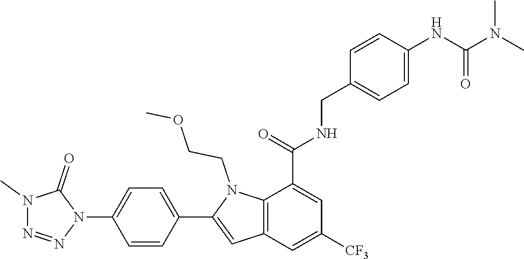 | [M + H]⁺ 637.70 |
| 36 | 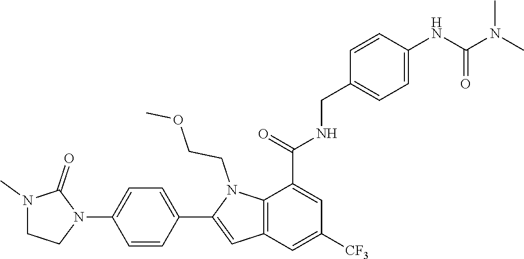 | [M + H]⁺ 637.66 |
| 37 | 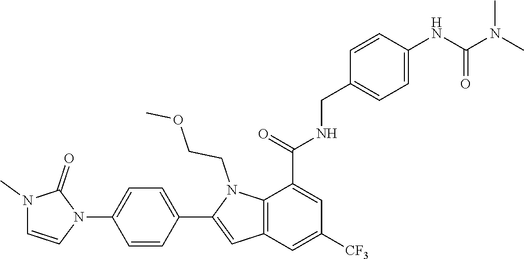 | [M + H]⁺ 635.73 |
| 38 | 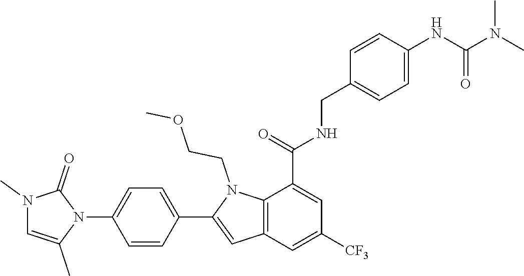 | [M + H]⁺ 649.82 |
| 39 | 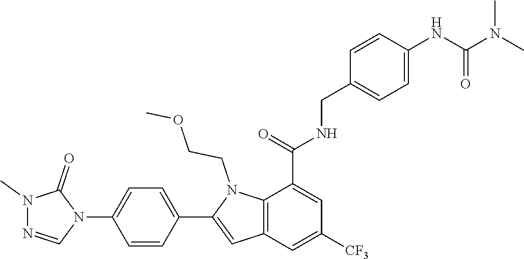 | [M + H]⁺ 636.67 |
| 40 | 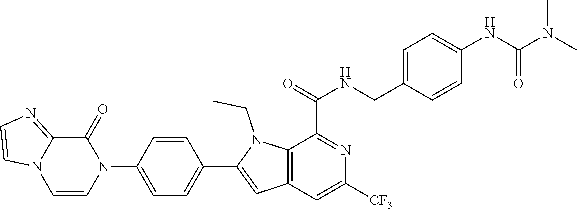 [SMILES: CCn1c(-c2ccc(-n3ccn4ccnc4c3=O)cc2)cc2cc(C(F)(F)F)nc(C(=O)NCc3ccc(NC(=O)N(C)C)cc3)c21] | [M + H]⁺ 643.747 |
| 41 | 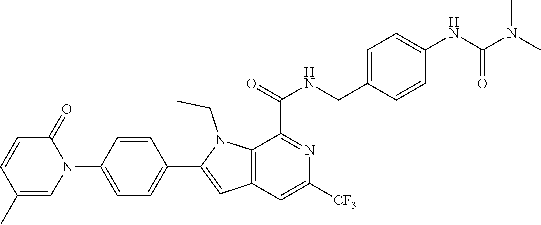 [SMILES: CCn1c(-c2ccc(-n3cc(C)ccc3=O)cc2)cc2cc(C(F)(F)F)nc(C(=O)NCc3ccc(NC(=O)N(C)C)cc3)c21] | [M + H]⁺ 617.873 |
| 42 | 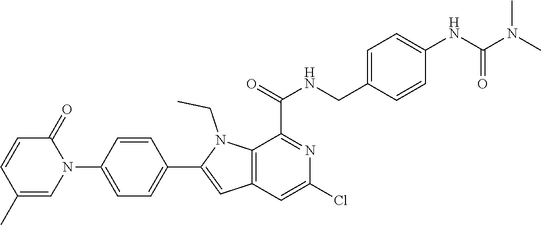 [SMILES: CCn1c(-c2ccc(-n3cc(C)ccc3=O)cc2)cc2cc(Cl)nc(C(=O)NCc3ccc(NC(=O)N(C)C)cc3)c21] | [M + H]⁺ 583.839, 585.711 |
| 43 | 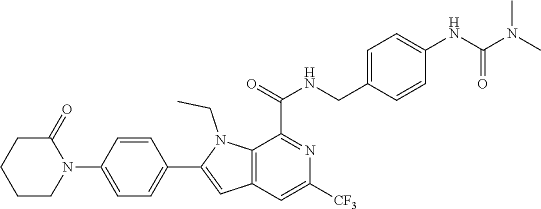 [SMILES: CCn1c(-c2ccc(N3CCCCC3=O)cc2)cc2cc(C(F)(F)F)nc(C(=O)NCc3ccc(NC(=O)N(C)C)cc3)c21] | [M + H]⁺ 607.937 |
| 44 | 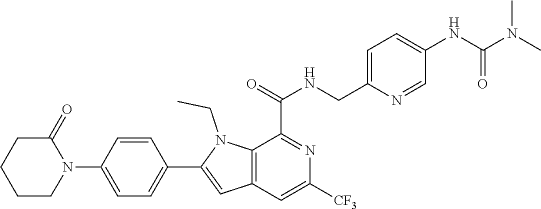 [SMILES: CCn1c(-c2ccc(N3CCCCC3=O)cc2)cc2cc(C(F)(F)F)nc(C(=O)NCc3ccc(NC(=O)N(C)C)cn3)c21] | [M + H]⁺ 608.705 |
| 45 | 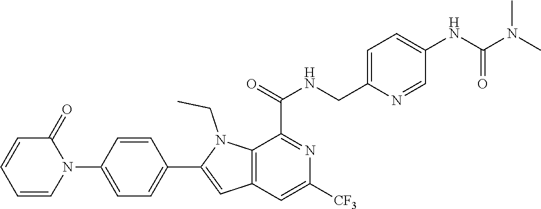 [SMILES: CCn1c(-c2ccc(-n3ccccc3=O)cc2)cc2cc(C(F)(F)F)nc(C(=O)NCc3ccc(NC(=O)N(C)C)cn3)c21] | [M + H]⁺ 604.913 |
| 46 | 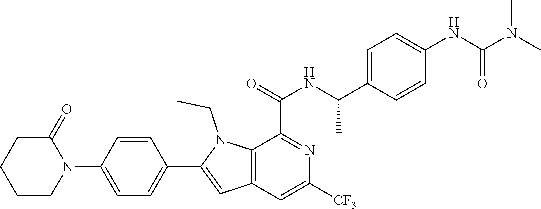 [SMILES: CCn1c(-c2ccc(N3CCCCC3=O)cc2)cc2cc(C(F)(F)F)nc(C(=O)N[C@@H](C)c3ccc(NC(=O)N(C)C)cc3)c21] | [M + H]⁺ 621.906 |
| 47 | 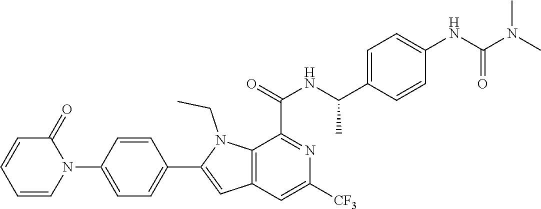 [SMILES: CCn1c(-c2ccc(-n3ccccc3=O)cc2)cc2cc(C(F)(F)F)nc(C(=O)N[C@@H](C)c3ccc(NC(=O)N(C)C)cc3)c21] | [M + H]⁺ 617.681 |
| 48 | 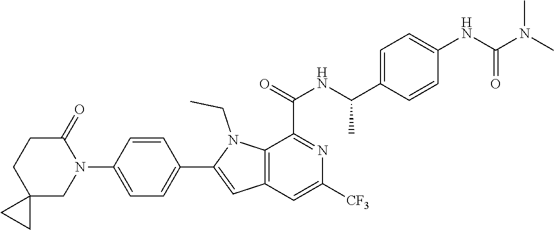 [SMILES: CCn1c(-c2ccc(N3CC4(CCC3=O)CC4)cc2)cc2cc(C(F)(F)F)nc(C(=O)N[C@@H](C)c3ccc(NC(=O)N(C)C)cc3)c21] | [M + H]⁺ 647.827 |
| 49 | 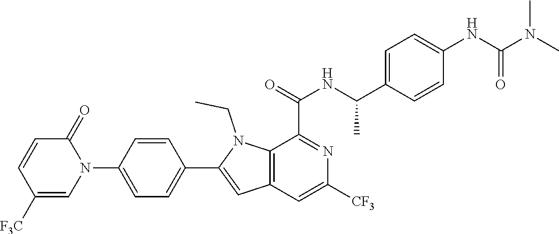 [SMILES: CCn1c(-c2ccc(-n3cc(C(F)(F)F)ccc3=O)cc2)cc2cc(C(F)(F)F)nc(C(=O)N[C@@H](C)c3ccc(NC(=O)N(C)C)cc3)c21] | [M + H]⁺ 685.989 |
| 50 | 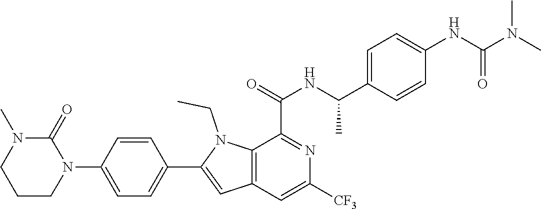 [SMILES: CCn1c(-c2ccc(N3CCCN(C)C3=O)cc2)cc2cc(C(F)(F)F)nc(C(=O)N[C@@H](C)c3ccc(NC(=O)N(C)C)cc3)c21] | [M + H]⁺ 636.979 |
| 51 | 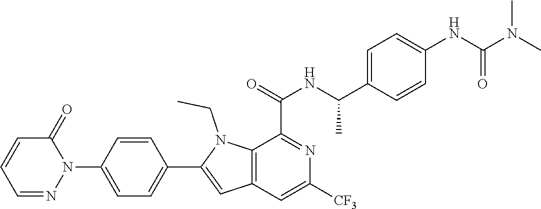 [SMILES: CCn1c(-c2ccc(-n3ncccc3=O)cc2)cc2cc(C(F)(F)F)nc(C(=O)N[C@@H](C)c3ccc(NC(=O)N(C)C)cc3)c21] | [M + H]⁺ 618.785 |
| 52 | 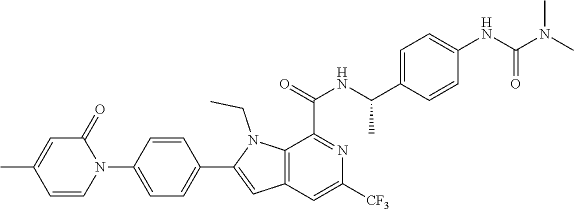 | [M + H]⁺ 631.746 |
| 53 | 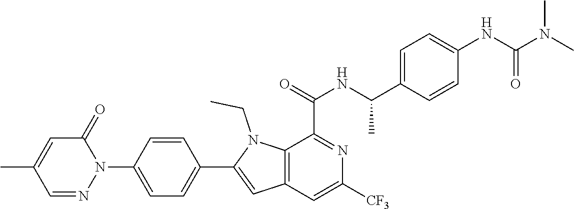 | [M + H]⁺ 632.562 |
| 54 | 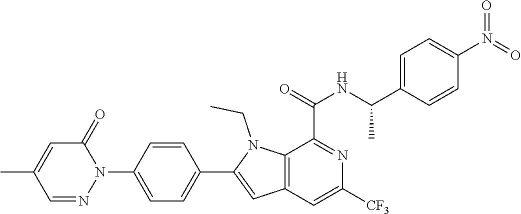 [SMILES: CCn1c(-c2ccc(-n3ncc(C)cc3=O)cc2)cc2cc(C(F)(F)F)nc(C(=O)N[C@@H](C)c3ccc([N+](=O)[O-])cc3)c21] | [M + H]⁺ 591.856 |
| 55 | 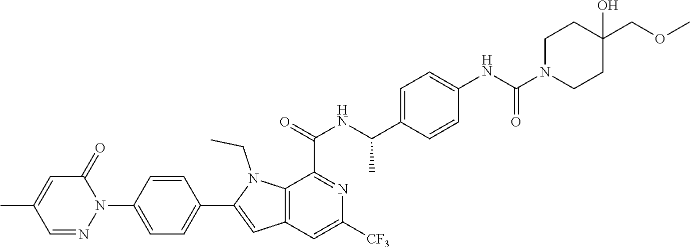 | [M + H]⁺ 732.694 |

The following compounds are prepared in using procedures similar to those described above:

| Example | Structure |
|---|---|
| 1a | 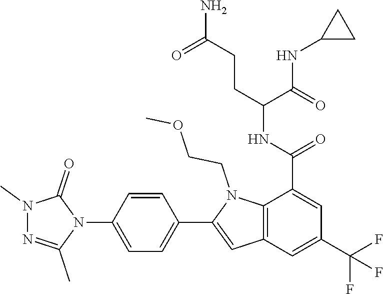 [SMILES: COCCn1c(-c2ccc(-n3c(C)nn(C)c3=O)cc2)cc2cc(C(F)(F)F)cc(C(=O)NC(CCC(N)=O)C(=O)NC3CC3)c21] |
| 2a | 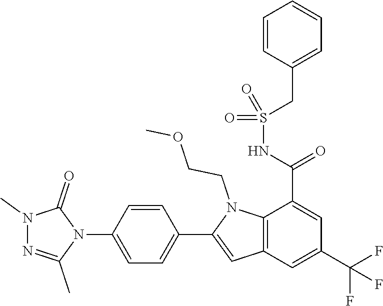 [SMILES: COCCn1c(-c2ccc(-n3c(C)nn(C)c3=O)cc2)cc2cc(C(F)(F)F)cc(C(=O)NS(=O)(=O)Cc3ccccc3)c21] |
| 3a | 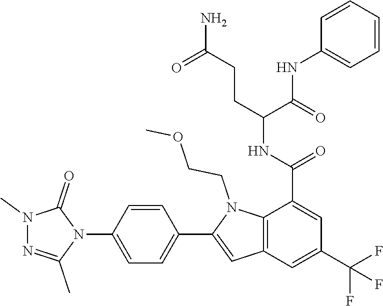 [SMILES: COCCn1c(-c2ccc(-n3c(C)nn(C)c3=O)cc2)cc2cc(C(F)(F)F)cc(C(=O)NC(CCC(N)=O)C(=O)Nc3ccccc3)c21] |
| 4a | 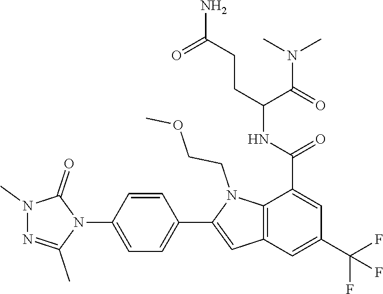 [SMILES: COCCn1c(-c2ccc(-n3c(C)nn(C)c3=O)cc2)cc2cc(C(F)(F)F)cc(C(=O)NC(CCC(N)=O)C(=O)N(C)C)c21] |
| 5a | 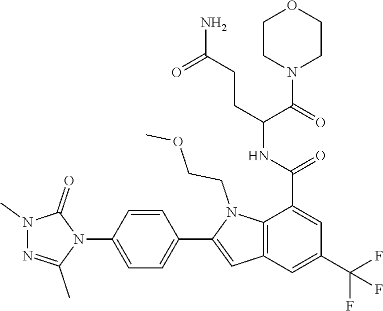 [SMILES: COCCn1c(-c2ccc(-n3c(C)nn(C)c3=O)cc2)cc2cc(C(F)(F)F)cc(C(=O)NC(CCC(N)=O)C(=O)N3CCOCC3)c21] |
| 6a | 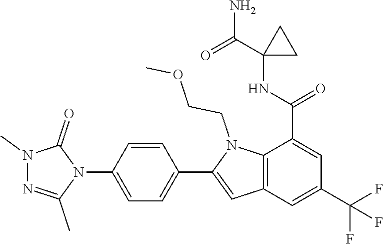 [SMILES: COCCn1c(-c2ccc(-n3c(C)nn(C)c3=O)cc2)cc2cc(C(F)(F)F)cc(C(=O)NC3(C(N)=O)CC3)c21] |
| 7a | 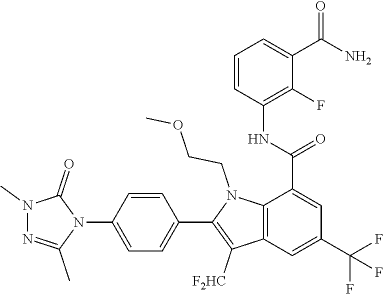 [SMILES: COCCn1c(-c2ccc(-n3c(C)nn(C)c3=O)cc2)c(C(F)F)c2cc(C(F)(F)F)cc(C(=O)Nc3cccc(C(N)=O)c3F)c21] |
| 8a | 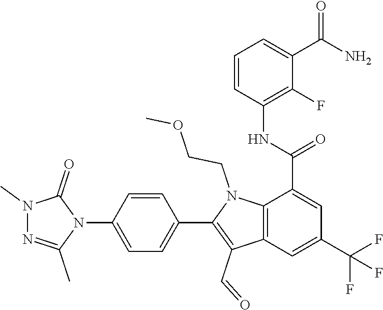 [SMILES: COCCn1c(-c2ccc(-n3c(C)nn(C)c3=O)cc2)c(C=O)c2cc(C(F)(F)F)cc(C(=O)Nc3cccc(C(N)=O)c3F)c21] |
| 9a | 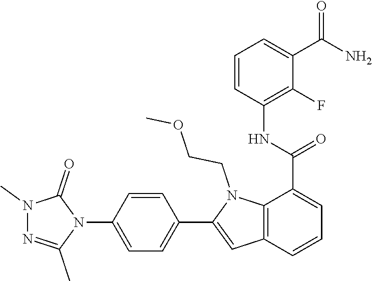 [SMILES: COCCn1c(-c2ccc(-n3c(C)nn(C)c3=O)cc2)cc2cccc(C(=O)Nc3cccc(C(N)=O)c3F)c21] |
| 10a | 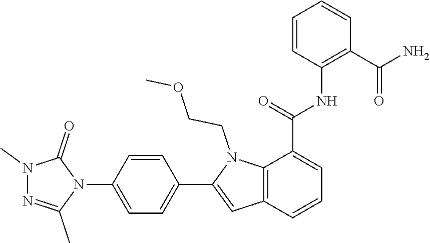 [SMILES: COCCn1c(-c2ccc(-n3c(C)nn(C)c3=O)cc2)cc2cccc(C(=O)Nc3ccccc3C(N)=O)c21] |
| 11a | 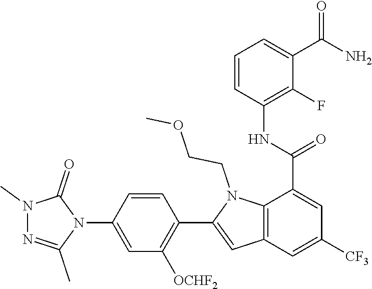 [SMILES: COCCn1c(-c2ccc(-n3c(C)nn(C)c3=O)cc2OC(F)F)cc2cc(C(F)(F)F)cc(C(=O)Nc3cccc(C(N)=O)c3F)c21] |
| 12a | 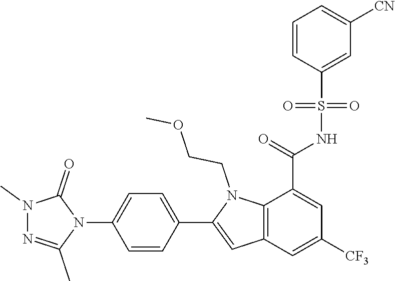 [SMILES: COCCn1c(-c2ccc(-n3c(C)nn(C)c3=O)cc2)cc2cc(C(F)(F)F)cc(C(=O)NS(=O)(=O)c3cccc(C#N)c3)c21] |
| 13a | 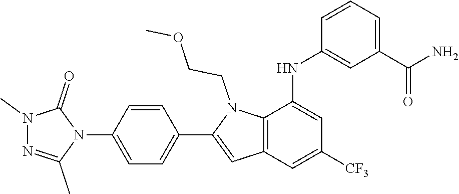 [SMILES: COCCn1c(-c2ccc(-n3c(C)nn(C)c3=O)cc2)cc2cc(C(F)(F)F)cc(Nc3cccc(C(N)=O)c3)c21] |
| 14a | 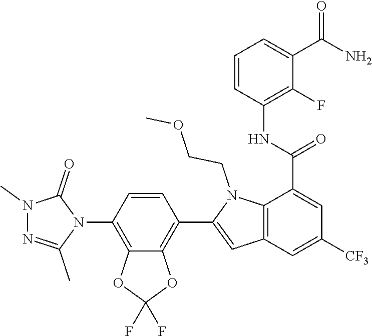 [SMILES: COCCn1c(-c2ccc(-n3c(C)nn(C)c3=O)c3c2OC(F)(F)O3)cc2cc(C(F)(F)F)cc(C(=O)Nc3cccc(C(N)=O)c3F)c21] |

### Biological Activity

### STAT6 FP Activity

For a competition-based fluorescence polarization (FP) assay, the ability of the test compounds to displace the fluorophore-labeled STAT6 peptide substrate at the SH2 binding domain of STAT6 was analyzed. Test compounds were dispersed into a 384-well low volume black ProxiPlate microplate from a DMSO solution using an ECHO 650 acoustic Test compounds were dispersed into a 384-well low volume while ProxiPlate microplate from a DMSO solution using an ECHO 650 acoustic dispenser. Recombinant human STAT6 core domain protein (STAT6 H122-T658) at 125 nM in FP assay buffer (10 mM HEPES pH 7.5, 50 mM NaCl, 1 mM EDTA, 0.05% Tween-20, 2 mM DTT) was added to the test or high control wells. FP assay buffer was added to the low control wells. The plate was incubated at RT for 30 min. Next, 2 nM of buffered STAT6 peptide probe [5-FAM-G(pY)VPWQDLI-NH2] solution was added to all wells. The plate was then incubated at RT for another 30 min. FP (milliPolarization—mP value) was measured at RT in an Envision plate reader equipped with 485 nm excitation and 520 nm emission filters, operating in endpoint mode.

FP signal (mP values) from high and low control wells were used to calculate normalized STAT6 binding activity at various concentrations of test compounds. The normalized activities were fitted to inhibitor-versus-normalized response fit in GraphPad Prism 7 to determine half-maximal inhibitory concentration (IC50). IC₅₀ ranges are reported as follows: A<1 μM; B 1-10 μM; C 10-50 μM; D>50 μM.

TABLE 1
STAT6 Binding Activity

| Ex. # | STAT6 FP IC₅₀ | Ex. # | STAT6 FP IC₅₀ |
|---|---|---|---|
| 1 | A | 2 | B |
| 3 | D | 4 | A |
| 5 | A | 6 | D |
| 7 | C | 8 | D |
| 9 | C | 10 | D |
| 11 | D | 12 | D |
| 13 | B | 14 | D |
| 15 | D | 16 | A |
| 17 | A | 18 | ? |
| 19 | B | 26 | B |
| 27 | D | 28 | B |
| 40 | C | 41 | A |
| 42 | B | 43 | B |
| 44 | C | 45 | B |
| 46 | D | 47 | B |
| 48 | B | 49 | D |

### STAT6 HEK-BLUE IL-4 & IL-13 Assay

HEK-BLUE IL-4 & IL-13 cells stably expressing STAT6 and a STAT6-inducible secreted embryonic alkaline phosphatase reporter were maintained in growth media consisting of Dulbecco's Modified Eagle Medium plus GlutaMAX supplemented with 10% heat-inactivated fetal bovine serum, 100 U/mL penicillin, 100 μg/mL streptomycin, 100 μg/mL Normocin, 10 μg/mL blasticidin and 100 μg/mL Zeocin. Test media consisted of growth media with the exclusion of Normocin, blasticidin and Zeocin. All cell maintenance and incubations were performed at 37° C., 5% CO₂ in a humidified incubator.

In a 384-well plate, compounds, solubilized in DMSO, were dispensed using an ECHO 650 acoustic liquid handler. HEK-BLUE IL-4 & IL-13 cells were resuspended in test media at a density of 12,000 cells per well and incubated with dispensed compounds for three hours. IL-4 was solubilized in 0.1% human serum albumin (HSA) in PBS and cells were stimulated with a pre-determined IL-4 EC75. Plates were incubated overnight. The following day, QUANTI-Blue was added per manufacturer's instructions. One hour post incubation, absorbance was measured at a wavelength of 620 nm on an Envision plate reader. Absorbance values for unstimulated control samples were subtracted from all test samples and percentage inhibition was determined as compared to DMSO stimulated samples. GraphPad Prism was used to determine EC50 values using a 4-parameter logistic curve fitting model. EC₅₀ ranges are reported as follows: A<0.1 μM; B 0.1-1 μM; C 1-10 μM

| | HEK-Blue | | HEK-Blue |
|---|---|---|---|
| Ex. # | IL-4 EC₅₀ | Ex. # | IL-4 EC₅₀ |
| 1 | A | 2 | B |
| 13 | C | 18 | A |
| 19 | A | 20 | A |
| 21 | B | 22 | C |
| 23 | C | 23 | C |
| 25 | C | 26 | C |
| 27 | — | 28 | C |
| 29 | C | 30 | B |
| 31 | C | 32 | C |
| 33 | C | 34 | C |
| 35 | B | 36 | C |
| 37 | C | 38 | C |
| 39 | B | 40 | A |
| 41 | B | 42 | A |
| 43 | A | 44 | A |
| 45 | A | 46 | A |
| 47 | A | 48 | B |
| 49 | B | 50 | B |
| 51 | A | 52 | A |
| 53 | A | 54 | C |
| 55 | A | | |

While this invention has been particularly shown and described with references to preferred embodiments thereof, it will be understood by those skilled in the art that various changes in form and details may be made therein without departing from the scope of the invention encompassed by the appended claims.

1. A compound represented by Formula (I) or a pharmaceutically acceptable salt thereof, 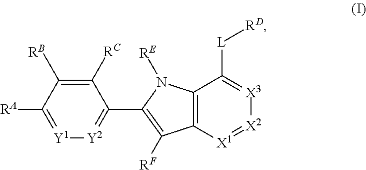
wherein;
R^{A} is selected from the group consisting of:
1) Optionally substituted 3- to 12-membered heterocycloalkyl;
2) Optionally substituted aryl;
3) Optionally substituted arylalkyl;
4) Optionally substituted heteroaryl; and
5) Optionally substituted heteroarylalkyl;
R^{B} is selected from the group consisting of hydrogen, halogen, cyano, hydroxy, optionally substituted —C₁-C₈ alkyl, optionally substituted —C₂-C₈ alkenyl, optionally substituted —C₂-C₈ alkynyl, optionally substituted —C₁-C₈ alkoxy, optionally substituted —C₃-C₈ cycloalkyl, optionally substituted 3- to 8-membered heterocycloalkyl, optionally substituted aryl, optionally substituted heteroaryl, —C(O)N(R₁)(R₂), —NR₁R₂, —SR₁, —SO₂R₁, —N(R₁)C(O)(R₂), —N(R₁)C(O)O(R₂), and —N(R₁)S(O)₂(R₂);
R^{C} is hydrogen, optionally substituted —C₁-C₈ alkyl, or R^{C1};
R^{C1} is selected from the group consisting of halogen, cyano, hydroxy, optionally substituted —C₂-C₈ alkenyl, optionally substituted —C₂-C₈ alkynyl, optionally substituted —C₁-C₈ alkoxy, optionally substituted —C₃-C₈ cycloalkyl, optionally substituted 3- to 8-membered heterocycloalkyl, optionally substituted aryl, optionally substituted heteroaryl, —C(O)N(R₁)(R₂), —NR₁R₂, —SR₁, —SO₂R₁, —N(R₁)C(O)(R₂), —N(R₁)C(O)O(R₂), and —N(R₁)S(O)₂(R₂);
R^{D} is selected from the group consisting of hydrogen, optionally substituted —C₁-C₈ alkyl, optionally substituted —C₂-C₈ alkenyl, optionally substituted —C₃-C₁₂ cycloalkyl, optionally substituted 3- to 12-membered heterocycloalkyl, optionally substituted aryl, optionally substituted arylalkyl, optionally substituted heteroaryl, and optionally substituted heteroarylalkyl;
R^{E} is selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted arylalkyl, optionally substituted heteroarylalkyl, optionally substituted aryl, and optionally substituted heteroaryl;
R^{F} is hydrogen, halogen, or R^{F1};
R^{F1} is selected from the group consisting of:
1) Cyano;
2) Optionally substituted —C₁-C₈ alkyl;
3) Optionally substituted —C₃-C₈ cycloalkyl;
4) Optionally substituted 3- to 8-membered heterocycloalkyl;
5) Optionally substituted aryl;
6) Optionally substituted heteroaryl;
7) Optionally substituted —C₁-C₈ alkoxy; and
8) —C(O)N(R₁)(R₂);
Y¹ and Y² are independently selected from N or CR₄;
X¹, X², and X³ are each independently selected from N or CR₅;
L is selected from the group consisting of —C(R₆)₂N(R₇)—, —N(R₇)C(R₆)₂—, —C(R₆)₂O—, —OC(R₆)₂—, —SO₂N(R₇)—, —N(R₇)SO₂—, —C(O)N(R₇)—, —N(R₇)C(O)—, —C(O)₂—, —OC(O)—, —N(R₇)—, —S(O)₂—, —S(O)₂N(R₇)C(O)—, and —C(O)N(R₇)S(O)₂—;
R₁ and R₂ are each independently selected from hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl, alternatively, R₁ and R₂ are taken together with the atom to which they are attached to form an optionally substituted 3-8 membered heterocyclic containing 0, 1, 2, or 3 double bonds;
R₄ is selected from the group consisting of hydrogen, halogen, cyano, hydroxy, optionally substituted —C₁-C₈ alkyl, optionally substituted —C₂-C₈ alkenyl, optionally substituted —C₂-C₈ alkynyl, optionally substituted —C₃-C₈ cycloalkyl, optionally substituted 3- to 8-membered heterocycloalkyl, optionally substituted —C₁-C₈ alkoxy, optionally substituted aryl, optionally substituted heteroaryl, —NR₁R₂, —SR₁, —SO₂R₁, —C(O)N(R₁)(R₂), —N(R₁)C(O)(R₂), —N(R₁)C(O)O(R₂), and —N(R₁)S(O)₂(R₂);
R₅ is independently selected from the group consisting of:
1) Hydrogen;
2) Halogen;
3) Cyano;
4) Hydroxy;
5) Optionally substituted alkyl;
6) Optionally substituted cycloalkyl;
7) Optionally substituted heterocycloalkyl;
8) Optionally substituted aryl;
9) Optionally substituted heteroaryl;
10) Optionally substituted —C₁-C₈ alkoxy;
11) —C(O)N(R₁)(R₂);
12) —N(R₁)C(O)(R₂);
13) —N(R₁)(R₂);
14) —SR₁;
15) —S(O)₂R₁;
16) —N(R₁)C(O)O(R₂);
17) —N(R₁)S(O)₂(R₂); and
18) —P(O)R₁R₂;
R₆ is hydrogen, halogen, optionally substituted —C₁-C₈ alkyl, optionally substituted —C₂-C₈ alkenyl, or optionally substituted —C₂-C₈ alkynyl; alternatively, two R₆ groups can take together with the carbon atom to which they are attached to form an optionally substituted —C₃-C₈ cycloalkyl or 3- to 8 membered heterocycloalkyl ring system; and
R₇ is selected from the group consisting of hydrogen, optionally substituted —C₁-C₈ alkyl, optionally substituted —C₃-C₈ cycloalkyl, optionally substituted 3- to 8 membered heterocycloalkyl, optionally substituted aryl and optionally substituted heteroaryl; and
provided that when R^{C} is hydrogen or optionally substituted —C₁-C₈ alkyl and R^{F} is hydrogen or halogen, then (1) L is not —C(O)₂—, —C(O)NH—, —CH₂O—, or CH₂NH—, and/or (2) R^{D} is not optionally substituted phenyl or optionally substituted 5- to 6-membered heteroaryl.

2. The compound of claim 1, represented by Formula (II), or a pharmaceutically acceptable salt thereof: 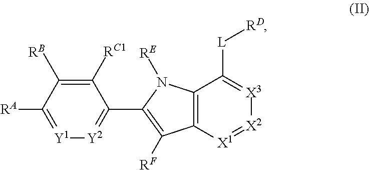
wherein R^{A}, R^{B}, R^{C1}, R^{D}, R^{E}, R^{F}, X¹, X², X³, Y¹, Y², and L are as defined in claim 1.

3. The compound of claim 1, represented by Formula (VII), 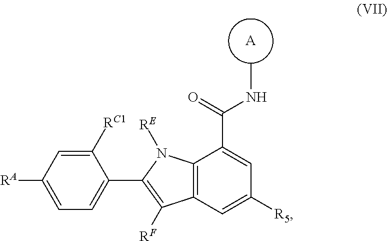
or a pharmaceutically acceptable salt thereof, wherein \textcircled{A} is selected from the group consisting of optionally substituted —C₃-C₁₂ cycloalkyl, optionally substituted 3- to 12-membered heterocycloalkyl, optionally substituted aryl and optionally substituted heteroaryl; and R^{A}, R^{C1}, R^{E}, R^{F}, and R₅ are as defined in claim 1.

4. The compound of claim 1, represented by Formula (VIII), 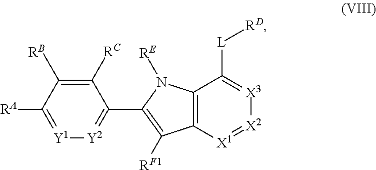
or a pharmaceutically acceptable salt thereof, wherein R^{A}, R^{B}, R^{C}, R^{D}, R^{E}, R^{F1}, X¹, X², X³, Y¹, Y², and L are as defined in claim 1.

5. The compound of claim 1, represented by Formula (XIII), 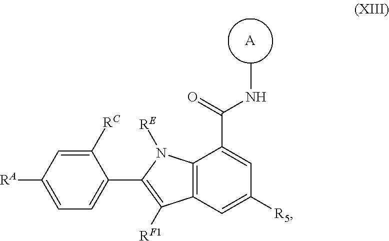
or a pharmaceutically acceptable salt thereof, wherein \textcircled{A} is selected from the group consisting of optionally substituted —C₃-C₁₂ cycloalkyl, optionally substituted 3- to 12-membered heterocycloalkyl, optionally substituted aryl and optionally substituted heteroaryl; and R^{A}, R^{C}, R^{E}, R^{F1}, and R₅ are as defined in claim 1.

6. The compound of claim 1, represented by one of Formulae (XIX-1)˜(XIX-3), 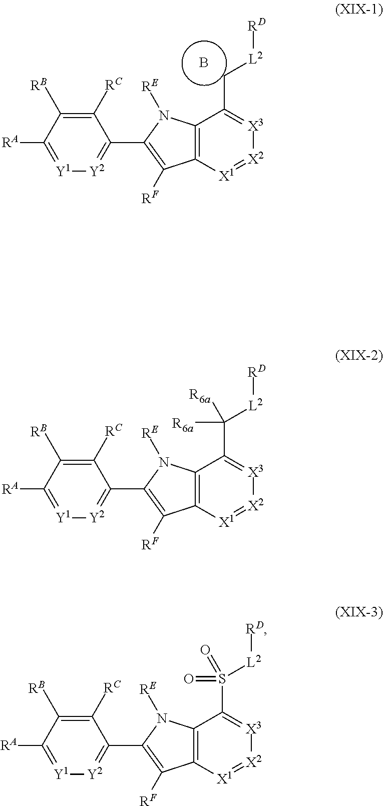
or a pharmaceutically acceptable salt thereof, wherein each R₆ₐ is independently selected from the group consisting of optionally substituted alkyl and halogen; \textcircled{B} is an optionally substituted optionally substituted —C₃-C₈ cycloalkyl or optionally substituted 3- to 8-membered heterocycloalkyl; L² is selected from the group consisting of —N(R⁷)— and —O—; and R^{A}, R^{B}, R^{C}, R^{D}, R^{E}, R^{F}, X¹, X², X³, Y¹, Y² and R⁷ are as defined in claim 1.

7. The compound of claim 1, represented by one of Formulae (XX-1)˜(XX-6), 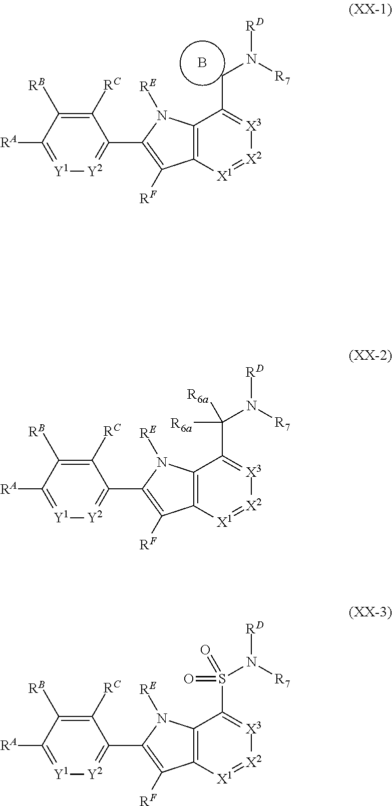 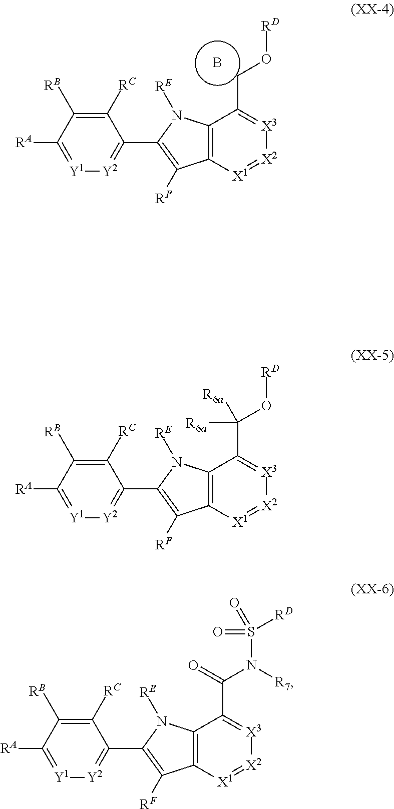
or a pharmaceutically acceptable salt thereof, wherein each R₆ₐ is independently selected from the group consisting of optionally substituted alkyl and halogen; \textcircled{B} is an optionally substituted optionally substituted —C₃-C₈ cycloalkyl or optionally substituted 3- to 8-membered heterocycloalkyl; and R^{A}, R^{B}, R^{C}, R^{D}, R^{E}, R^{F}, X¹, X², X³, Y¹, Y² and R⁷ are as defined in claim 1.

8. The compound of claim 1, represented by one of Formulae (XXV-1)˜(XXV-5), 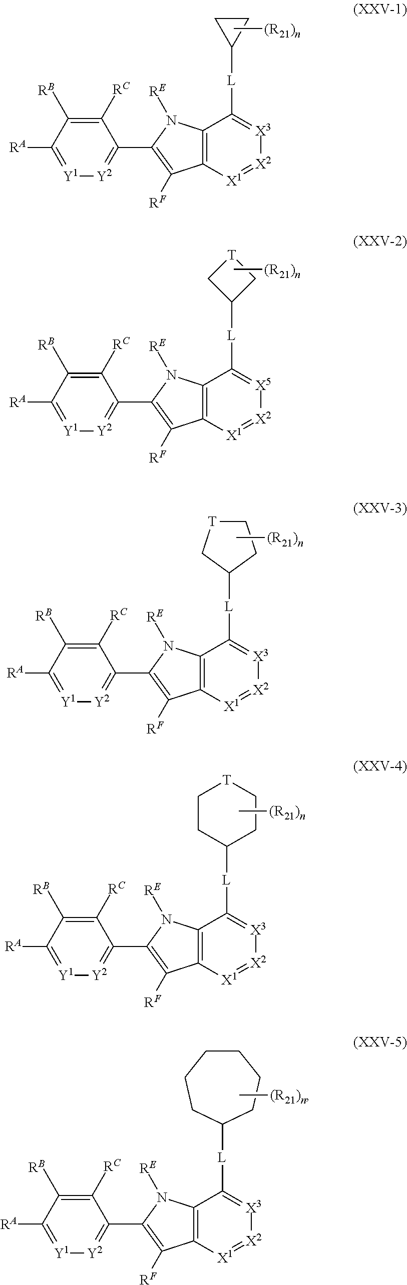
or a pharmaceutically acceptable salt thereof, wherein
R₂₁ is selected from the group consisting of:
1) Hydrogen;
2) Cyano;
3) Halogen;
4) Hydroxy;
5) Optionally substituted C₁-C₈ alkyl;
6) Optionally substituted aryl;
7) Optionally substituted heteroaryl;
8) Optionally substituted —C₁-C₈ alkoxy;
9) —C(O)N(R₁)(R₂); and
10) —CO₂H;
n is 0, 1 or 2;
alternatively, n is 2, and two geminal R₂₁ groups are taken together with the carbon atom to which they are attached to form a spiro carbocyclic or heterocyclic ring, or two adjacent R₂₁ groups are taken together with the atoms to which they are attached to form a fused carbocyclic or heterocyclic ring, or two remote R₂₁ groups are taken together with the atoms to which they are attached to form a bridge;
T is selected from the group consisting of O, S, SO₂, NR₂₂, and C(R₂₃)₂; R₂₂ is selected from the group consisting of:
1) Hydrogen;
2) Optional substituted —C₁-C₈ alkyl;
3) Optionally substituted —C₃-C₈ cycloalkyl;
4) Optionally substituted 3- to 8-membered heterocycloalkyl;
5) Optionally substituted aryl;
6) Optionally substituted heteroaryl;
7) —C(O)R₁;
8) —C(O)OR₁;
9) —C(O)N(R₁)(R₂); and
10) —S(O)₂R₁;
each R₂₃ is independently selected from the group consisting of:
1) Hydrogen;
2) Halogen;
3) Optional substituted —C₁-C₈ alkyl;
4) Optionally substituted —C₃-C₈ cycloalkyl;
5) Optionally substituted 3- to 8-membered heterocycloalkyl;
6) Optionally substituted aryl;
7) Optionally substituted heteroaryl;
8) —C(O)R₁;
9) —C(O)OR₁;
10) —C(O)N(R₁)(R₂); and
11) —S(O)₂R₁;
and R^{A}, R^{B}, R^{C}, R^{E}, R^{F}, X¹, X², X³, L, Y¹ and Y² are as defined in claim 1.

9. The compound of claim 1, represented by Formula (XXIX), 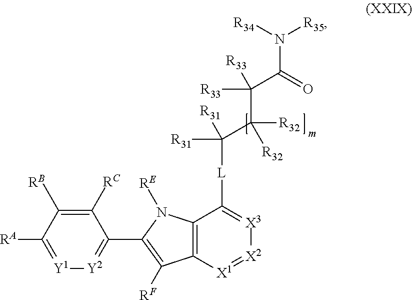
or a pharmaceutically acceptable salt thereof, wherein
each R₃₁ is independently selected from the group consisting of hydrogen, halogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted heteroaryl, and —C(O)N(R₁)(R₂);
each R₃₂ is independently selected from the group consisting of hydrogen, halogen, and optionally substituted —C₁-C₈ alkyl;
alternatively, two R₃₂ groups are taken together with the carbon atom to which they are attached to form an optionally substituted —C₃-C₈ cycloalkyl or optionally substituted 3- to 8-membered heterocycloalkyl;
each R₃₃ is independently selected from the group consisting of hydrogen, halogen, and —C₁-C₈ optionally substituted alkyl;
R₃₄ is R₁; R₃₅ is R₂;
m is 0, 1, or 2; and
R₁, R₂, R^{A}, R^{B}, R^{C}, R^{E}, R^{F}, L, X¹, X², X³, Y¹, and Y² are as defined in claim 1.

10. The compound of claim 1, represented by Formula (XXXVIII), 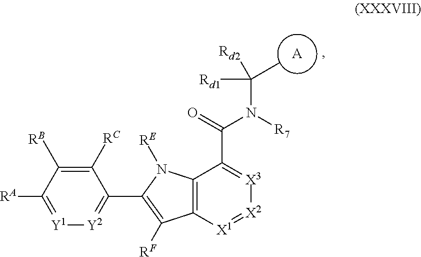
or a pharmaceutically acceptable salt thereof, wherein \textcircled{A} is selected from the group consisting of optionally substituted —C₃-C₁₂ cycloalkyl, optionally substituted 3- to 12-membered heterocycloalkyl, optionally substituted aryl and optionally substituted heteroaryl; R_{d1} and R_{d2} are each independently selected from hydrogen, halogen, and optionally substituted —C₁-C₆ alkyl;
and R^{A}, R^{B}, R^{C}, R^{E}, R^{F}, X¹, X², X³, Y¹, Y², and R⁷ are as defined in claim 1.

11. The compound of claim 1, represented by Formula (XXXIX), 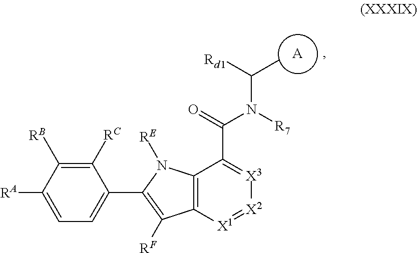
or a pharmaceutically acceptable salt thereof, wherein \textcircled{A} is selected from the group consisting of optionally substituted —C₃-C₁₂ cycloalkyl, optionally substituted 3- to 12-membered heterocycloalkyl, optionally substituted aryl and optionally substituted heteroaryl; R_{d1} is selected from hydrogen, halogen, and optionally substituted —C₁-C₆ alkyl; and R^{A}, R^{B}, R^{C}, R^{E}, R^{F}, X¹, X², X³, and R⁷ are as defined in claim 1.

12. A Compound selected from the compounds set forth below, or a pharmaceutically acceptable salt thereof:

| Com- | |
|---|---|
| pound | Structure |
| 1 | 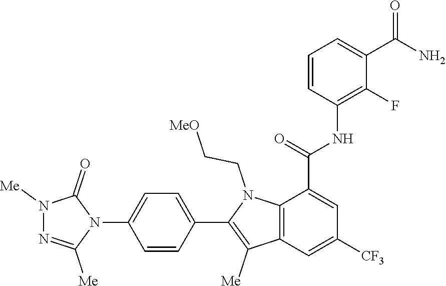 [SMILES: COCCn1c(-c2ccc(-n3c(C)nn(C)c3=O)cc2)c(C)c2cc(C(F)(F)F)cc(C(=O)Nc3cccc(C(N)=O)c3F)c21] |
| 2 | 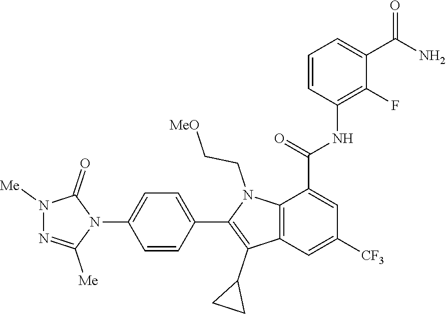 [SMILES: COCCn1c(-c2ccc(-n3c(C)nn(C)c3=O)cc2)c(C2CC2)c2cc(C(F)(F)F)cc(C(=O)Nc3cccc(C(N)=O)c3F)c21] |
| 3 | 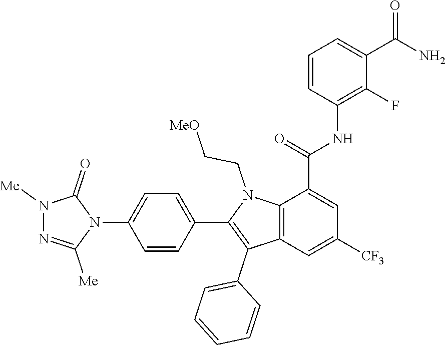 [SMILES: COCCn1c(-c2ccc(-n3c(C)nn(C)c3=O)cc2)c(-c2ccccc2)c2cc(C(F)(F)F)cc(C(=O)Nc3cccc(C(N)=O)c3F)c21] |
| 4 | 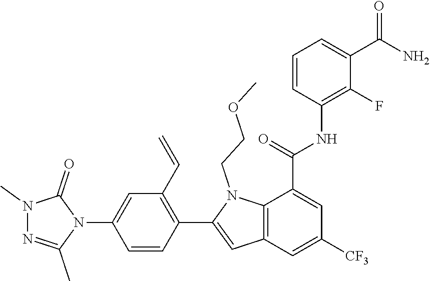 [SMILES: C=Cc1cc(-n2c(C)nn(C)c2=O)ccc1-c1cc2cc(C(F)(F)F)cc(C(=O)Nc3cccc(C(N)=O)c3F)c2n1CCOC] |
| 5 | 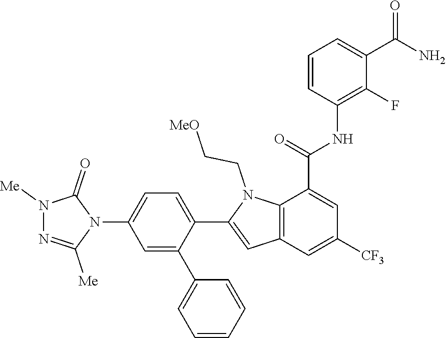 [SMILES: COCCn1c(-c2ccc(-n3c(C)nn(C)c3=O)cc2-c2ccccc2)cc2cc(C(F)(F)F)cc(C(=O)Nc3cccc(C(N)=O)c3F)c21] |
| 6 | 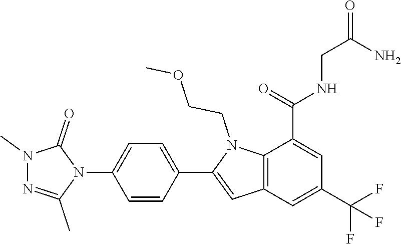 [SMILES: COCCn1c(-c2ccc(-n3c(C)nn(C)c3=O)cc2)cc2cc(C(F)(F)F)cc(C(=O)NCC(N)=O)c21] |
| 7 | 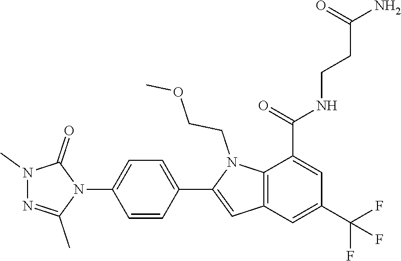 [SMILES: COCCn1c(-c2ccc(-n3c(C)nn(C)c3=O)cc2)cc2cc(C(F)(F)F)cc(C(=O)NCCC(N)=O)c21] |
| 8 | 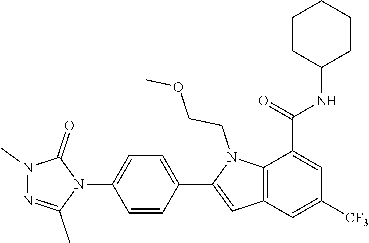 [SMILES: COCCn1c(-c2ccc(-n3c(C)nn(C)c3=O)cc2)cc2cc(C(F)(F)F)cc(C(=O)NC3CCCCC3)c21] |
| 9 | 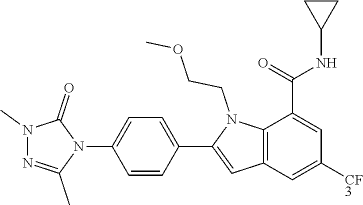 [SMILES: COCCn1c(-c2ccc(-n3c(C)nn(C)c3=O)cc2)cc2cc(C(F)(F)F)cc(C(=O)NC3CC3)c21] |
| 10 | 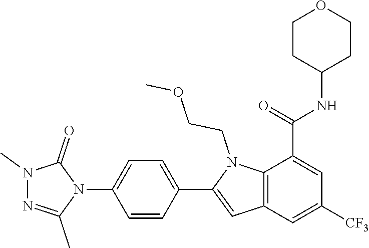 [SMILES: COCCn1c(-c2ccc(-n3c(C)nn(C)c3=O)cc2)cc2cc(C(F)(F)F)cc(C(=O)NC3CCOCC3)c21] |
| 11 | 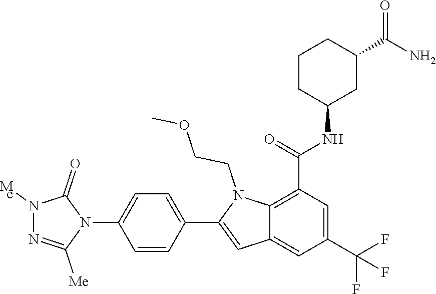 [SMILES: COCCn1c(-c2ccc(-n3c(C)nn(C)c3=O)cc2)cc2cc(C(F)(F)F)cc(C(=O)N[C@H]3CCC[C@H](C(N)=O)C3)c21] |
| 12 | 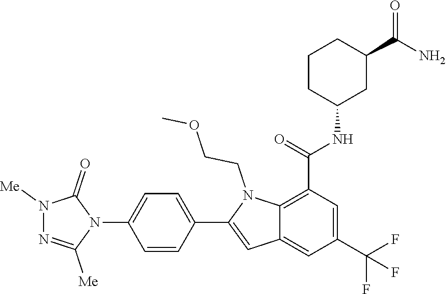 [SMILES: COCCn1c(-c2ccc(-n3c(C)nn(C)c3=O)cc2)cc2cc(C(F)(F)F)cc(C(=O)N[C@@H]3CCC[C@@H](C(N)=O)C3)c21] |
| 13 | 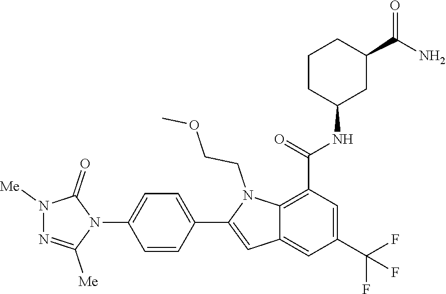 [SMILES: COCCn1c(-c2ccc(-n3c(C)nn(C)c3=O)cc2)cc2cc(C(F)(F)F)cc(C(=O)N[C@H]3CCC[C@@H](C(N)=O)C3)c21] |
| 14 | 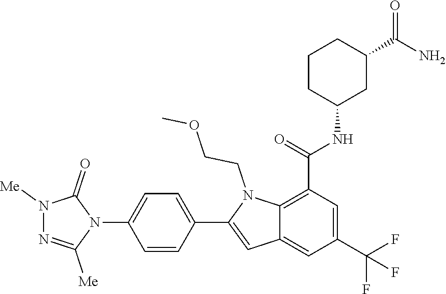 [SMILES: COCCn1c(-c2ccc(-n3c(C)nn(C)c3=O)cc2)cc2cc(C(F)(F)F)cc(C(=O)N[C@@H]3CCC[C@H](C(N)=O)C3)c21] |
| 15 | 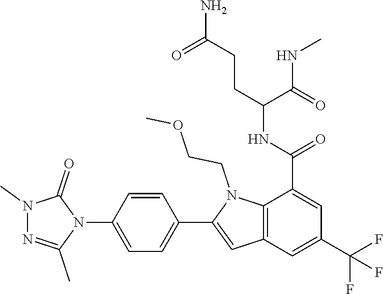 [SMILES: CNC(=O)C(CCC(N)=O)NC(=O)c1cc(C(F)(F)F)cc2cc(-c3ccc(-n4c(C)nn(C)c4=O)cc3)n(CCOC)c12] |
| 16 | 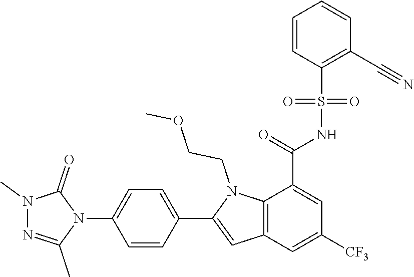 [SMILES: COCCn1c(-c2ccc(-n3c(C)nn(C)c3=O)cc2)cc2cc(C(F)(F)F)cc(C(=O)NS(=O)(=O)c3ccccc3C#N)c21] |
| 17 | 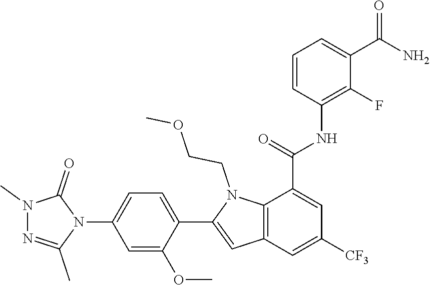 [SMILES: COCCn1c(-c2ccc(-n3c(C)nn(C)c3=O)cc2OC)cc2cc(C(F)(F)F)cc(C(=O)Nc3cccc(C(N)=O)c3F)c21] |
| 18 | 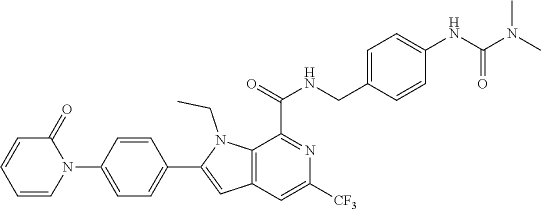 [SMILES: CCn1c(-c2ccc(-n3ccccc3=O)cc2)cc2cc(C(F)(F)F)nc(C(=O)NCc3ccc(NC(=O)N(C)C)cc3)c21] |
| 19 | 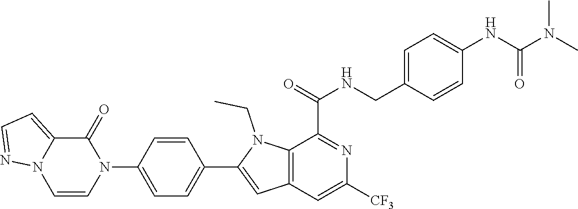 [SMILES: CCn1c(-c2ccc(-n3ccn4nccc4c3=O)cc2)cc2cc(C(F)(F)F)nc(C(=O)NCc3ccc(NC(=O)N(C)C)cc3)c21] |
| 20 | 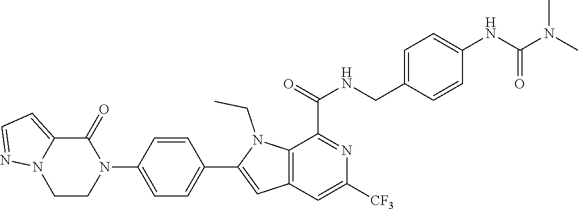 [SMILES: CCn1c(-c2ccc(N3CCn4nccc4C3=O)cc2)cc2cc(C(F)(F)F)nc(C(=O)NCc3ccc(NC(=O)N(C)C)cc3)c21] |
| 21 | 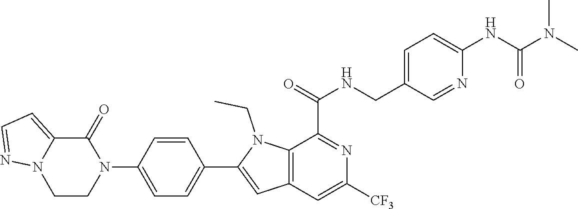 [SMILES: CCn1c(-c2ccc(N3CCn4nccc4C3=O)cc2)cc2cc(C(F)(F)F)nc(C(=O)NCc3ccc(NC(=O)N(C)C)nc3)c21] |
| 22 | 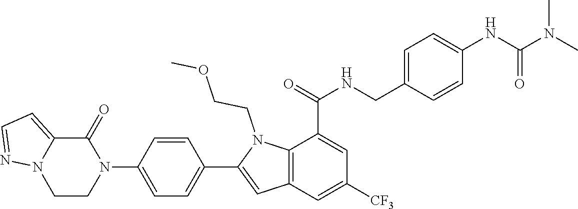 [SMILES: COCCn1c(-c2ccc(N3CCn4nccc4C3=O)cc2)cc2cc(C(F)(F)F)cc(C(=O)NCc3ccc(NC(=O)N(C)C)cc3)c21] |
| 23 | 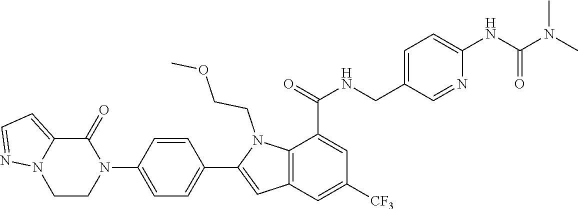 [SMILES: COCCn1c(-c2ccc(N3CCn4nccc4C3=O)cc2)cc2cc(C(F)(F)F)cc(C(=O)NCc3ccc(NC(=O)N(C)C)nc3)c21] |
| 24 | 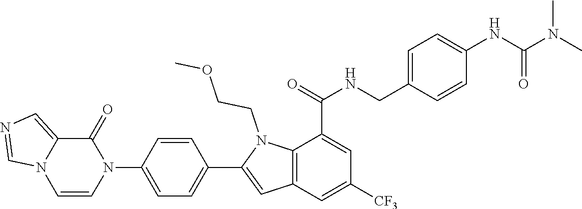 [SMILES: COCCn1c(-c2ccc(-n3ccn4cncc4c3=O)cc2)cc2cc(C(F)(F)F)cc(C(=O)NCc3ccc(NC(=O)N(C)C)cc3)c21] |
| 25 | 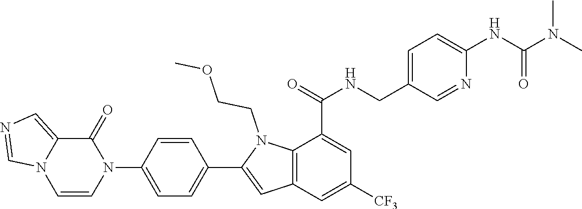 [SMILES: COCCn1c(-c2ccc(-n3ccn4cncc4c3=O)cc2)cc2cc(C(F)(F)F)cc(C(=O)NCc3ccc(NC(=O)N(C)C)nc3)c21] |
| 26 | 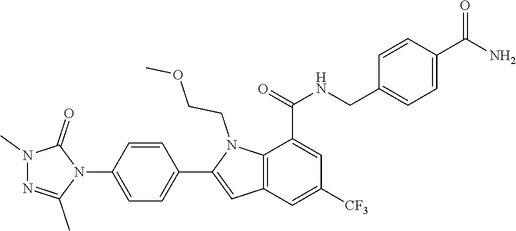 [SMILES: COCCn1c(-c2ccc(-n3c(C)nn(C)c3=O)cc2)cc2cc(C(F)(F)F)cc(C(=O)NCc3ccc(C(N)=O)cc3)c21] |
| 27 | 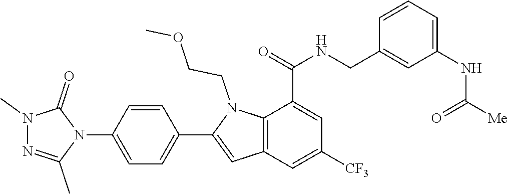 [SMILES: COCCn1c(-c2ccc(-n3c(C)nn(C)c3=O)cc2)cc2cc(C(F)(F)F)cc(C(=O)NCc3cccc(NC(C)=O)c3)c21] |
| 28 | 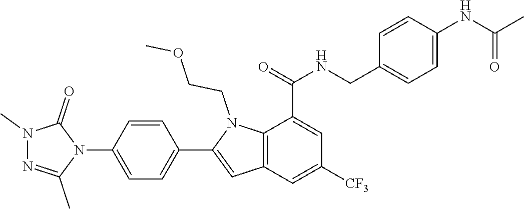 [SMILES: COCCn1c(-c2ccc(-n3c(C)nn(C)c3=O)cc2)cc2cc(C(F)(F)F)cc(C(=O)NCc3ccc(NC(C)=O)cc3)c21] |
| 29 | 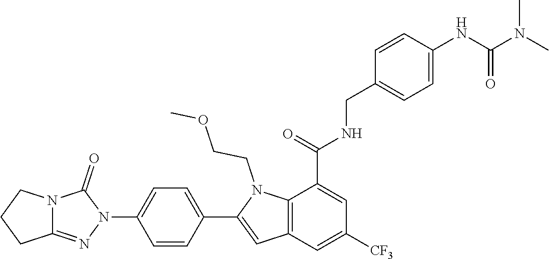 [SMILES: COCCn1c(-c2ccc(-n3nc4n(c3=O)CCC4)cc2)cc2cc(C(F)(F)F)cc(C(=O)NCc3ccc(NC(=O)N(C)C)cc3)c21] |
| 30 | 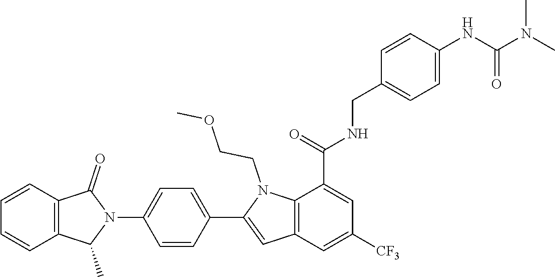 [SMILES: COCCn1c(-c2ccc(N3C(=O)c4ccccc4[C@H]3C)cc2)cc2cc(C(F)(F)F)cc(C(=O)NCc3ccc(NC(=O)N(C)C)cc3)c21] |
| 31 | 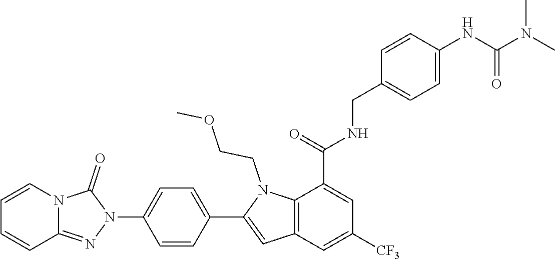 [SMILES: COCCn1c(-c2ccc(-n3nc4ccccn4c3=O)cc2)cc2cc(C(F)(F)F)cc(C(=O)NCc3ccc(NC(=O)N(C)C)cc3)c21] |
| 32 | 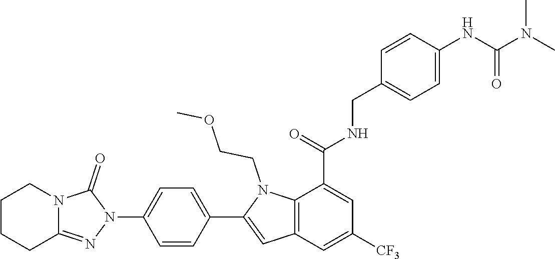 [SMILES: COCCn1c(-c2ccc(-n3nc4n(c3=O)CCCC4)cc2)cc2cc(C(F)(F)F)cc(C(=O)NCc3ccc(NC(=O)N(C)C)cc3)c21] |
| 33 | 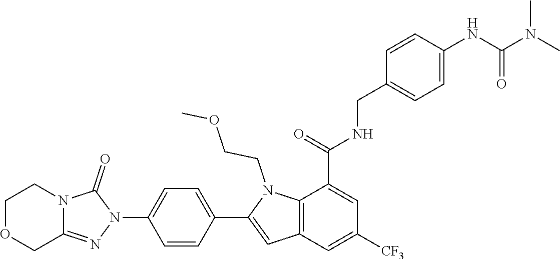 [SMILES: COCCn1c(-c2ccc(-n3nc4n(c3=O)CCOC4)cc2)cc2cc(C(F)(F)F)cc(C(=O)NCc3ccc(NC(=O)N(C)C)cc3)c21] |
| 34 | 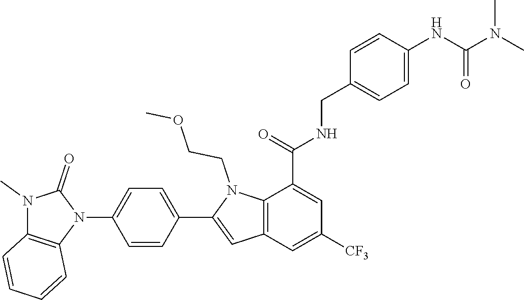 |
| 35 | 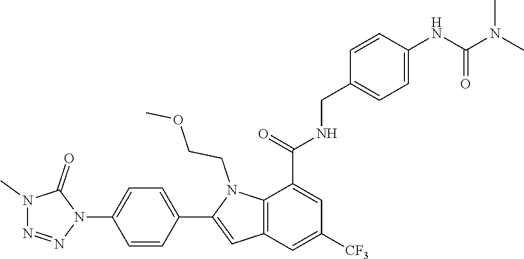 |
| 36 | 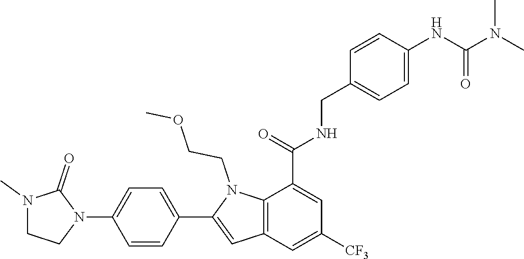 |
| 37 | 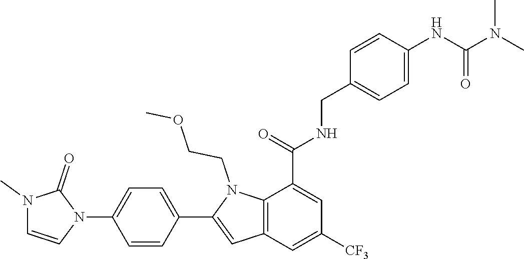 |
| 38 | 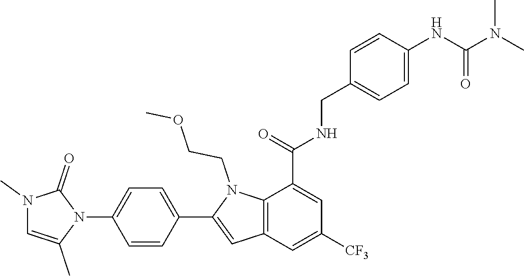 |
| 39 | 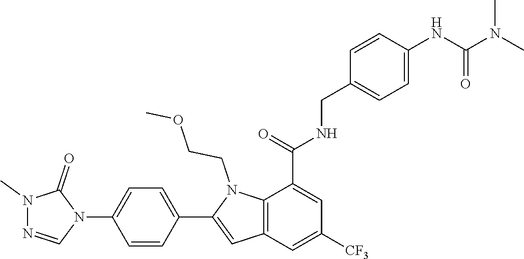 |
| 40 | 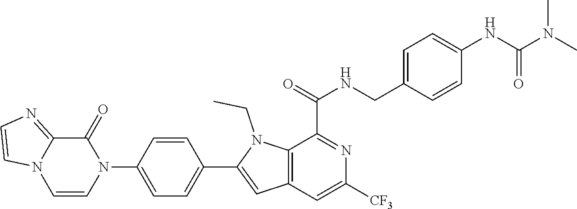 [SMILES: CCn1c(-c2ccc(-n3ccn4ccnc4c3=O)cc2)cc2cc(C(F)(F)F)nc(C(=O)NCc3ccc(NC(=O)N(C)C)cc3)c21] |
| 41 | 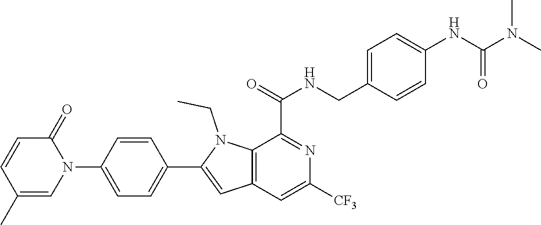 [SMILES: CCn1c(-c2ccc(-n3cc(C)ccc3=O)cc2)cc2cc(C(F)(F)F)nc(C(=O)NCc3ccc(NC(=O)N(C)C)cc3)c21] |
| 42 | 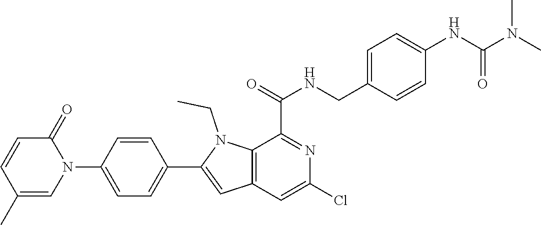 [SMILES: CCn1c(-c2ccc(-n3cc(C)ccc3=O)cc2)cc2cc(Cl)nc(C(=O)NCc3ccc(NC(=O)N(C)C)cc3)c21] |
| 43 | 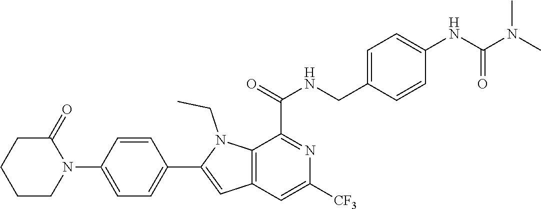 [SMILES: CCn1c(-c2ccc(N3CCCCC3=O)cc2)cc2cc(C(F)(F)F)nc(C(=O)NCc3ccc(NC(=O)N(C)C)cc3)c21] |
| 44 | 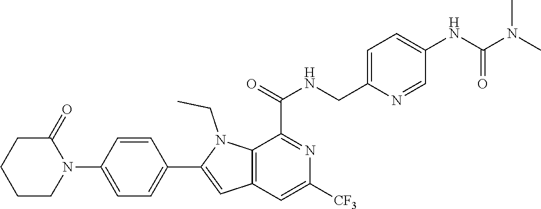 [SMILES: CCn1c(-c2ccc(N3CCCCC3=O)cc2)cc2cc(C(F)(F)F)nc(C(=O)NCc3ccc(NC(=O)N(C)C)cn3)c21] |
| 45 | 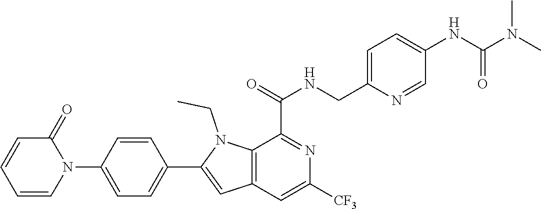 [SMILES: CCn1c(-c2ccc(-n3ccccc3=O)cc2)cc2cc(C(F)(F)F)nc(C(=O)NCc3ccc(NC(=O)N(C)C)cn3)c21] |
| 46 | 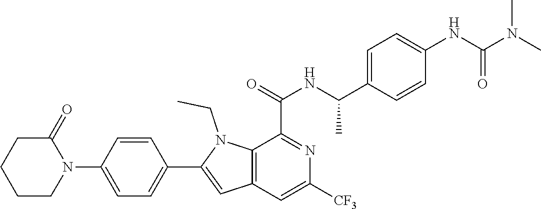 [SMILES: CCn1c(-c2ccc(N3CCCCC3=O)cc2)cc2cc(C(F)(F)F)nc(C(=O)N[C@@H](C)c3ccc(NC(=O)N(C)C)cc3)c21] |
| 47 | 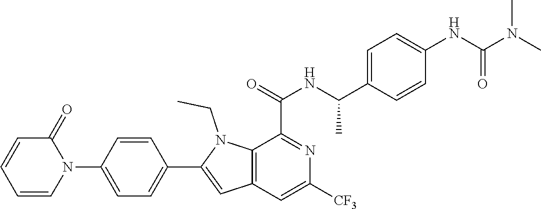 [SMILES: CCn1c(-c2ccc(-n3ccccc3=O)cc2)cc2cc(C(F)(F)F)nc(C(=O)N[C@@H](C)c3ccc(NC(=O)N(C)C)cc3)c21] |
| 48 | 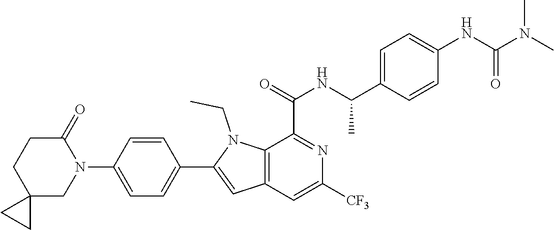 [SMILES: CCn1c(-c2ccc(N3CC4(CCC3=O)CC4)cc2)cc2cc(C(F)(F)F)nc(C(=O)N[C@@H](C)c3ccc(NC(=O)N(C)C)cc3)c21] |
| 49 | 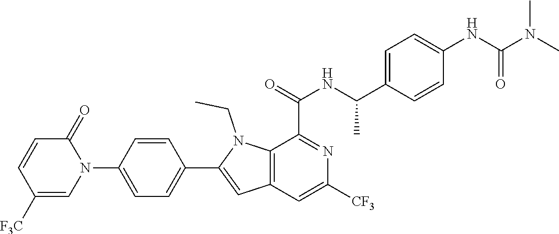 [SMILES: CCn1c(-c2ccc(-n3cc(C(F)(F)F)ccc3=O)cc2)cc2cc(C(F)(F)F)nc(C(=O)N[C@@H](C)c3ccc(NC(=O)N(C)C)cc3)c21] |
| 50 | 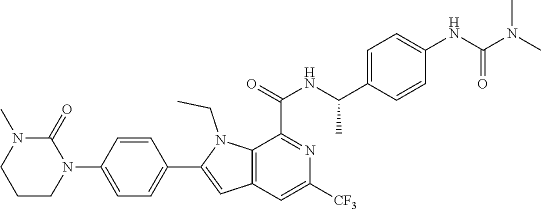 [SMILES: CCn1c(-c2ccc(N3CCCN(C)C3=O)cc2)cc2cc(C(F)(F)F)nc(C(=O)N[C@@H](C)c3ccc(NC(=O)N(C)C)cc3)c21] |
| 51 | 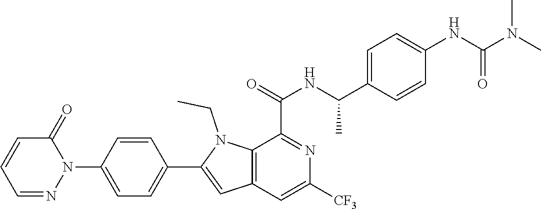 [SMILES: CCn1c(-c2ccc(-n3ncccc3=O)cc2)cc2cc(C(F)(F)F)nc(C(=O)N[C@@H](C)c3ccc(NC(=O)N(C)C)cc3)c21] |
| 52 | 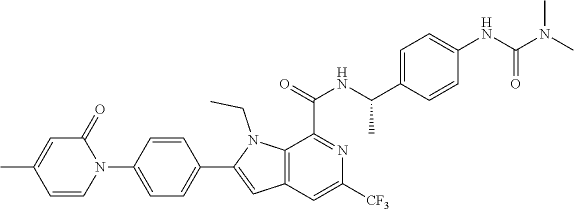 |
| 53 | 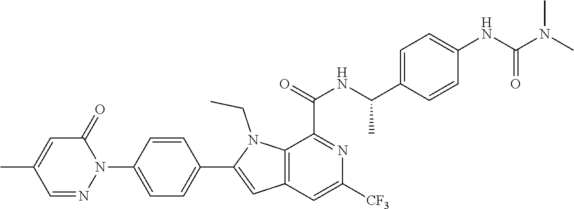 |
| 54 | 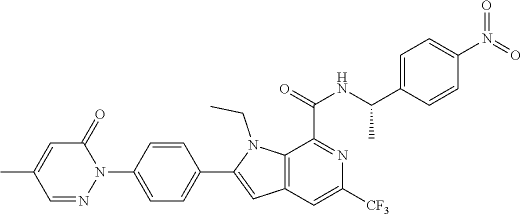 [SMILES: CCn1c(-c2ccc(-n3ncc(C)cc3=O)cc2)cc2cc(C(F)(F)F)nc(C(=O)N[C@@H](C)c3ccc([N+](=O)[O-])cc3)c21] |
| 55 | 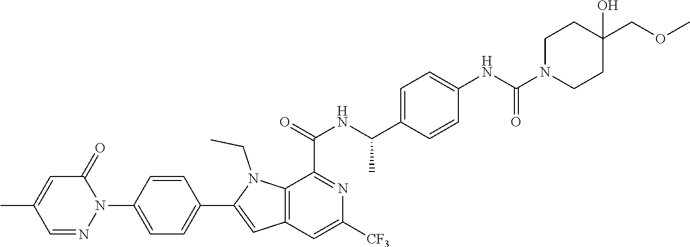 |
| 1a | 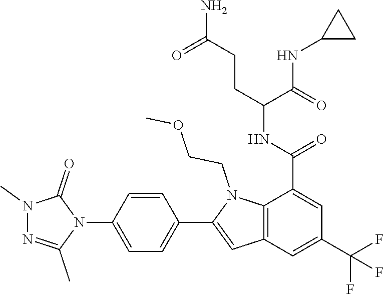 [SMILES: COCCn1c(-c2ccc(-n3c(C)nn(C)c3=O)cc2)cc2cc(C(F)(F)F)cc(C(=O)NC(CCC(N)=O)C(=O)NC3CC3)c21] |
| 2a | 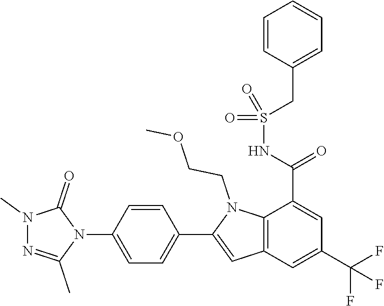 [SMILES: COCCn1c(-c2ccc(-n3c(C)nn(C)c3=O)cc2)cc2cc(C(F)(F)F)cc(C(=O)NS(=O)(=O)Cc3ccccc3)c21] |
| 3a | 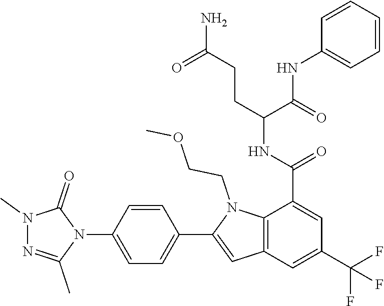 [SMILES: COCCn1c(-c2ccc(-n3c(C)nn(C)c3=O)cc2)cc2cc(C(F)(F)F)cc(C(=O)NC(CCC(N)=O)C(=O)Nc3ccccc3)c21] |
| 4a | 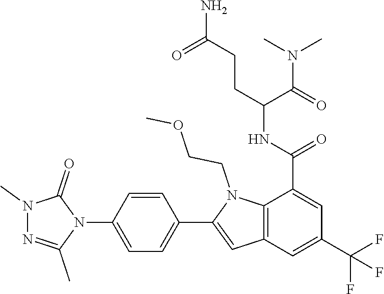 [SMILES: COCCn1c(-c2ccc(-n3c(C)nn(C)c3=O)cc2)cc2cc(C(F)(F)F)cc(C(=O)NC(CCC(N)=O)C(=O)N(C)C)c21] |
| 5a | 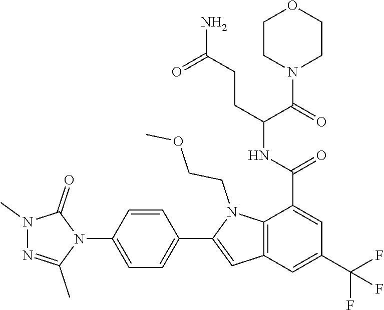 [SMILES: COCCn1c(-c2ccc(-n3c(C)nn(C)c3=O)cc2)cc2cc(C(F)(F)F)cc(C(=O)NC(CCC(N)=O)C(=O)N3CCOCC3)c21] |
| 6a | 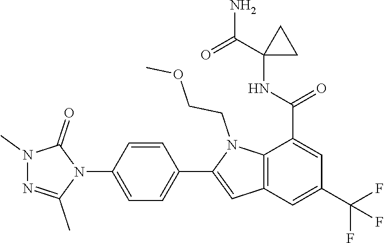 [SMILES: COCCn1c(-c2ccc(-n3c(C)nn(C)c3=O)cc2)cc2cc(C(F)(F)F)cc(C(=O)NC3(C(N)=O)CC3)c21] |
| 7a | 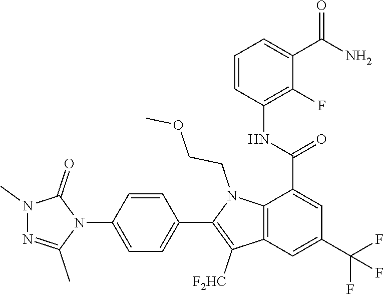 [SMILES: COCCn1c(-c2ccc(-n3c(C)nn(C)c3=O)cc2)c(C(F)F)c2cc(C(F)(F)F)cc(C(=O)Nc3cccc(C(N)=O)c3F)c21] |
| 8a | 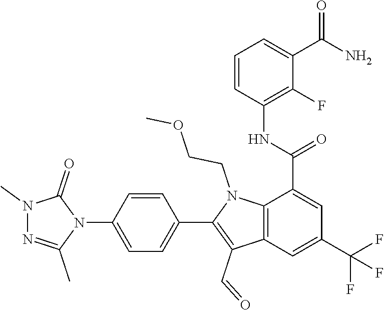 [SMILES: COCCn1c(-c2ccc(-n3c(C)nn(C)c3=O)cc2)c(C=O)c2cc(C(F)(F)F)cc(C(=O)Nc3cccc(C(N)=O)c3F)c21] |
| 9a | 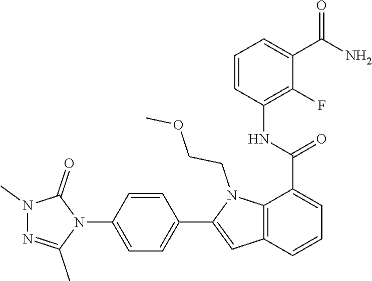 [SMILES: COCCn1c(-c2ccc(-n3c(C)nn(C)c3=O)cc2)cc2cccc(C(=O)Nc3cccc(C(N)=O)c3F)c21] |
| 10a | 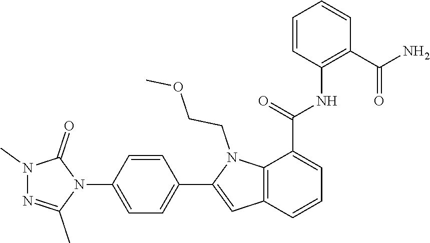 [SMILES: COCCn1c(-c2ccc(-n3c(C)nn(C)c3=O)cc2)cc2cccc(C(=O)Nc3ccccc3C(N)=O)c21] |
| 11a | 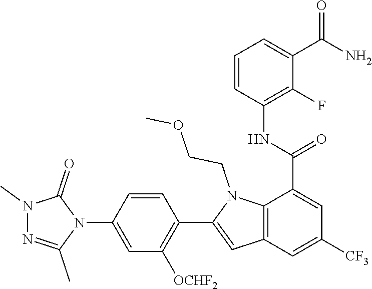 [SMILES: COCCn1c(-c2ccc(-n3c(C)nn(C)c3=O)cc2OC(F)F)cc2cc(C(F)(F)F)cc(C(=O)Nc3cccc(C(N)=O)c3F)c21] |
| 12a | 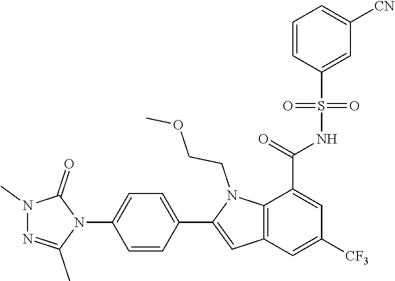 [SMILES: COCCn1c(-c2ccc(-n3c(C)nn(C)c3=O)cc2)cc2cc(C(F)(F)F)cc(C(=O)NS(=O)(=O)c3cccc(C#N)c3)c21] |
| 13a | 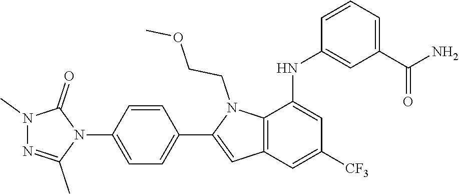 [SMILES: COCCn1c(-c2ccc(-n3c(C)nn(C)c3=O)cc2)cc2cc(C(F)(F)F)cc(Nc3cccc(C(N)=O)c3)c21] |
| 14a | 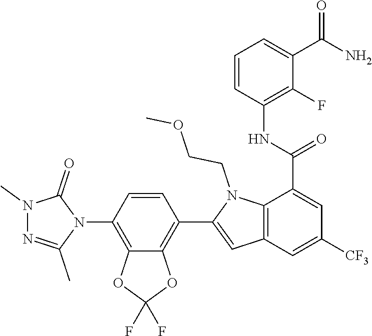 [SMILES: COCCn1c(-c2ccc(-n3c(C)nn(C)c3=O)c3c2OC(F)(F)O3)cc2cc(C(F)(F)F)cc(C(=O)Nc3cccc(C(N)=O)c3F)c21] |

13. A pharmaceutical composition comprising the compound of claim 1 and a pharmaceutically acceptable carrier.

14. A method for treating a disease, disorder, or condition where modulation of STAT6 or STAT3 is implicated, wherein the method comprises administering to a system or subject in need of such treatment an effective amount of a compound of claim 1.

15. The method of claim 14, wherein said disease, disorder or condition is an allergic disease or an inflammatory disease.

16. The method of claim 15, wherein said the disease, disorder, or condition is selected from the group consisting of chronic obstructive pulmonary disease, atopic dermatitis, bronchial asthma, bullous pemphigoid, nasal polyps, chronic sinusitis, allergic rhinitis, eosinophilic esophagitis, prurigo, and urticaria.

17. The method of claim 14, wherein said disease, disorder, or condition is selected from the group consisting of psoriasis, psoriatic arthritis, rheumatoid arthritis, and inflammatory bowel disease.
